(19) United States
(12) Patent Application Publication
(10) Pub. No.: US 2025/0255686 A1
(43) Pub. Date: Aug. 14, 2025

(54) SLAVE-SIDE EXECUTION APPARATUS AND MEDICAL SYSTEM

(71) Applicant: ZHICHENG MEDICAL TECHNOLOGY (JIAXING) CO., LTD., Haining City (CN)

(72) Inventors: Dongjing LI, Suzhou (CN); Sixin WANG, Suzhou (CN); Yi ZHANG, Suzhou (CN)

(21) Appl. No.: 19/191,375

(22) Filed: Apr. 28, 2025

Related U.S. Application Data
(63) Continuation-in-part of application No. PCT/CN2023/126853, filed on Oct. 26, 2023.

(30) Foreign Application Priority Data
Oct. 26, 2022 (CN) 2022113269627
Sep. 26, 2023 (CN) 2023112444055
Oct. 23, 2023 (CN) 2023113771158
Oct. 26, 2023 (WO) PCT/CN2023/126853
Sep. 29, 2024 (CN) 2024113791419

(51) Int. Cl. A61B 34/37; A61M 25/01; A61M 25/02

(52) U.S. Cl. CPC A61B 34/37; A61M 25/0113; A61M 25/02; A61M 2025/0293

(57) ABSTRACT

A slave-side execution apparatus and a medical system are provided. The slave-side execution apparatus is used to convey a guidewire and/or a catheter, and includes a main body power device, a movable power device, a first carrying device and a first manipulation device. The main body power device includes a main body driving device. The movable power device includes a first driving device and is used to move forward and backward along a conveying direction under the driving of the main body driving device. The first carrying device is moved synchronously with the movable power device and is used to carry a medical apparatus having a catheter. The first manipulation device is disposed on the first carrying device, and is used to control the guidewire to move forward and backward along the conveying direction under the driving of the first driving device.

### CROSS REFERENCE

The present application is a bypass continuation-in-part application claiming priority from International Application No. PCT/CN/2023/126853, filed on Oct. 26, 2023, pending, entitled “Slave-side Execution Apparatus and Medical System”, and a Chinese Patent Application No. CN202411379141.9 filed on Sep. 30, 2024, pending, entitled “Conveying Device, Conveying Apparatus and Surgical Robotic System”, the entire contents of which are incorporated herein by reference.

### BACKGROUND

The present application generally relates to the technical field of surgical robots, and more particularly to a slave-side execution apparatus for conveying a guidewire and/or a catheter and a medical system having the same.

With the continuous development of science and technology, various types of minimally invasive cardiovascular implant intervention surgeries have emerged.

During the surgery, DSA (Digital Subtraction Angiography) will emit X-rays, and the lead protective clothing worn by doctors cannot completely protect the doctors. Doctors are prone to cancer and other diseases if they are exposed to radiation for a long time. Another situation is that the lead protective clothing is relatively heavy, and the doctors wearing it for a long time will also cause a certain load on the body, affecting the surgical operation. In addition, some implant intervention surgeries (such as aortic valve replacement) are complex in operation, and require the cooperation of several doctors, and sometimes require doctors to operate by experience and feeling, which makes the surgery difficult and risky.

### SUMMARY

A series of simplified concepts are introduced into the portion of Summary, which would be further illustrated in the portion of the Detailed Description. The Summary of the present application does not mean attempting to define the key features and essential technical features of the technical solution claimed to be protected, let alone determining the protection scope of the technical solution claimed to be protected.

In order to at least partially solve the above problems, a first aspect of the present application provides a slave-side execution apparatus for conveying a guidewire and/or a catheter, the slave-side execution apparatus comprising:

a main body power device used to be installed beside a surgical bed and being immovable relative to the surgical bed, the main body power device comprising a main body driving device for providing a driving force;

a movable power device connected to the main power device and used to move forward and backward relative to the main power device along a conveying direction of the guidewire and/or the catheter under the driving of the main body driving device, the movable power device comprising a first driving device for providing driving force;

a first carrying device detachably connected to the movable power device and being movable synchronously with the movable power device along the conveying direction relative to the main body power device, the first carrying device being used to carry a medical apparatus comprising the catheter; and

a first manipulation device disposed on the first carrying device and used to control the guidewire to move forward and backward relative to the first carrying device along the conveying direction under the driving of the first driving device.

According to the present application, the slave-side execution apparatus of the slave-side may be controlled to operate by the doctors in the main control room, thereby preventing the doctors form being exposed to radiation of the ray. The components of the slave-side execution apparatus for contacting the guidewire and/or the catheter are passive components (without the need to connect to a power source), may be configured as consumables, are easy to install, and infection may be avoided.

Optionally, the movable power device comprises a second driving device for providing a driving force; and

the first carrying device comprises a second manipulation device used to contact the medical apparatus, and the second manipulation device being connected to the second driving device to manipulate the medical apparatus to perform a medical function under the driving of the second driving device.

According to the present application, the components of the slave-side execution apparatus for contacting the guidewire and/or the catheter are passive components (without the need to connect to a power source), may be configured as consumables, are easy to install, and infection may be avoided.

Optionally, the slave-side execution apparatus further comprises:

a movable assembly disposed on the main body power device and configured to be movable forward and backward relative to the main body power device along the conveying direction under the driving of the main body driving device, wherein the movable power device is connected to the movable assembly;

a first transmission device connected between the first driving device and the first manipulation device for transferring the driving force of the first driving device to the first manipulation device;

a second transmission device connected between the second driving device and the second manipulation device for transferring the driving force of the second driving device to the second manipulation device.

According to the present application, the passive components of the slave-side execution apparatus are in transmission connection to the driving device via a transmission structure.

Optionally, the first manipulation device comprises at least one pair of rollers which are arranged oppositely, the axes of the rollers are parallel to each other and the rollers are configured to be rotatable about their own axes, and the pair of the rollers are used to jointly clamp the guidewire;

the first transmission device is connected between the first driving device and at least one of the pair of the rollers to rotate the at least one of the pair of the rollers.

According to the present application, the guidewire is clamped by a pair of rollers and is conveyed by the rolling of the rollers.

Optionally, the first transmission device comprises:

a first power shaft disposed on the movable power device and configured to be rotatable about its own axis relative to the movable power device;

a first transmission assembly disposed on the movable power device and connected between the first driving device and the first power shaft such that the first power shaft is rotatable under the driving of the first driving device; and

a first power transfer device disposed on the first carrying device and connected between the first manipulation device and the first power shaft such that the first manipulation device is driven to operate by the first power shaft drives.

According to the present application, the first transmission device comprises the first power shaft, which is simple to control.

Optionally, the first manipulation device comprises:

a first manipulation device base connected to the first carrying device;

at least one first roller disposed on the first manipulation device base, an axis of the first roller being not parallel to the conveying direction, the first roller being configured to be rotatable about its axis relative to the first manipulation device base; and

at least one second roller disposed on the first manipulation device base and arranged opposite to the first roller so as to clamp the guidewire along with the first roller, the second roller being configured to be rotatable about its axis relative to the first manipulation device base;

wherein the first roller and the second roller are both used to be connected to the first transmission device so as to rotate under the driving of the first transmission device, and the slave-side execution apparatus is configured such that an rotation direction of the first roller is opposite to an rotation direction of the second roller.

According to the present application, the first roller and the second roller are both configured as driving rollers, so that the slave-side execution apparatus has sufficient driving force to convey the guidewire. Moreover, the first roller and the second roller are driven by the same driving device, which may ensure that they are rotated synchronously.

Optionally, the first manipulation device further comprises:

a second fixed seat disposed on the first manipulation device base, wherein the second roller is disposed on the second fixed seat; and

a first movable seat disposed on the first manipulation device base and being movable between a first position and a second position relative to the second fixed seat along a first direction, wherein the first roller is disposed on the first movable seat;

wherein the first direction is perpendicular to a plane determined by the conveying direction and the axis of the first roller, and a distance between the first movable seat and the second fixed seat when the first movable seat is in the first position is less than a distance between the first movable seat and the second fixed seat when the first movable seat is in the second position.

According to the present application, the distance between the first roller and the second roller is adjustable. When the distance between them is far, it is convenient to take and place the guidewire, and when the distance between them is close, the guidewire may be clamped.

Optionally, the first manipulation device further comprises:

a first biasing assembly connected between the second fixed seat and the first movable seat for providing a force to move the first movable seat away from the second fixed seat along the first direction; and

a first pressing device disposed on the first manipulation device base and connected to the first movable seat for operation by a user to bring the first movable seat close to the second fixed seat along the first direction.

According to the present application, the distance between the first roller and the second roller is adjusted by the first biasing assembly and the first pressing device.

Optionally, the first pressing device comprises:

a cam connected to the first manipulation device base and being rotatable relative to the first manipulation device base about a rotation axis extending along a second direction, wherein the second direction is perpendicular to the first direction and parallel to a plane determined by the conveying direction and the axis of the first roller, and a circumferential surface of the cam is connected to a side of the first movable seat facing away from the second fixed seat;

a wrench connected to the cam for operation by the user to rotate the cam relative to the first manipulation device base.

According to the present application, the first pressing device is simple in structure and stable in performance.

Optionally, the first power transfer device comprises a first transmission shaft used to be connected to the first driving device so as to rotate under the driving of the first driving device;

the first manipulation device further comprises:

a first roller transmission device used to connect the first transmission shaft and the first roller so that the rotation direction of the first roller is the same as a rotation direction of the first transmission shaft, and

a second roller transmission device used to connect the first transmission shaft and the second roller so that the rotation direction of the second roller is opposite to the rotation direction of the first transmission shaft.

According to the present application, the first roller and the second roller are respectively driven by different transmission devices, so that even if one of the transmission devices fails, the roller of the fault party can also be passively rotated under the action of frictional force, without affecting the convey of the guidewire and thereby greatly ensuring the stable performance of the apparatus.

Optionally, the first roller transmission device comprises at least one first manipulation synchronous belt, the first transmission shaft and the first roller are both connected with a structure for connecting the first manipulation synchronous belt, and the slave-side execution apparatus is configured such that the first transmission shaft drives all the first rollers to rotate via the first manipulation synchronous belt.

According to the present application, the first roller transmission device has a simple structure, low cost and stable performance.

Optionally, the first manipulation device comprises N first rollers and N first manipulation synchronous belts, where N is an integer greater than or equal to 1, wherein,

each of the first rollers is connected to the first transmission shaft via one first manipulation synchronous belt; or

each of one part of the N first rollers is connected to the first transmission shaft via one first manipulation synchronous belt, and each of the other part of the N first rollers is connected to another first roller via one first manipulation synchronous belt.

According to the present application, the first roller transmission device is flexibly arranged.

Optionally, the first manipulation device comprises N first rollers and N first manipulation synchronous belts, where N is an integer greater than or equal to 1, wherein,

the first one of the N first rollers is connected to the first transmission shaft via one first manipulation synchronous belt, and each of the other first rollers is connected to the previous first roller via one first manipulation synchronous belt.

According to the present application, the first transmission shaft and all the first rollers are driven one by one in sequence, and thus the specification of each first roller may be unified to reduce costs.

Optionally, the first manipulation device further comprises:

a first additional roller disposed on the first movable seat, the first additional roller being configured to be rotatable about its axis relative to the first movable seat;

a second additional roller disposed on the second fixed seat and arranged opposite to the first additional roller so as to clamp the guidewire along with the first additional roller, the second additional roller being configured to be rotatable about its axis relative to the second fixed seat;

a first additional roller transmission device used to connect the first additional roller and the Nth one of the first rollers so that the first additional roller is rotated in the same direction as a rotation direction of the first transmission shaft under the driving of the Nth one of the first rollers; and

an encoder connected to the second additional roller and used to detect a rotation angle of the second additional roller.

According to the present application, when the first transmission shaft and all the first rollers are driven one by one in sequence, the rotation angle detected by the encoder may be used to detect whether the first manipulation synchronous belt is slipping or damaged.

Optionally, the second additional roller has a second additional roller axle;

the movable power device also comprises a third rotating shaft disposed on the movable power device, the third rotating shaft being used to be connected to the second additional roller axle and rotate synchronously with the second additional roller axle, wherein the encoder is disposed on the third rotating shaft.

According to the present application, the encoder is connected to the passive component via the third rotating shaft, so that the passive component may serve as a consumable.

According to the present application, the first additional roller transmission device has a simple structure, low cost and stable performance. The axis of the first additional roller is parallel to the axis of the first roller, and thus the design may be simplified.

Optionally, the second roller transmission device comprises:

a fourth transmission shaft disposed on the first manipulation device base;

a first transmission device for the second roller used to connect the first transmission shaft and the fourth transmission shaft so that the fourth transmission shaft is rotated under the driving of the first transmission shaft; and

a second transmission device for the second roller used to connect the fourth transmission shaft and the second roller so that the second roller is rotated under the driving of the fourth transmission shaft.

According to the present application, by designing the fourth transmission shaft so that the first transmission shaft drives the fourth transmission shaft to rotate, and the fourth transmission shaft then drives the second roller to rotate, it is convenient to make the rotation direction of the second roller different from the rotation direction of the first transmission shaft.

Optionally, one of the first transmission device for the second roller and the second transmission device for the second roller is configured to be driven by a synchronous belt, and the other of the first transmission device for the second roller and the second transmission device for the second roller is configured to be driven by a gear.

According to the present application, the synchronous belt transmission allows the rotation direction of the driven member the same as the rotation direction of the driving member, and the gear transmission allows the rotation direction of the driven member different from the rotation direction of the driving member, thereby allowing the rotation directions of the second roller and the first transmission shaft different.

Optionally, a component of the first roller transmission device for connecting the first transmission shaft and a component of the second roller transmission device for connecting the first transmission shaft are both synchronous belts.

According to the present application, the first transmission shaft is connected to the first roller transmission device and the second roller transmission device through the synchronous belt, which helps to simplify the design of the first transmission shaft.

Optionally, the first manipulation device comprises:

a driving roller member disposed on the first carrying device, the driving roller member comprising a driving roller and a driving gear coaxially connected, wherein the driving gear is in transmission connection with the first power transfer device so as to rotate about its own axis relative to the first carrying device under the action of the first power transfer device; and

a driven roller member disposed on the first carrying device, the driven roller member comprising a driven roller arranged opposite to the driving roller, the driven roller being configured to be rotatable about its own axis relative to the first carrying device and movable relative to the driving roller along a first direction, so as to be able to clamp or release the guidewire with the driving roller, wherein the first direction is perpendicular to a plane determined by the conveying direction and an axis of the driven roller.

According to the present application, among the pair of rollers clamping the guidewire, one is the driving roller that is rotated under the driving of the driving device, and the other is the driven roller that is rotated under the action of frictional force.

Optionally, the driven roller member further comprises:

a fixed frame disposed on the first carrying device, the fixed frame having a third guide rail extending along the first direction;

an extrusion frame disposed on the third guide rail and being movable along the third guide rail, the driven roller being disposed on the extrusion frame; and

a first elastic member connected between the fixed frame and the extrusion frame, the first elastic member extending along the first direction for providing a force that brings the extrusion frame close to the driving roller along the first direction.

According to the present application, the driven roller member is moved towards the driving roller member under the action of the first elastic member so that the guidewire is clamped by the driving roller along with the driven roller.

Optionally, the first manipulation device further comprises a manipulation rod assembly comprising:

a manipulation portion used for being operated by a user and exposed from the first carrying device;

a pivoting portion connected to the first carrying device and configured to be rotatable about its own axis relative to the first carrying device;

an extension portion connected between the pivoting portion and the manipulation portion so that the manipulation portion is capable of rotating synchronously with the pivoting portion about an axis of the pivoting portion;

a pushing portion provided to the extension portion for contacting the extrusion frame to enable the pushing portion to push the extrusion frame away from the driving roller along the first direction when the pivoting portion is rotated.

According to the present application, the manipulation rod assembly is used to move the driven roller member away from the driving roller member so that the guidewire may be taken and placed.

Optionally, a position of the first power transfer device corresponds to a position of the first power shaft, and the first power transfer device is engaged with the first power shaft, the first power transfer device comprises a transfer synchronous roller, and a synchronous belt transmission is formed between the first power transfer device and the first manipulation device.

According to the present application, the connection between the first power transfer device and the first power shaft is stable.

Optionally, the second transmission device comprises:

a second power shaft disposed on the movable power device and configured to be rotatable about its own axis relative to the movable power device;

a second transmission assembly disposed on the movable power device and connected between the second driving device and the second power shaft so that the second power shaft is rotated under the driving of the second driving device; and

a second power transfer device disposed on the first carrying device and connected between the second manipulation device and the second power shaft so that the second power shaft drives the second manipulation device to operate.

According to the present application, the second transmission device comprises the second power shaft, and the control of the second transmission device is simple.

Optionally, the first power shaft is arranged to penetrate through the movable power device, and an end of the first power shaft facing the first carrying device is in transmission connection with the first manipulation device;

the first transmission assembly is connected between the first driving device and an end of the first power shaft facing away from the first carrying device;

the second power shaft is arranged to penetrate through the movable power device, and an end of the second power shaft facing the first carrying device is in transmission connection with the second manipulation device;

the second transmission assembly is connected between the second driving device and an end of the second power shaft facing away from the first carrying device.

According to the present application, the first power shaft is stably connected to the first transmission assembly, and the second power shaft is stably connected to the second transmission assembly.

Optionally, the first carrying device comprises a functional manipulation seat for mounting the medical apparatus, the functional manipulation seat comprising a first side of the functional manipulation seat and a second side of the functional manipulation seat which are arranged opposite to each other, wherein the medical apparatus is detachably connected to the first side of the functional manipulation seat, and the functional manipulation seat is provided with an operation opening;

the second manipulation device comprises:

a functional gear rotatably disposed at the second side of the functional manipulation seat and at least partially exposed from the operation opening to the first side of the functional manipulation seat, so as to be able to form an engagement with the medical apparatus, and

a bevel gear coaxially connected with the functional gear and used to be connected to the second power transfer device.

According to the present application, the second manipulation device has a compact structure.

Optionally, the first carrying device further comprises:

a first bracket disposed on the first side of the functional manipulation seat;

a second bracket disposed on the first side of the functional manipulation seat and spaced apart from the first bracket along the conveying direction so as to support the medical apparatus along with the first bracket, the second bracket being configured to be movable relative to the functional manipulation seat along the conveying direction;

a second elastic member connected to the second bracket for applying a force to the second bracket to move the second bracket away from the first bracket along the conveying direction.

According to the present application, the supporting structure of the slave-side execution apparatus for carrying the medical apparatus is compact.

Optionally, a position of the second power transfer device corresponds to a position of the second power shaft, and the second power transfer device is engaged with the second power shaft, the second power transfer device comprises a transfer gear, and a gear engaging transmission is formed between the second power transfer device and the second manipulation device.

According to the present application, the second power transfer device is stably connected to the second power shaft.

Optionally, the main body power device further comprises:

a machine frame on which the main body driving device and the movable assembly are both provided; and

a first guide rail disposed on the machine frame and extending along the conveying direction;

wherein the movable assembly is connected to the first guide rail and is configured to move along the first guide rail under the driving of the main body driving device.

According to the present application, the first guide rail may cause the movable assembly to stably move along the conveying direction.

Optionally, the movable assembly comprises:

a movable frame configured to be moved along the first guide rail under the driving of the main body driving device, the movable frame having a second guide rail extending along the conveying direction;

a movable seat disposed on the second guide rail and being movable along the second guide rail, the movable power device being connected to the movable seat; and

a force sensor disposed between the movable seat and the movable frame to sense a force experienced by the movable seat when the catheter is conveyed.

According to the present application, the forces such as resistance and tension experienced by the catheter may be measured by the force sensor, allowing the user to better control the convey of the catheter.

Optionally, the slave-side execution apparatus further comprises a guide assembly disposed in front of the first manipulation device along the conveying direction;

the guide assembly comprises at least one pair of rollers arranged opposite to each other, the axes of the rollers are parallel to each other and the rollers are configured to be rotatable about their own axes, and one pair of rollers is used to jointly clamp the guidewire and/or the catheter.

According to the present application, the guide assembly has a guiding effect on the convey of the guidewire and/or the catheter.

Optionally, wherein the slave-side execution apparatus also comprises a second carrying device disposed on the main body power device and located in front of the first carrying device along the conveying direction, and the second carrying device has a guide assembly for guiding the guidewire and/or the catheter.

According to the present application, the guide assembly is arranged in front of the first manipulation device to guide the convey of the guidewire and/or the catheter.

Optionally, the second carrying device further comprises a supporting seat connected to the movable power device;

the guide assembly comprises:

a catheter guiding device disposed on the supporting seat so as to be able to clamp or release the guidewire and/or the catheter, and

a catheter supporting device disposed on the supporting seat and configured to allow the guidewire and/or the catheter to pass through to support the guidewire and/or the catheter.

According to the present application, the guide assembly has the dual functions of guiding and supporting.

Optionally, the catheter guiding device comprises:

a first guide roller disposed on the supporting seat and configured to be rotatable about its own axis relative to the supporting seat; and

a second guide roller disposed on the supporting seat and arranged opposite to the first guide roller, the second guide roller being configured to be rotatable about its own axis relative to the supporting seat and configured to be movable relative to the first guide roller along a first additional direction so as to be able to release or clamp the guidewire and/or the catheter with the first guide roller, wherein the first additional direction is perpendicular to a plane determined by the conveying direction and an axis of the second guide roller.

According to the present application, the guidance is performed by the catheter guiding device via a pair of rollers that clamp the guidewire and/or the catheter.

Optionally, the second carrying device further comprises:

a fixed block disposed on the supporting seat and having a guide groove extending along the first additional direction;

a pushing block at least partially extending into the guide groove, the second guide roller being provided on the pushing block;

a third elastic member disposed in the guide groove and connected between the fixed block and the pushing block, the third elastic member extending along the first additional direction to apply a force to the pushing block that causes the pushing block to move close to the first guide roller along the first additional direction.

According to the present application, the third elastic member enables the two guide rollers to clamp the guidewire and/or the catheter.

Optionally, the slave-side execution apparatus further comprises:

a second power assembly used to be installed beside a surgical bed and being immovable relative to the surgical bed, the second power assembly comprising a fourth driving device for providing a driving force; and

a second conveying assembly detachably connected to the second power assembly for conveying the guidewire and/or the catheter under the driving of the fourth driving device;

wherein the second power assembly is used to be installed in front of the main body power device along the conveying direction of the guidewire and/or the catheter.

According to the present application, the second conveying assembly is used to support and assist in the convey of the guidewire and/or the catheter at the front end.

Optionally, the second conveying assembly comprises:

a second conveying assembly base detachably connected to the second power assembly;

at least one third roller disposed on the second conveying assembly base, an axis of the third roller being not parallel to the conveying direction; and

at least one fourth roller disposed on the second conveying assembly base and arranged opposite to the third roller, so as to clamp the guidewire and/or the catheter along with the third roller;

wherein the third roller is used for rotating about its own axis under the driving of the fourth driving device.

According to the present application, the third roller may be the driving roller, and the fourth roller may be the driven roller.

Optionally, the second conveying assembly comprises:

a third roller assembly disposed on the second conveying assembly base and comprising at least one the third roller;

a fourth roller assembly comprises:

a fifth movable seat disposed on the second conveying assembly base and being movable relative to the second conveying assembly base along a first additional direction, wherein the first additional direction is perpendicular to the axis of the third roller, and

at least one the fourth roller disposed on the fifth movable seat, the fourth roller being arranged opposite to the corresponding third roller along the first additional direction; and

a position adjustment assembly connected to the fifth movable seat for driving the fifth movable seat to move along the first additional direction under the driving of a third driving device, so as to change a distance the fourth roller and the third roller.

According to the present application, the third roller and the fourth roller are arranged opposite each other to jointly clamp and convey the guidewire and/or the catheter, and the distance between the third roller and the fourth roller is adjustable so that the guidewire and/or the catheter of different diameters may be accommodated. The distance between the third roller and the fourth roller is controlled by the driving device, which allows the distance between them to be kept constant, i.e., a sufficient clamping force may be provided by the driving device. At the same time, the third roller from the fourth roller may be separated by the driving device, which facilitates the installation of the guidewire and/or the catheter.

Optionally, the position adjustment assembly comprises:

an adjusting gear disposed on the second conveying assembly base and used for rotating relative to the second conveying assembly base under the driving of the third driving device; and

an adjusting rack engaged with the adjusting gear, the adjusting rack extending along the first additional direction and connected to the fifth movable seat.

According to the present application, the position adjustment assembly is simple in structure, easy to control, and stable in performance.

Optionally, the third roller assembly comprises a plurality of the third rollers,

the third roller assembly is configured such that a part of the third rollers are used for rotating about their own axes under the driving of the fourth driving device and the other third rollers are rotated about their own axes under the driving of another third roller.

According to the present application, the connection between the third roller assembly and the fourth driving device is simple.

Optionally, the second conveying assembly further comprises a roller transmission assembly, a first end of the roller transmission assembly is connected to the third roller and a second end of the roller transmission assembly is connected to the fourth roller so that the third roller drives the fourth roller to rotate in an opposite direction at the same rotational speed,

wherein the roller transmission assembly is configured such that the second end of the roller transmission assembly is movable relative to the first end of the roller transmission assembly along the first additional direction, so as to adapt to the movement of the fifth movable seat along the first additional direction.

According to the present application, the fourth roller may also be a driving roller, thereby increasing the conveying capacity of the second conveying assembly. The fourth roller is driven by the third roller, and the connection between the second conveying assembly and the driving device may be reduced, so that the second conveying assembly is more convenient to use.

Optionally, the roller transmission assembly comprises N transmission gears that are engaged sequentially, N being an even number greater than or equal to 4,

wherein the first one of the transmission gears is disposed at the first end of the roller transmission assembly and is rotated synchronously with the third roller, the last one of the transmission gears is disposed at the second end of the roller transmission assembly and is rotated synchronously with the fourth roller, and the last one of the transmission gears is movable relative to the first one of the transmission gears along the first additional direction.

According to the present application, the roller transmission assembly is simple in structure, easy to control, and stable in performance.

Optionally, the third roller is configured as the same roller as the fourth roller, and the roller transmission assembly has a transmission ratio of 1:1.

According to the present application, the component specifications are unified, which is conducive to cost saving.

Optionally, the roller transmission assembly further comprises:

a first connecting arm comprising a first end of the first connecting arm and a second end of the first connecting arm arranged oppositely, the first one of the transmission gears being disposed on the first end of the first connecting arm; and

a second connecting arm comprising a first end of the second connecting arm and a second end of the second connecting arm arranged oppositely, the last one of the transmission gears being disposed on the second end of the second connecting arm,

wherein the first end of the second connecting arm is rotatably connected to the second end of the first connecting arm about a third axis, the third axis is parallel to an axis of the third roller, and N transmission gears are disposed on the first connecting arm and the second connecting arm.

According to the present application, the way of realizing the extension and retraction of the roller transmission assembly in the first additional direction is simple, easy to implement and stable in performance.

Optionally, the third axis coincides with an axis of one transmission gear other than the first one of the transmission gears and the last one of the transmission gears.

According to the present application, the distance between the adjacent transmission gears is always kept constant, which is conducive to the good engagement of all transmission gears at all times.

Optionally, the fourth roller assembly comprises a plurality of the fourth rollers,

the fourth roller assembly is configured such that a part of the fourth rollers are used for rotating about their own axes under the driving of the third roller and the other fourth rollers are rotated about their own axes under the driving of another fourth roller.

According to the present application, the roller transmission assembly only needs to connect a part of the fourth roller, and the connection between the roller transmission assembly and the fourth roller assembly is simplified.

Optionally, the fourth roller assembly further comprises:

a plurality of second rotating shafts disposed corresponding to the fourth roller and coaxially connected with the corresponding fourth roller, wherein a part of the second rotating shafts are used for rotating about their own axes under the driving of the third roller; and

at least one second transmission belt, each of which is connected with two second rotating shafts.

According to the present application, the method by which the second conveying assembly makes all the fourth rollers rotate simultaneously is simple, stable in performance and easy to implement.

Optionally, the third roller assembly comprises a plurality of the third rollers, and the fourth roller assembly comprises a plurality of the fourth rollers,

wherein the Pth one of the third rollers is used for rotating about its own axis under the driving of the fourth driving device, the first end of the roller transmission assembly is connected to the Qth one of the third rollers, the second end of the roller transmission assembly is connected to the Qth one of the fourth rollers, and P is not equal to Q.

According to the present application, the fourth driving device and the roller transmission assembly are respectively connected with different third rollers, which can avoid mutual interference and is also conducive to simplifying the connection structure. The roller transmission assembly is nearby connected to the third and fourth rollers which are opposite to each other to save material and avoid the interference between the rollers.

Optionally, the second conveying assembly further comprises a biasing member for applying a force to the fifth movable seat to move away from the third roller along the first additional direction.

According to the present application, the biasing member assists the position adjustment assembly to hold the fourth roller in a stable position with respect to the third roller. The biasing member also facilitates opening the channel between the fourth and third rollers, allowing for the installation of the guidewire and/or the catheter. The biasing member also makes it difficult for the fourth roller to contact the third roller, which helps to protect the fourth roller, the third roller, the guidewire and the catheter.

A second aspect of the present application provides a medical system comprising:

a first supporting frame used to be disposed beside a surgical bed;

the slave-side execution apparatus of any one of the aforesaid technical solutions, wherein the main body power device is connected to the first supporting frame; and

a medical apparatus detachably connected to the first carrying device.

According to the present application, the slave-side execution apparatus of the slave-side may be controlled to operate by the doctors in the main control room, thereby preventing the doctors form being exposed to radiation of the ray. The components of the slave-side execution apparatus for contacting the guidewire and/or the catheter are passive components (without the need to connect to a power source), may be configured as consumables, are easy to install, and infection may be avoided.

Optionally, the first supporting frame is configured as a robotic arm, the robotic arm being configured to be movable relative to the surgical bed in at least one spatial dimension.

According to the present application, the first supporting frame is configured as the robotic arm so that the position and angle of the slave-side execution apparatus may be adjusted.

A third aspect of the present application provides a medical system, comprising:

a first supporting frame used to be disposed beside a surgical bed;

a second supporting frame used to be disposed beside the surgical bed and located in front of the first supporting frame along the conveying direction of the guidewire and/or the catheter;

the slave-side execution apparatus of any one of the aforesaid technical solutions comprising the second conveying assembly, wherein the main body power device is connected to the first supporting frame, and the second power assembly is connected to the second supporting frame; and

a medical apparatus detachably connected to the first carrying device.

According to the present application, the slave-side execution apparatus of the slave-side may be controlled to operate by the doctors in the main control room, thereby preventing the doctors form being exposed to radiation of the ray. The components of the slave-side execution apparatus for contacting the guidewire and/or the catheter are passive components (without the need to connect to a power source), may be configured as consumables, are easy to install, and infection may be avoided.

Optionally, the first supporting frame and/or the second supporting frame is configured as a robotic arm, the robotic arm being configured to be movable relative to the surgical bed in at least one spatial dimension.

According to the present application, the first supporting frame and/or the second supporting frame is configured as the robotic arm so that the position and angle of the slave-side execution apparatus may be adjusted.

Optionally, the medical system further comprises:

a master-side operating apparatus; and

a processor module signal-connected to both the master-side operating apparatus and the slave-side execution apparatus.

According to the present application, the slave-side execution apparatus may be controlled by the doctors at the master-side operating apparatus.

### BRIEF DESCRIPTION OF THE DRAWINGS

The following drawings of the present application are used as a part of the present application for understanding the present application. The drawings show the embodiments of the present application and the descriptions thereof, and are used to explain the principles of the present application.

In the drawings:

FIG. 1 is a perspective schematic view of a medical system according to a first embodiment of the present application;

FIG. 2 is a perspective exploded schematic view of a slave-side execution apparatus according to the first embodiment of the present application;

FIG. 3 is an exploded schematic view of a rear consumable box shown in FIG. 2;

FIGS. 4 and 5 are schematic views of some components of the slave-side execution apparatus shown in FIG. 2, wherein a main body power device and a movable power device are shown;

FIG. 6 is an exploded schematic view of some components of the slave-side execution apparatus shown in FIG. 2, wherein a main body power device and a movable power device are shown;

FIG. 7 is an exploded schematic view of a carrying base shown in FIG. 3, wherein a snapping assembly is shown;

FIGS. 8 and 9 are schematic views of an internal structure of the main body power device shown in FIG. 2;

FIG. 10 is an exploded schematic view of a movable assembly shown in FIG. 8;

FIG. 11 is a schematic view of an internal structure of the movable power device shown in FIG. 6;

FIG. 12 is another exploded schematic view of the carrying base shown in FIG. 3, wherein a first power transfer device and a second power transfer device are shown;

FIG. 13 is a schematic view of a first manipulation device shown in FIG. 2 being removed from a first carrying device;

FIG. 14 is a schematic view of the first manipulation device shown in FIG. 2 being placed in the first carrying device;

FIG. 15 is a perspective exploded schematic view of the first manipulation device shown in FIG. 2;

FIG. 16 is a schematic bottom view of the first manipulation device shown in FIG. 2;

FIG. 17 is another perspective exploded schematic view of the first manipulation device shown in FIG. 2;

FIG. 18 is yet another perspective exploded schematic view of the first manipulation device shown in FIG. 2;

FIG. 19 is a schematic bottom view of some components of the first manipulation device shown in FIG. 2, wherein a first movable roller group has been pressed by a pressing device to a first fixed roller group;

FIG. 20 is a schematic bottom view of some components of the first manipulation device shown in FIG. 2, wherein the first movable roller group is not pressed by the pressing device to the first fixed roller group;

FIG. 21 is a perspective schematic view of the first movable roller group of the first manipulation device shown in FIG. 2;

FIG. 22 is a perspective schematic view of the first fixed roller group of the first manipulation device shown in FIG. 2;

FIG. 23 is a schematic view of some components of the rear consumable box shown in FIG. 3, wherein a functional manipulation seat and a second manipulation device are shown;

FIG. 24 is an exploded schematic view of the components shown in FIG. 23;

FIG. 25 is a schematic view of a specific example of the movable assembly shown in FIG. 8;

FIG. 26 is an exploded schematic view of the movable assembly shown in FIG. 25;

FIG. 27 is a structural block view of a medical system according to a specific embodiment of the present application;

FIG. 28 is a schematic view of a second power assembly and a second conveying assembly of the slave-side execution apparatus according to the specific embodiment of the present application;

FIG. 29 is a perspective exploded schematic view of the components shown in FIG. 28;

FIG. 30 is a perspective exploded schematic view of the second conveying assembly shown in FIG. 28;

FIG. 31 is a perspective exploded schematic view of an internal structure of the second conveying assembly shown in FIG. 30;

FIG. 32 is a side view of the internal structure of the second conveying assembly shown in FIG. 30, wherein a fourth roller is close to a third roller;

FIG. 33 is a side view of the internal structure of the second conveying assembly shown in FIG. 30, wherein the fourth roller is away from the third roller;

FIG. 34 is a schematic view of a roller transmission assembly of the second conveying assembly shown in FIG. 30;

FIG. 35 is an overall schematic view of a slave-side execution apparatus according to a second embodiment of the present application;

FIGS. 36 and 37 are schematic views of some components of the slave-side execution apparatus shown in FIG. 35, wherein a main body power device and a movable power device are shown;

FIG. 38 is an exploded schematic view of some components of the slave-side execution apparatus shown in FIG. 35, wherein the main body power device and the movable power device are shown;

FIGS. 39 and 40 are schematic views of an internal structure of the main body power device of the slave-side execution apparatus shown in FIG. 35;

FIG. 41 is an exploded schematic view of a movable assembly shown in FIG. 39;

FIGS. 42 and 43 are schematic views of an internal structure of the movable power device shown in FIG. 35;

FIG. 44 is an overall schematic view of the first carrying device shown in FIG. 35;

FIG. 45 is an exploded schematic view of the first carrying device shown in FIG. 35;

FIG. 46 is an exploded schematic view of the carrying base shown in FIG. 45, wherein a snapping assembly is shown;

FIG. 47 is another exploded schematic view of the carrying base shown in FIG. 45, wherein a first power transfer device and a second power transfer device are shown;

FIGS. 48 to 50 are schematic views of a first manipulation device shown in FIG. 45;

FIG. 51 is a schematic view of a driven roller member shown in FIG. 49;

FIG. 52 is an exploded schematic view of the driven roller member shown in FIG. 51;

FIGS. 53 and 54 are schematic views of a functional manipulation seat shown in FIG. 45;

FIG. 55 is an exploded schematic view of a functional manipulation seat shown in FIG. 45;

FIGS. 56 and 57 are schematic views of a second carrying device shown in FIG. 35; and

FIG. 58 is a partial exploded schematic view of the second carrying device shown in FIG. 56.

### DETAILED DESCRIPTION

In the following description, numerous specific details are set forth in order to provide a more thorough understanding of the present application. However, it is obvious to those skilled in this art that the present application may be implemented without one or more of these details. In other examples, some technical features well-known in this art are not described in order to avoid confusion with the present application.

In order to thoroughly understand the present application, a detailed description will be provided in the following description. It should be understood that these embodiments are provided to make the disclosure of the present application thorough and complete and the concept of these exemplary embodiments be completely delivered to those of ordinary skill in the art. Obviously, the implementing of the embodiments of the present application is not limited to the specific details familiar to those skilled in the art. The preferred embodiments of the present application are described in detail as follows. However, in addition to these detailed descriptions, the present application may have other embodiments.

The ordinals such as “first” and “second” recited in the present application are merely identifiers and do not have any other meaning, such as a specific order and the like. Moreover, for example, the term “first component” itself does not imply the presence of “second component”, and the term “second component” itself does not imply the presence of “first component.” The use of the terms “first”, “second” and “third” does not indicate any order, and these terms may be interpreted as names.

It should be noted that the terms “up”, “down”, “front”, “back”, “left”, “right”, “inside”, “outside” and similar expressions used herein are for illustrative purposes only and are not restrictive.

The present application provides a slave-side execution apparatus (slave-side conveying device) of a surgical robot for conveying a guidewire and/or a catheter, and a medical system (surgical robot system) using the slave-side execution apparatus.

The exemplary embodiments of the present application will now be explained in further details with reference to the accompanying drawings.

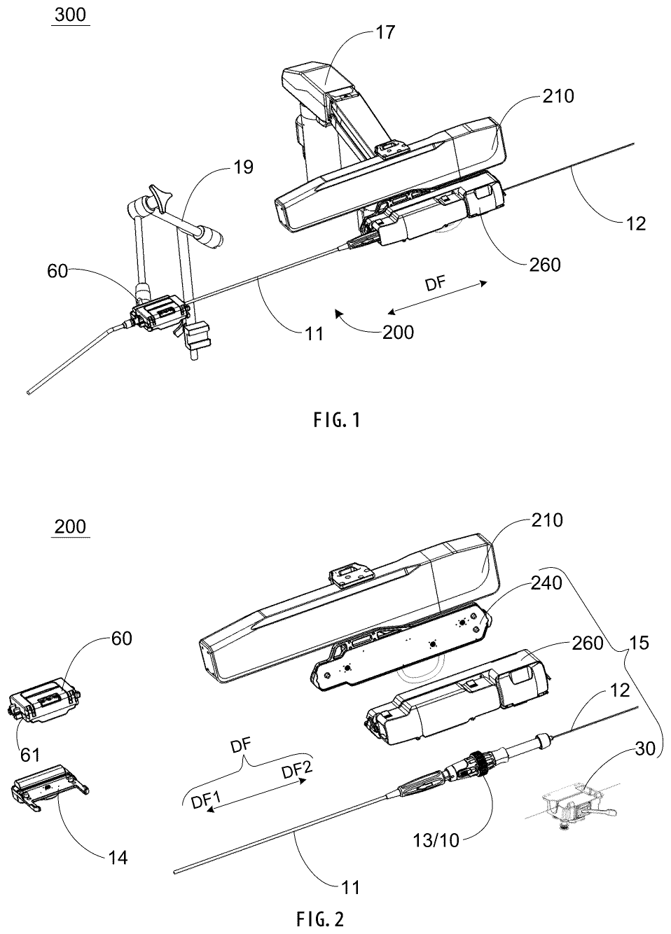

As shown in FIGS. 1 and 2, in a first embodiment of the present application, a medical system 300 includes a first supporting frame 17, a slave-side execution apparatus 200 according to a preferred embodiment of the present application, and a medical apparatus 10. Optionally, the medical system 300 also includes a second supporting frame 19. The second supporting frame 19 is located in front of the first supporting frame 17 along a conveying direction DF of a guidewire 12 and/or a catheter 11. During use, the first supporting frame 17 and the second supporting frame 19 are arranged beside a surgical bed for installing the slave-side execution apparatus 200. For example, the first supporting frame 17 and the second supporting frame 19 may be configured as robotic arms, such as passive robotic arms. The slave-side execution apparatus 200 is used for conveying the guidewire 12 and/or the catheter 11. The robotic arm has joints and may move in multiple spatial dimensions relative to the surgical bed, so that the position and angle of the slave-side execution apparatus 200 may be adjusted.

In the present application, the direction in which the guidewire 12 and/or the catheter 11 enters the blood vessel is the front (front end), and the direction in which the guidewire 12 and/or the catheter 11 exits the blood vessel is the back (rear end).

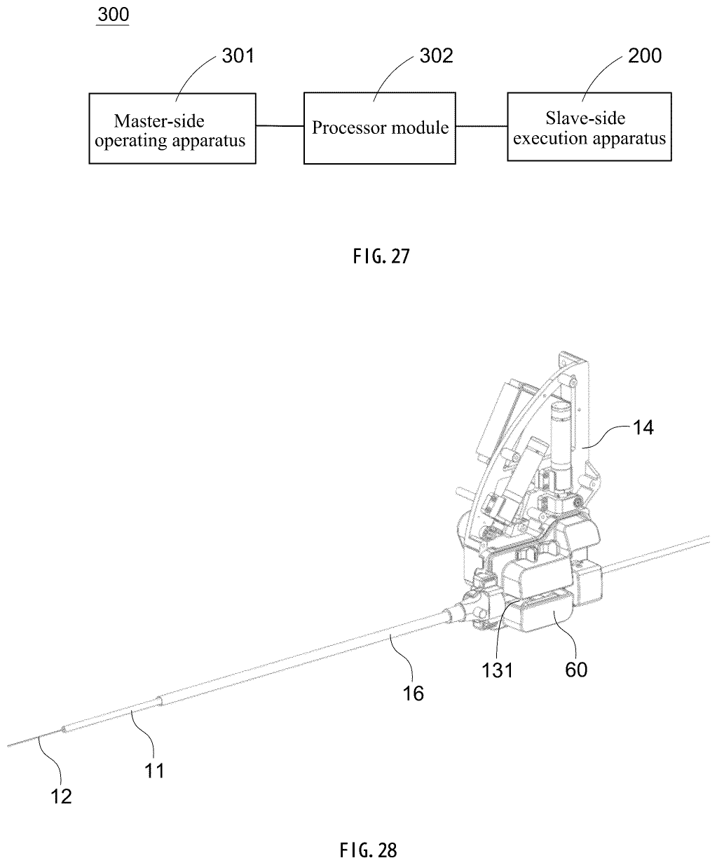

Optionally, the medical system 300 further includes a master-side operating apparatus 301 and a processor module 302 (see FIG. 27). The master-side operating apparatus is arranged in the doctor's working room for manual operation by the doctor. For example, the master-side operating apparatus may be a doctor's operating table. The processor module is signal-connected to both the master-side operating apparatus and the slave-side execution apparatus 200. The processor module may be a control device such as a central processing unit or a computer. After receiving a command signal from the master-side operating apparatus, the processor module processes the command signal and then sends it to the slave-side execution apparatus 100, or directly sends the command signal to the slave-side execution apparatus 200. After receiving the signal transmitted by the processor module, the slave-side execution apparatus 200 performs a corresponding action in response to an action of the master-side operating apparatus, so that the doctor may control the slave-side execution apparatus 200 at the master-side operating apparatus.

As shown in FIGS. 1 and 2, in a preferred embodiment, the slave-side execution apparatus 200 includes a main body power device 210, a movable power device 240, a first carrying device 260 and a first manipulation device 30.

Wherein, the main body power device 210 is used to be installed beside the surgical bed and is immovable relative to the surgical bed. For example, the main body power device 210 may be installed to the first supporting frame 17 so that it is immovable relative to the surgical bed. The main body power device 210 includes a main body driving device for providing a driving force. The movable power device 240 is connected to the main body power device 210 and is used to move forward and backward relative to the main body power device 210 along the conveying direction DF of the guidewire 12 and/or the catheter 11 under the driving of the main body driving device. The movable power device 240 includes a first driving device for providing a driving force. The first carrying device 260 is used to carry the medical apparatus 10 that has the catheter 11. The first carrying device 260 is detachably connected to the movable power device 240 and is synchronously movable with the movable power device 240 along the conveying direction DF relative to the main body power device 210. The first manipulation device 30 is connected to the first carrying device 260 and is used to control the convey of the guidewire 12 under the driving of the first driving device, i.e., to control the guidewire 12 to move forward and backward relative to the first carrying device 260 along the conveying direction DF. The first manipulation device 30 is located at a rear end of the first carrying device 260 along the conveying direction DF, or in other words, the first manipulation device 30 is disposed at a rear of the medical apparatus 10.

It may be understood that the conveying direction DF is a bidirectional direction, which includes a first conveying direction DF1 (forward direction) and a second conveying direction DF2 (backward direction), and the forward direction DF1 is opposite to the backward direction DF2.

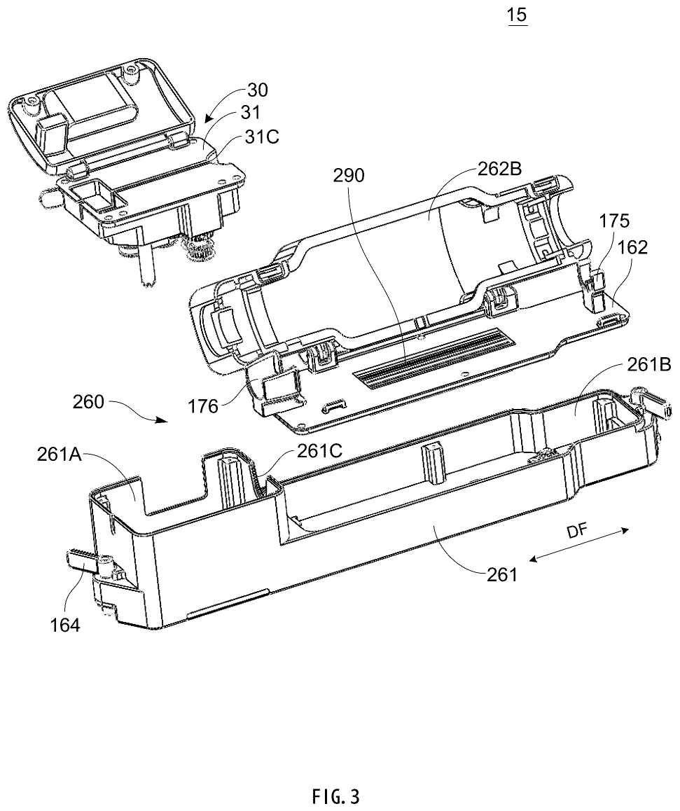

The movable power device 240 includes a second driving device for providing a driving force. The medical apparatus 10 is detachably connected to the first carrying device 260. As shown in FIG. 3, the first carrying device 260 includes a second manipulation device 290 that is used to contact the medical apparatus 10. The second manipulation device 290 is connected to the second driving device to manipulate the medical apparatus 10 to perform a medical function under the driving of the second driving device.

The medical apparatus 10 is, for example, configured to convey an artificial valve, which may provide a catheter 11. For example, the medical apparatus 10 is a functional handle of a valve conveyor, which is used for performing functions such as releasing or retrieving the artificial valve and to achieve relative movement and bending of the inner and outer shaft sheaths. As shown in FIG. 2, the medical apparatus 10 has at least one set of medical function mechanisms 13. When the medical function mechanisms 13 are in operation, the intended medical functions are performed by the medical apparatus 10. For example. The medical function mechanism 13 is configured as an operating gear 13 disposed on the periphery of the medical apparatus 10. When the operating gear 13 is rotated, the mechanism inside the medical apparatus 10 is driven by the operating gear 13 to operate, so as to achieve the intended medical functions.

Optionally, the slave-side execution apparatus 200 further includes a second power assembly 14 and a second conveying assembly 60. The second power assembly 14 is used to be installed beside the surgical bed and is immovable relative to the surgical bed. For example, the second power assembly 14 may be mounted to the second supporting frame 19 so that it is immovable relative to the surgical bed. The second power assembly 14 includes a fourth driving device for providing a driving force. The second conveying assembly 60 is detachably connected to the second power assembly 14, and is used for conveying the guidewire 12 and/or the catheter 11 under the driving of the fourth driving device.

As shown in FIG. 3, the first carrying device 260 is configured in the shape of a box. The medical apparatus 10 and the first manipulation device 30 are arranged in the first carrying device 260. The first carrying device 260 includes a carrying base 261, a functional manipulation seat 162 and a flip cover 262A. Wherein the carrying base 261 is used to be connected to the movable power device 240. The carrying base 261 includes a first part 261A of the carrying base and a second part 261B of the carrying base. The first part 261A of the carrying base and the second part 261B of the carrying base are arranged along the conveying direction DF. The first part 261A of the carrying base and the second part 261B of the carrying base are both configured in the shape of a box. The first part 261A of the carrying base is used to accommodate the first manipulation device 30. The first manipulation device 30 may be stably maintained in the first part 261A of the carrying base. The second part 261B of the carrying base is used to accommodate the second manipulation device 290. Specifically, the second manipulation device 290 is disposed on the functional manipulation seat 162 that covers the second part 261B of the carrying base. The medical apparatus 10 is mounted on the functional manipulation seat 162. For example, the functional manipulation seat 162 is provided with brackets 175 and 176 for mounting the medical apparatus 10. The flip cover 262B is pivotally connected to the functional manipulation seat 162 to cover the functional manipulation seat 162 and thereby cover the medical apparatus 10.

The first part 261A of the carrying base is provided with a notch 261C so that the guidewire located in the first part 261A of the carrying base may pass through the notch 261C and extend to the medical apparatus 10 corresponding to the position of the second part 261B of the carrying base.

In the present application, both the first carrying device 260 and the first manipulation device 30 do not include a driving device or a driving device for providing a driving force and are passive components. Therefore, the first carrying device 260 and the first manipulation device 30 may be used as consumables, both of which may be collectively referred to as a rear consumable box 15. The medical apparatus 10 is also a passive component, which is used for installation in the first carrying device 260 for use and may also be considered as a part of the rear consumable box 15. Similarly, the second conveying assembly 60 is also a passive component, which serve as a consumable and may be referred to as a front consumable box.

During the surgery, firstly, the guidewire 12 is fed into the blood vessel by the first manipulation device 30 under the action of the first driving device and reaches a desired position. The catheter 11 is sleeved on the guidewire 12 from the rear end so that the guidewire 12 runs through the catheter 11. Under the action of the main body driving device, the movable power device 240 and the first carrying device 260 are moved forward along the conveying direction DF, thereby driving the medical apparatus 10 to move forward, i.e., driving the catheter 11 to move forward into the blood vessel. That is, the catheter 11 enters the blood vessel under the support and guidance of the guidewire 12. At this time, the rear consumable box 15 also drives the first manipulation device 30 to move forward. In order to prevent the guidewire 12 from deviating from the desired position, while the catheter 11 is being conveyed, the first driving device acts on the first manipulation device 30 so that the guidewire 12 is moved backward along the conveying direction DF by the first manipulation device 30. It may be understood that the medical system 300 includes a control module (e.g., disposed in the master-side operating apparatus) that controls the main body driving device in cooperation with the first driving device such that only the catheter 11 is moved forward, but the guidewire 12 remains relatively stationary. The second conveying assembly 60 is located relatively at the front end and functions to support and assist in the convey of the guidewire 12 and/or the catheter 11. When the catheter 11 is conveyed in place, the second driving device controls the medical apparatus 10 to operate to complete the release of the artificial valve.

Optionally, at least one selected from the main body driving device, the first driving device, the second driving device and the fourth driving device includes a motor. Alternatively, the main body driving device, the first driving device, the second driving device and the fourth driving device all adopt the motor to output a driving force.

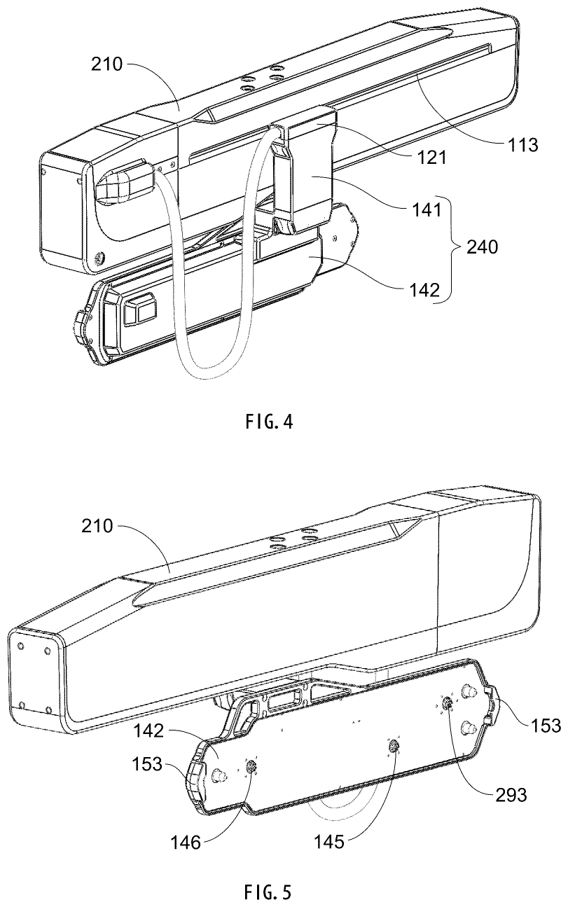

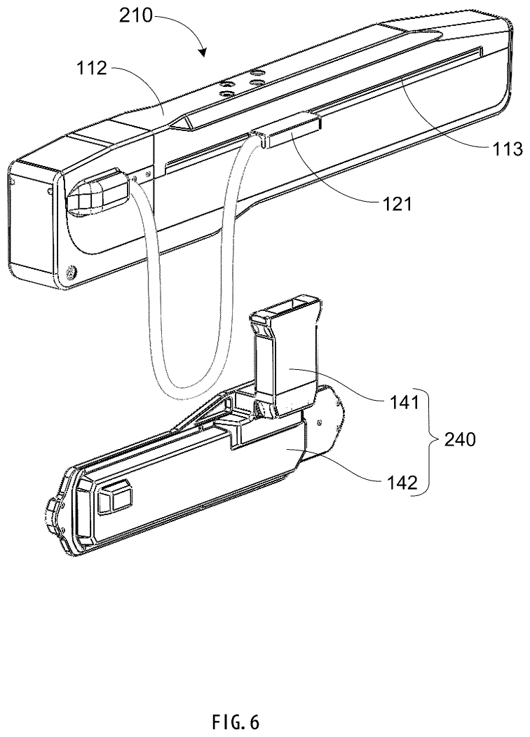

As shown in FIGS. 4 to 6, the movable power device 240 is connected to the main body power device 210 and is used to be moved forward and backward relative to the main body power device 210 along the conveying direction DF of the guidewire 12 and/or the catheter 11 under the driving of the main body driving device.

The main body power device 210 includes a housing 112 inside which a movable assembly and the main body driving device are provided. The movable assembly is configured to move forward and backward relative to the main body power device 210 along the conveying direction DF under the driving of the main body driving device, wherein the movable power device 240 is connected to the movable assembly. Specifically, the housing 112 has a long hole 113 extending along the conveying direction DF. The movable assembly has a protrusion 121 extending out of the housing 112 from the long hole 113. The movable power device 240 is connected to the protrusion 121. For example, the movable power device 240 includes a connecting frame 141 and a base 142. One end of the connecting frame 141 is connected to the protrusion 121, and the other end of the connecting frame 141 is connected to the base 142. Wherein the first carrying device 260 is detachably connected to the base 142.

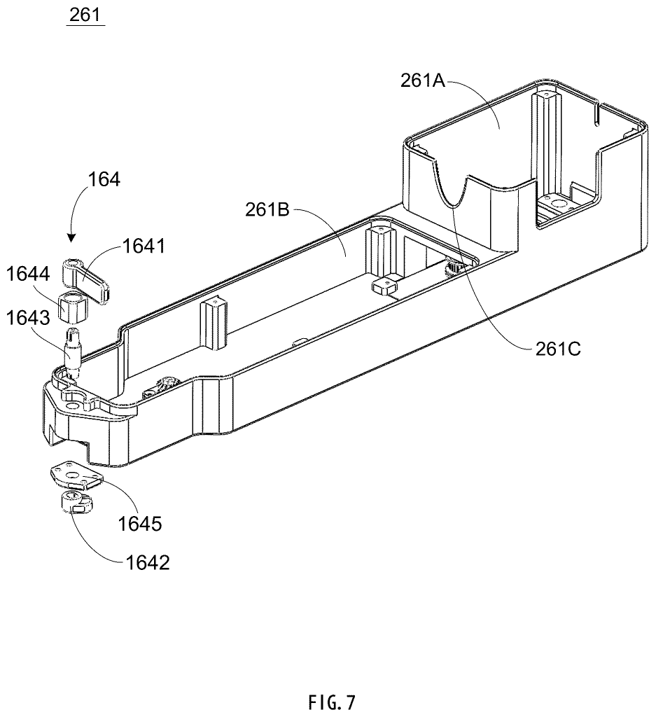

Specifically, the carrying base 261 of the first carrying device 260 is detachably connected to the base 142. As shown in FIG. 7, a snapping assembly 164 is provided at an end of the carrying base 261. A buckle seat 153 is provided at an end of the base 142 of the movable power device 240 (as shown in FIG. 5). The snapping assembly 164 is configured to connect to the buckle seat 153 in a snap-fit manner so that the carrying base 261 is detachably connected to the base 142.

For example, the snapping assembly 164 includes a knob 1641, a buckle 1642, and a rotating shaft 1643. The rotating shaft 1643 is disposed at the end of the carrying base 261 and penetrates through the carrying base 261. The knob 1641 is used for user operation. The knob 1641 is disposed at an end of the rotating shaft 1643 facing away from the base 142, and is synchronously rotatable with the rotating shaft 1643 about an axis of the rotating shaft 1643. The buckle 1642 is disposed at an end of the rotating shaft 1643 facing the base 142, and is synchronously rotatable with the rotating shaft 1643 about the axis of the rotating shaft 1643. When the knob 1641 is rotated by the user, the buckle 1642 is rotated synchronously with the knob 1641 through the rotating shaft 1643, and for example, it may be rotated into a groove of the buckle seat 153, so that the carrying base 261 is connected to the base 142 in a snap-fit manner. When the buckle 1642 is rotated out of the groove of the buckle seat 153, the carrying base 261 is disconnected from the base 142.

Optionally, the snapping assembly 164 further includes a damping member 1644 and a cover plate 1645. The damping member 1644 is sleeved on the rotating shaft 1643 and embedded in the carrying base 261. That is, the damping member 1644 is arranged between the rotating shaft 1643 and the carrying base 261. The cover plate 1645 is connected to the carrying base 261 to limit the position of the damping member 1644 and the rotating shaft 1643.

The first carrying device 260 may also be connected to the movable power device 240, for example, by magnetic attraction, suspension, etc.

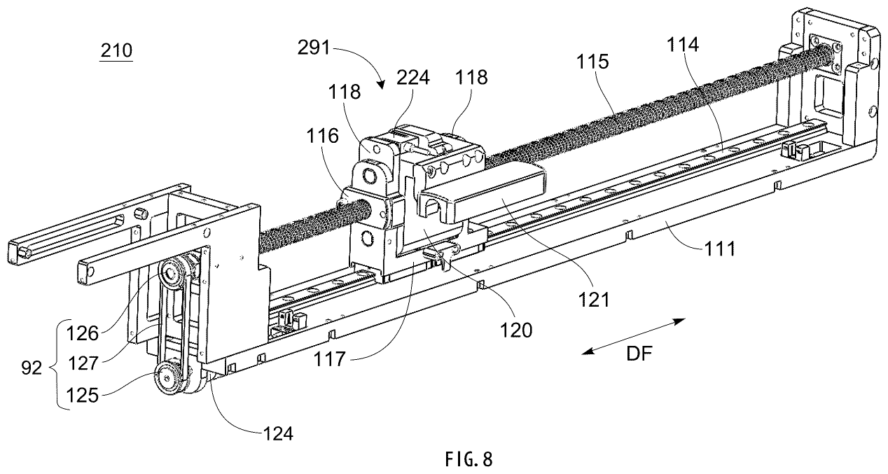

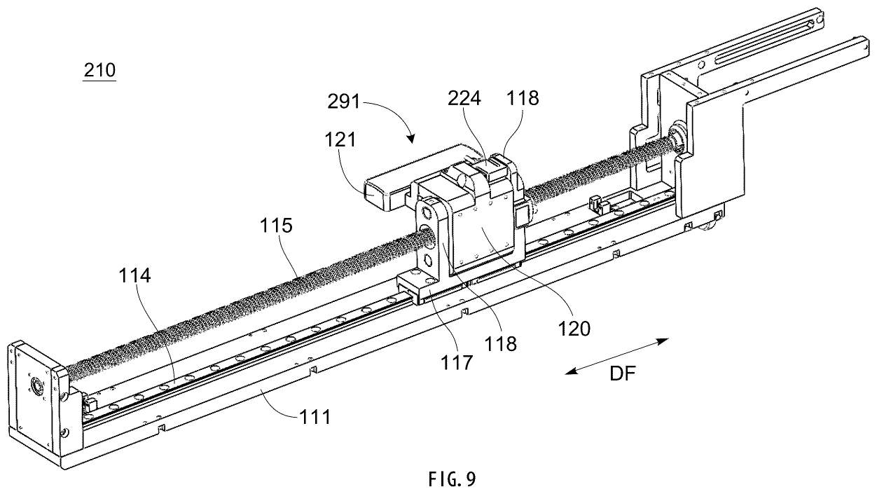

As shown in FIGS. 8 and 9, the main body power device 210 also includes a machine frame 111. The housing 112 covers the machine frame 111. The main body driving device 124 and the movable assembly 291 are both disposed on the machine frame 111. The main body power device 210 further includes a first guide rail 114, a lead screw pair and a main body transmission assembly 92. The first guide rail 114 is disposed on the machine frame 111. The first guide rail 114 extends along the conveying direction DF. The lead screw pair includes a lead screw 115 and a lead screw nut 116. The lead screw 115 is disposed on the machine frame 111 and also extends along the conveying direction DF. The main body transmission assembly 92 is connected between the main body driving device 124 and the lead screw 115 so that the lead screw 115 is rotatable about its own axis under the driving of the main body driving device 124. The movable assembly 291 is connected to the lead screw nut 116 and the first guide rail 114, and is configured to be movable along the first guide rail 114. The main body driving device 124 is in transmission connection with the lead screw 115 so that the lead screw nut 116 may be driven to move along an extension direction of the lead screw 115, and the movable assembly 291 is thereby driven to move along the extension direction of the lead screw 115. Furthermore, the movable assembly 291 drives the movable power device 240 to move through its protrusion 121.

Optionally, the bottom of a movable frame 117 is connected to the first guide rail 114.

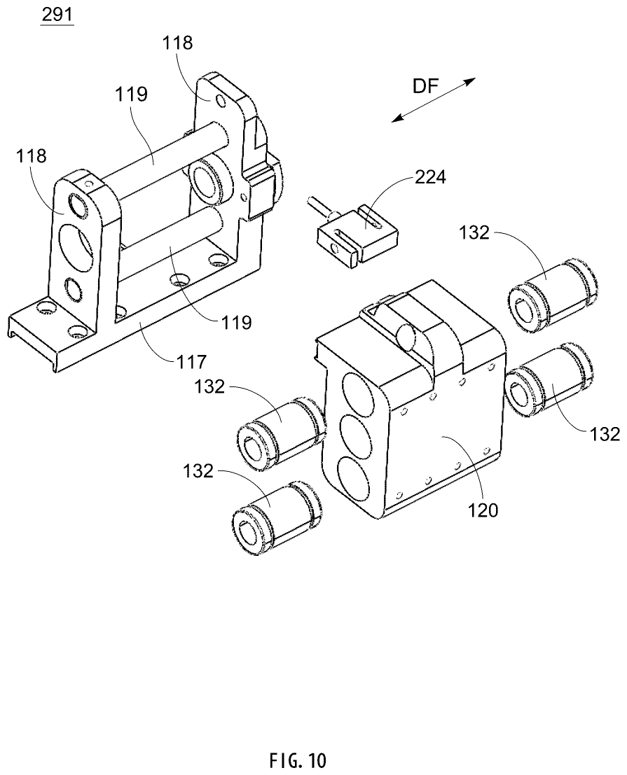

As shown in FIGS. 8 to 10, the movable assembly 291 includes a movable frame 117, a movable seat 120 and a tension-compression sensor 224. The movable frame 117 is fixedly connected to the lead screw nut 116. The movable frame 117 has two end walls 118 spaced apart along the conveying direction DF and a second guide rail 119 disposed between the two end walls 118. The second guide rail 119 extends along the conveying direction DF. The movable frame 117, for example, has two second guide rails 119 extending parallel to each other in the same horizontal plane. The movable seat 120 is disposed on the second guide rail 119 and is movable along the second guide rail 119. For example, the second guide rail 119 extends through the movable seat 120. Wherein the movable seat 120 is preferably disposed on the second guide rail 119 via linear bearing(s) 132. In other words, the linear bearing(s) 132 is sleeved outside the second guide rail 119, and the movable seat 120 is disposed outside the linear bearing(s) 132. Moreover, the lead screw 115 preferably integrally passes through the movable frame 117 and the movable seat 120, and preferably does not interfere with the movable frame 117 and the movable seat 120. The protrusion 121 is disposed on the movable seat 120. The tension-compression sensor 224 is disposed between the movable seat 120 and one of the two end walls 118, and abuts against both the movable seat 120 and the one of the two end walls 118 to sense the force experienced by the movable seat 120 when the catheter 11 is conveyed, i.e., to sense the conveying resistance.

It may be understood by those skilled in the art that the tension-compression sensor 224 is configured to be tension-compression integrated, and may read the force in the positive and negative directions. The tension-compression sensor 224 is electrically connected to the control module so that the doctor may be aware of the resistance experienced by the medical apparatus 10 in conveying the guidewire and make timely adjustments. After the components of the slave-side execution apparatus 200 are assembled and the main body power device 210 is mounted to the supporting frame 17 (at this point, the medical apparatus 10 has been mounted on the first carrying device 260), the measured value of the tension-compression sensor 224 needs to be set to zero first, and then it may be directly used to reflect the conveying resistance.

The slave-side execution apparatus 200 further includes a first transmission device 21 connected between the first driving device and the first manipulation device 30 and used to transfer the driving force of the first driving device to the first manipulation device 30.

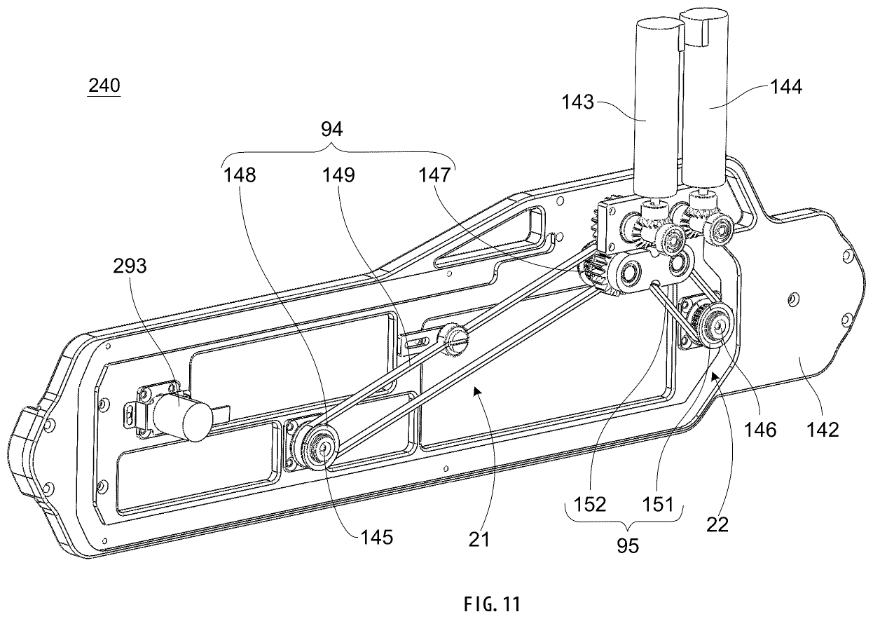

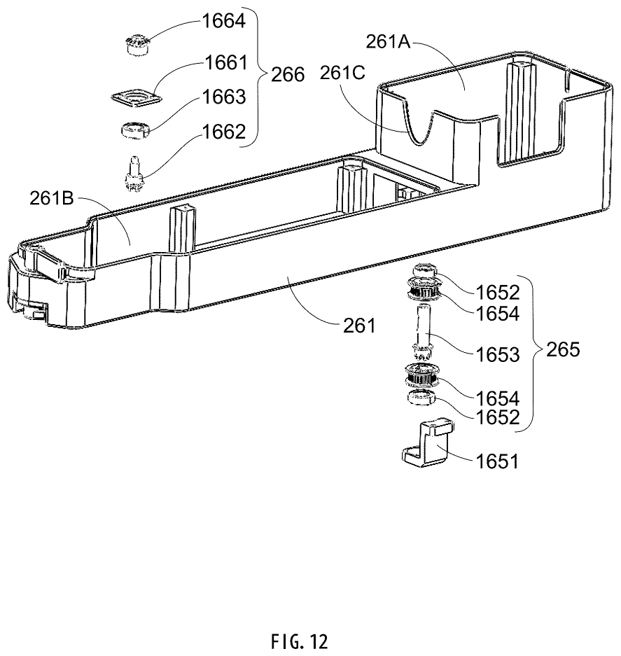

As shown in FIGS. 5, 11 and 12, the first transmission device 21, for example, includes a first power shaft 145, a first transmission assembly 94 and a first power transfer device 265.

The first power shaft 145 is connected to the first manipulation device 30 and disposed on the movable power device 240. The first power shaft 145 is configured to be rotatable about its own axis relative to the movable power device 240. The first power shaft 145 is, for example, disposed on the base 142 and penetrates through the base 142. An end of the first power shaft 145 facing the first carrying device 260 is in transmission connection with the first manipulation device 30.

The first driving device 143 is, for example, disposed on the connecting frame 141 of the movable power device 240. The first transmission assembly 94 is disposed on the movable power device 240, e.g., on the base 142. The first transmission assembly 94 is connected between the first driving device 143 and an end of the first power shaft 145 facing away from the first carrying device 260, so that the first power shaft 145 is rotatable under the driving of the first driving device 143. For example, the first transmission assembly 94 includes a first driving roller 147, a first driven roller 148, and a first synchronous belt 149. The first driving roller 147 is connected to a driving end of the first driving device 143. The first driven roller 148 is connected to the end of the first power shaft 145 facing away from the first carrying device 260. The first synchronous belt 149 is connected between the first driving roller 147 and the first driven roller 148 to transfer the power generated by the first driving device 143 to the first power shaft 145 so as to cause it to rotate.

The first power transfer device 265 is disposed on the first carrying device 260, e.g., in the second part 261B of the carrying base. The first power transfer device 265 is connected between the first manipulation device 30 and the first power shaft 145, so that the first manipulation device 30 is driven to operate by the first power shaft 145. The position of the first power transfer device 265 corresponds to the position of the first power shaft 145, and the first power transfer device 265 is engaged with the first power shaft 145. Wherein the first power transfer device 265 includes a transfer synchronous roller 1654, and a synchronous belt transmission is formed between the first power transfer device 265 and the first manipulation device 30.

For example, the first power transfer device 265 includes a first bearing 1652, a first transmission shaft 1653, a transfer synchronous roller 1654 and a transfer synchronous belt. A hole corresponding to the first power shaft 145 is provided at the bottom of the carrying base 261. A first limiting frame 1651 is provided at a position corresponding to the hole on the carrying base 261, and two first bearings 1652 corresponding to each other are provided at the hole and on the first limiting frame 1651. The first transmission shaft 1653 is connected between the inner rings of the two first bearings 1652, and an end of the first transmission shaft 1653 facing the movable power device 240 is engaged with the first power shaft 145. The first transmission shaft 1653 is also fixedly connected to the two transfer synchronous rollers 1654. The transfer synchronous belt is connected between the transfer synchronous roller 1654 and the first manipulation device 30, so that a synchronous belt transmission is formed between the first power transfer device 265 and the first manipulation device 30.

The structure of the first manipulation device 30 will be described below.

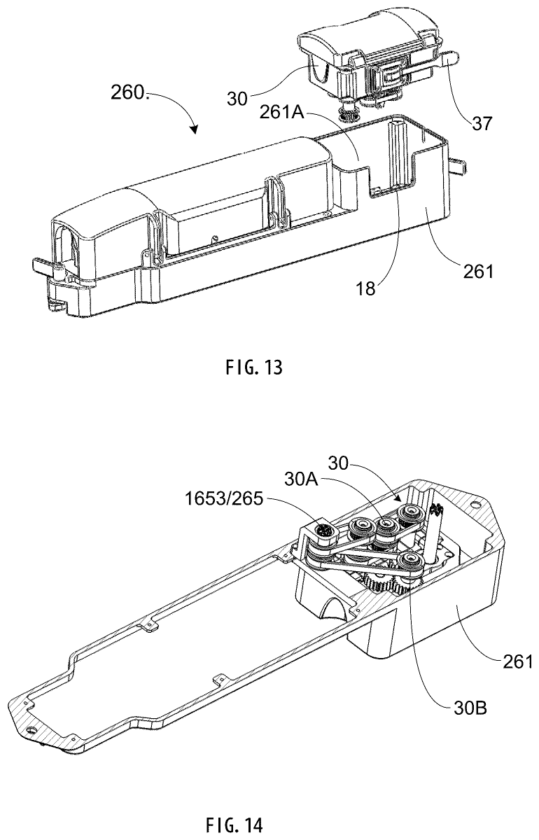

As shown in FIGS. 13 and 14, the first manipulation device 30 is removably disposed in the first part 261A of the carrying base, and it is driven by the first power transfer device 265.

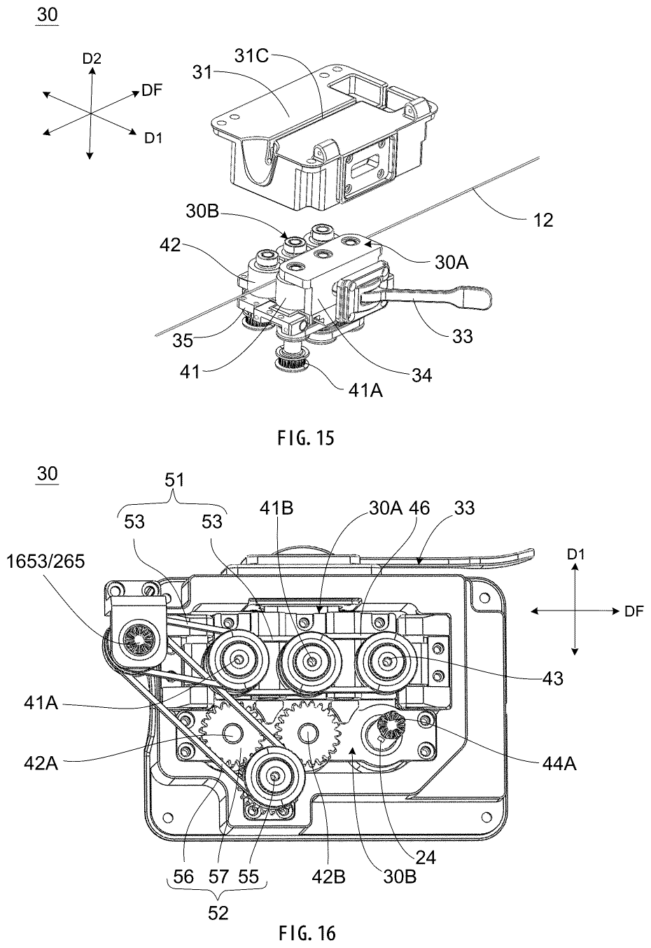

As shown in FIG. 15, the first manipulation device 30 includes a first manipulation device base 31, at least one first roller 41 and at least one second roller 42. Wherein the first manipulation device base 31 is used to be detachably connected to the first carrying device 260 (specifically the first part 261A of the carrying base). The first roller 41 is disposed on the first manipulation device base 31, and an axis of the first roller 41 is not parallel to the conveying direction DF. Optionally, the axis of the first roller 41 is perpendicular to the conveying direction DF, for example, in a second direction D2. The second roller 42 is also disposed on the first manipulation device base 31 and disposed opposite to the first roller 41 (one by one opposite to each other), so as to clamp the guidewire 12 along with the first roller 41. It may be understood that an axis of the second roller 42 is parallel to the axis of the first roller 41. Wherein the first roller 41 and the second roller 42 are both used to be connected to the first transmission device 21 so as to rotate under the driving of the first transmission device 21, and the slave-side execution apparatus 200 is configured such that an rotation direction of the first roller 41 is opposite to an rotation direction of the second roller 42.

It may be understood that when the first roller 41 and the second roller 42 are rotated, the guidewire 12 clamped therebetween is moved along the conveying direction DF under the action of frictional force. Therefore, both the first roller 41 and the second roller 42 are capable of actively rolling under the driving of the driving device, which enables the first manipulation device 30 to have sufficient conveying force. Moreover, since both the first roller 41 and the second roller 42 are capable of rolling actively, even if the structure used to drive the rotation of one of the first roller 41 and the second roller 42 fails, the one may also be passively rotated under the action of frictional force, and it is also possible to achieve that the guidewire 12 is clamped and conveyed jointly by the two sets of rollers.

Optionally, the first manipulation device 30 includes a plurality of first rollers 41 and a plurality of second rollers 42. In the illustrated embodiment, the first manipulation device 30 includes two first rollers 41A, 41B and two second rollers 42A, 42B (see FIG. 16). Optionally, the plurality of first rollers 41 are arranged flush, and/or the plurality of second rollers 42 are also arranged flush. Optionally, the plurality of first rollers 41 are arranged at equal intervals, and/or the plurality of second rollers 42 are also arranged at equal intervals. Optionally, the diameter of the first roller 41 is the same as the diameter of the second roller 42, or the first roller 41 and the second roller 42 are configured as the same roller.

Specifically, as shown in FIGS. 15 to 18, the first manipulation device 30 is divided into two major parts: a first movable roller group 30A and a first fixed roller group 30B. The first movable roller group 30A and the first fixed roller group 30B are arranged along a first direction D1. Wherein the first direction D1 is perpendicular to a plane determined by the conveying direction DF and the axis of the first roller 41. For example, the first direction D1 is perpendicular to a plane determined by the conveying direction DF and the second direction D2 (the conveying direction DF, the first direction D1, and the second direction D2 are perpendicular to each other).

The first roller 41 is included in the first movable roller group 30A, and the second roller 42 is included in the first fixed roller group 30B. The first fixed roller group 30B includes a second fixed seat 35. The second fixed seat 35 is disposed on the first manipulation device base 31 and is immovable relative to the first manipulation device base 31, and the second roller 42 is disposed on the second fixed seat 35. The first movable roller group 30A includes a first movable seat 34. The first movable seat 34 is also disposed on the first manipulation device base 31 and is movable between a first position and a second position relative to the second fixed seat 35 (or the first manipulation device base 31) along the first direction D1. The first roller 41 is disposed on the first movable seat 34. A distance between the first movable seat 34 and the second fixed seat 35 when the first movable seat 34 is in the first position is less than a distance between the first movable seat 34 and the second fixed seat 35 when the first movable seat 34 is in the second position.

Therefore, the distance between the first movable seat 34 and the second fixed seat 35 along the first direction D1 may be changed. That is, the distance between the first movable roller group 30A and the first fixed roller group 30B may be changed. That is, the distance between the first roller 41 and the second roller 42 may be changed. As such, the guidewire 12 may be clamped by the first roller 41 and the second roller 42 when they are close to each other and released when they are far away from each other. As shown in FIGS. 3 and 15, the first manipulation device base 31 is provided with a first base channel 31C for allowing the guidewire 12 to pass through. The first movable roller group 30A and the first fixed roller group 30B are respectively located on both sides of the first base channel 31C, and may clamp the guidewire 12 in the channel 31C.

As shown in FIG. 18, the first manipulation device base 31 is provided with a first guide rod 48 extending along the first direction D1, and the first movable seat 34 is provided with a first guide hole 48A extending along the first direction D1. The first guide rod 48 extends through the first guide hole 48A, so that the first movable seat 34 may be stably moved along the first direction D1 relative to the second fixed seat 35.

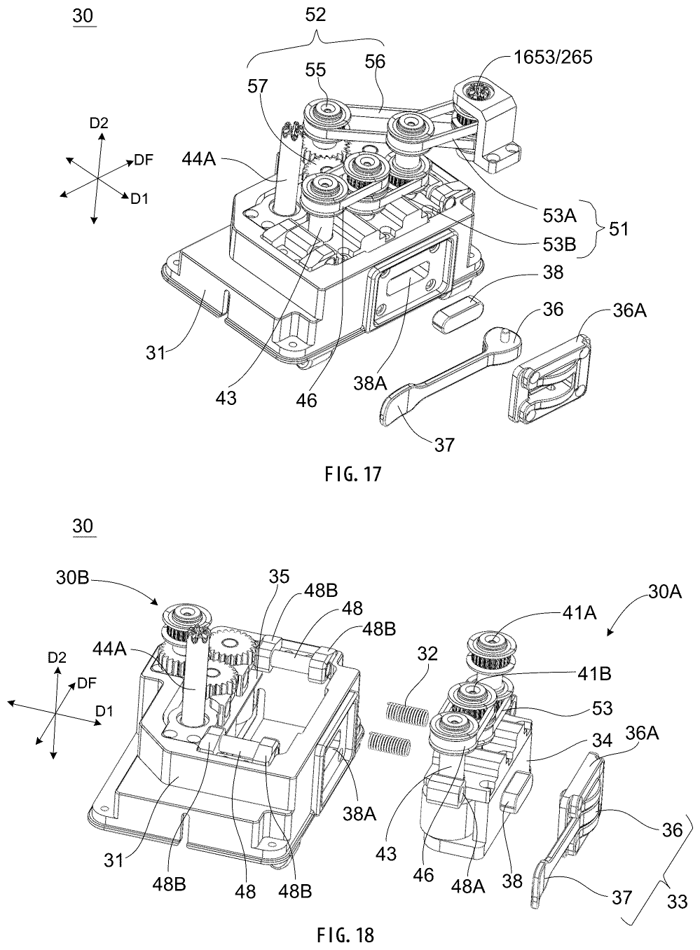

As shown in FIGS. 17 and 18, in order to make the distance between the first roller 41 and the second roller 42 changeable, the first manipulation device 30 further includes a first biasing assembly 32 and a first pressing device 33. The first biasing assembly 32 is connected between the second fixed seat 35 and the first movable seat 34 for providing a force to move the first movable seat 34 away from the second fixed seat 35 along the first direction D1. The first pressing device 33 is disposed on the first manipulation device base 31 and is connected to the first movable seat 34 for operation by the user to bring the first movable seat 34 close to the second fixed seat 35 along the first direction D1.

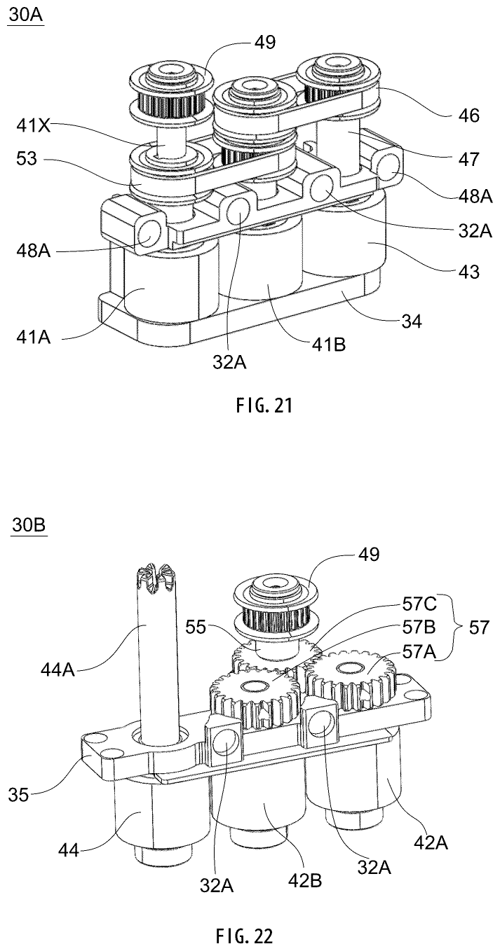

Specifically, the first biasing assembly 32 includes an elastic member that is deformable along the first direction D1. For example, the first biasing assembly 32 is configured as at least one spring 32 extending along the first direction D1. The first movable seat 34 and the second fixed seat 35 are both provided with a first spring blind hole 32A for accommodating both ends of the spring 32 (see FIGS. 21 and 22), so that the spring 32 is stably clamped between the first movable seat 34 and the second fixed seat 35.

The first pressing device 33 includes a cam 36 and a wrench 37. The cam 36 has different radial dimensions. The cam 36 is connected to the first manipulation device base 31 and is rotatable relative to the first manipulation device base 31 about a rotation axis extending along the second direction D2. For example, the cam 36 is mounted to a cam seat 36A, and the cam seat 36A is mounted to the first manipulation device base 31, so that the cam 36 is mounted to the first manipulation device base 31. The cam 36 has different radial dimensions, and its circumferential surface is used to be connected to a side of the first movable seat 34 facing away from the second fixed seat 35. The wrench 37 is connected to the cam 36 for operation by the user to cause the cam 36 to rotate relative to the first manipulation device base 31. It may be understood that in a case where the position of the rotation shaft of the cam 36 is unchanged relative to the first manipulation device base 31, the distance between the first movable seat 34 and the rotation shaft of the cam 36 is changed when different positions of the circumferential surface of the cam 36 contact the first movable seat 34. That is to say, the first movable seat 34 is moved along the first direction D1.

As shown in FIGS. 17 and 18, in order to make it easier for the cam 36 to act on the first movable seat 34, a projection 38 extending along the first direction D1 is connected to a side of the first movable seat 34 facing the cam 36, and is used to contact the circumferential surface of the cam 36. The projection 38 may serve as a part of the first movable seat 34, for example, it may be integrally formed with the first movable seat 34. The projection 38 may also form a separate component from the first movable seat 34. In the illustrated embodiment, the first manipulation device base 31 is provided with a projection groove 38A for accommodating the projection 38. The cam 36 may be at least partially located in the projection groove 38A to contact the projection 38.

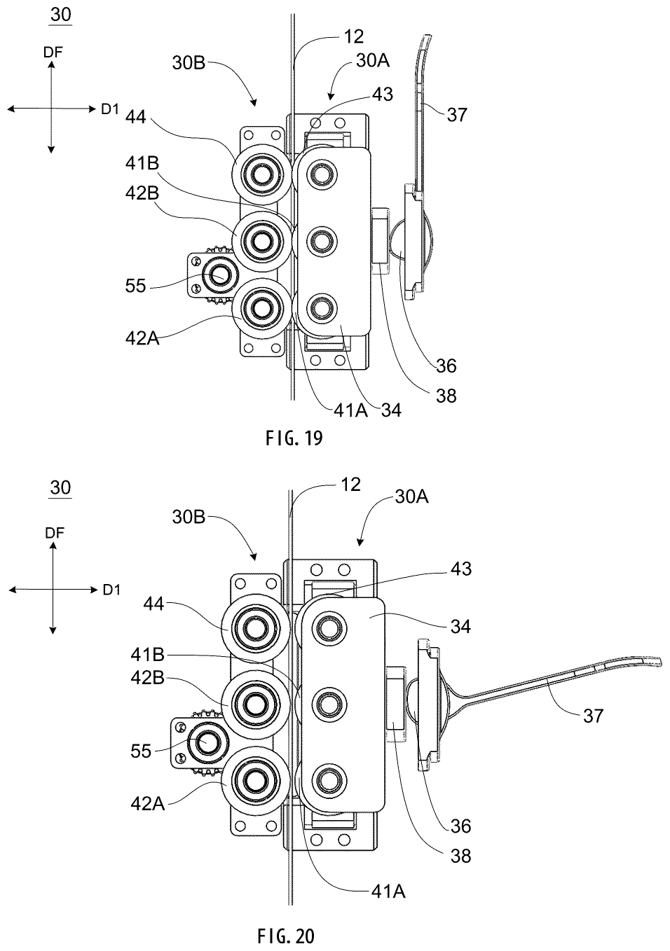

As shown in FIG. 19, when a part of the cam 36 with a larger radius contacts the projection 38, the cam 36 squeezes the first movable seat 34 along the first direction D1 via the projection 38, so that the first movable seat 34 is moved towards the first fixed roller group 30B, and thus the first roller 41 and the second roller 42 are close to each other. At this time, the spring 32 is compressed. As shown in FIG. 20, when a part of the cam 36 with a smaller radius contacts the projection 38, the squeezing force of the cam 36 on the projection 38 is decreased, and then the squeezing force experienced by the spring 32 is decreased. The spring 32 rebounds to push the first movable seat 34 away from the first fixed roller group 30B along the first direction D1 so that the first roller 41 and the second roller 42 are away from each other. The position of the first movable seat 34 in FIG. 19 may be construed as the first position, and the position of the first movable seat 34 in FIG. 20 may be construed as the second position.

As shown in FIG. 17, in order to prevent the first movable roller group 30A from being excessively moved along the first direction D1, optionally, first blocking members 48B are respectively provided at both ends of the first guide rod 48. The first blocking member 48B protrudes in a radial direction of the first guide rod 48, and its radial dimension is larger than the aperture of the first guide hole 48A, thereby preventing the excessive movement of the first guide hole 48A at both ends, that is, the first movable seat 34 will not be excessively moved along the first direction D1. It may be understood that the length of the first guide rod 48 between the two first blocking members 48B will not affect the movement of the first movable seat 34 between the first position and the second position. In other words, the portion of the length of the first guide rod 48 between the two first blocking members 48B which is longer than the length of the first guide hole 48A is not less than the distance between the first position and the second position.

Optionally, the wrench 37 is exposed from the outer surface of the rear consumable box 15, thereby facilitating the user's operation. As shown in FIG. 13, the first carrying device 260 is provided with a window 18 for exposing the wrench 37.

In order to rotate the first roller 41 and the second roller 42, as shown in FIGS. 16 and 17, the first manipulation device 30 further includes a first roller transmission device 51 and a second roller transmission device 52. The first roller transmission device 51 is used to connect the first transmission shaft 1653 and all the first rollers 41 so that the rotation direction of all the first rollers 41 is the same as the rotation direction of the first transmission shaft 1653. The second roller transmission device 52 is used to connect the first transmission shaft 1653 and all the second rollers 42 so that the rotation direction of the second rollers 42 is opposite to the rotation direction of the first transmission shaft 1653. Thus, the rotation directions of the first roller 41 and the second roller 42 are opposite.

Optionally, as shown in FIG. 21, the first roller transmission device 51 includes at least one first manipulation synchronous belt 53, and the first transmission shaft 1653 and the first roller 41 are both connected with a structure (e.g., a synchronous belt clamping ring 49, also referred to as a synchronous roller 49) for connecting the first manipulation synchronous belt 53. The slave-side execution apparatus 200 is configured such that the first transmission shaft 1653 drives all the first rollers 41 to rotate via the first manipulation synchronous belt 53, so that the rotation direction of all the first rollers 41 is the same as the rotation direction of the first transmission shaft 1653. For example, the first roller 41 has a first roller shaft 41X, and the synchronous belt clamping rings 49 are respectively sleeved on the first transmission shaft 1653 and the first roller shaft 41X (the synchronous belt clamping ring 49 sleeved on the first transmission shaft 1653 is also the transfer synchronous roller 1654; since the first transmission shaft 1653 is to drive the first roller 41 and the second roller 42 respectively, the first transmission shaft 1653 is provided with two transfer synchronous rollers 1654). The first manipulation synchronous belt 53 is tightened by two synchronous belt clamping rings 49 so that the first roller shaft 41X is driven to rotate by the first transmission shaft 1653 via the first manipulation synchronous belt 53, causing the first roller 41 to rotate.

It may be understood that the first manipulation synchronous belt 53 is elastic. During the movement of the first movable seat 34 along the first direction D1, the first manipulation synchronous belt 53 may always be tensioned by the two synchronous belt clamping rings 49 of the first transmission shaft 1653 and the first roller shaft 41X due to its elasticity.

For example, the first manipulation device 30 includes N first rollers 41 and N first manipulation synchronous belts 53 (N is an integer greater than or equal to 1). The slave-side execution apparatus 200 may be configured such that each of the first rollers 41 is connected to the first transmission shaft 1653 via one first manipulation synchronous belt 53. That is, each of the first rollers 41 is directly driven by the first transmission shaft 1653. Alternatively, the slave-side execution apparatus 200 may be configured such that each of one part (one or more first rollers 41) of the N first rollers 41 is connected to the first transmission shaft 1653 via one first manipulation synchronous belt 53, and each of the other part of the N first rollers 41 is connected to another first roller 41 via one first manipulation synchronous belt 53. That is, only a part of the first rollers 41 are driven by the first transmission shaft 1653, and the other first rollers 41 are driven to rotate by another first roller 41.

Optionally, the slave-side execution apparatus 200 may be configured such that the first one of the N first rollers 41 is connected to the first transmission shaft 1653 via one first manipulation synchronous belt 53, and each of the other first rollers 41 is connected to the previous first roller 41 via one first manipulation synchronous belt 53. That is, the first one of the first rollers 41 is driven by the first transmission shaft 1653, the second one of the first rollers 41 is driven by the first one of the first rollers 41, the third one of the first rollers 41 is driven by the second one of the first rollers 41, and so on. For example, as shown in FIG. 17, the first manipulation device 30 includes two first rollers 41, i.e., a first roller 41A and a first roller 41B. The first first roller 41A is connected to the first transmission shaft 1653 via one first manipulation synchronous belt 53A, and the second first roller 41B is connected to the first first roller 41 via one first manipulation synchronous belt 53B. In this way, the first manipulation synchronous belts 53 may be evenly distributed. For example, the first manipulation synchronous belts 53 may be made into the same specification, and each of first roller shafts 41X may also have a similar size (each of first roller shafts 41X basically needs to be connected with two synchronous belt clamping rings 49 only), thereby reducing processing difficulty and costs.

The first manipulation synchronous belt 53A is also a transfer synchronous belt in the first power transfer device 265.

With such a connection configuration, in order to detect the slipping of the first manipulation synchronous belt 53, as shown in FIGS. 16 to 22, the first manipulation device 30 is further provided with a first additional roller 43, a second additional roller 44, a first additional roller transmission device 46 and an encoder 24. Wherein the first additional roller 43 is disposed on the first movable seat 34, for example, with the N first rollers 41 being disposed side by side on the first movable seat 34. The second additional roller 44 is disposed on the second fixed seat 35, for example, with the N second rollers 42 being disposed side by side on the second fixed seat 35. The second additional roller 44 is disposed opposite to the first additional roller 43, so as to be able to clamp the guidewire 12 along with the first additional roller 43. The first additional roller transmission device 46 is used to connect the first additional roller 43 with the Nth one of the first rollers 41, so that the first additional roller 43 is rotated in the same direction as the rotation direction of the first transmission shaft 1653 under the driving of the Nth one of the first rollers 41. The encoder 24 is connected to the second additional roller 44 for detecting the rotation angle of the second additional roller 44. The encoder 24 is electrically connected to the control module so that the rotation angle of the second additional roller 44 may be obtained by the control module.

In this way, in a case where the first manipulation synchronous belt 53 does not slip, all the first rollers 41 are rotated normally. Under the action of the first additional roller transmission device 46, the first additional roller 43 rotates actively. The guidewire 12 is clamped by the first additional roller 43 along with the second additional roller 44 so that the second additional roller 44 is passively rotated under the action of frictional force, and thereby the rotation angle detected by the encoder 24 will change continuously. Since the first transmission shaft 1653 forms a one-by-one driving connection relationship with the N first rollers 41, if any one of the first manipulation synchronous belts 53 slips or breaks, the Nth one of the first rollers 41 (the last one of the first rollers 41) cannot be rotated, so that the first additional roller 43 and the second additional roller 44 cannot be rotated, and the rotation angle recorded by the encoder 24 will not change. The failure of the apparatus can thus be detected by the control module.

Optionally, as shown in FIGS. 16 to 18, the encoder 24 is connected to an axle 44A of the second additional roller 44, and is configured to detect the rotation of the second additional roller 44 by detecting the rotation of the second additional roller axle 44A.

As shown in FIGS. 5 and 11, the movable power device 240 further includes a third rotating shaft 293 for connecting to the second additional roller axle 44A and rotating synchronously with the second additional roller axle 44A. Wherein the encoder 24 is provided to the third rotating shaft 293. The third rotating shaft 293 is, for example, disposed on the base 142 and penetrates through the base 142, and an end of the third rotating shaft 253 facing the first carrying device 260 is in transmission connection with the second additional roller axle 44A, e.g., by a gear engagement connection.

Optionally, the first additional roller transmission device 46 is configured as a synchronous belt. For example, in the illustrated embodiment, the second one of the first rollers 41 is connected to the axle 47 of the first additional roller 43 via the second manipulation synchronous belt 46 to drive the first additional roller 43 to rotate.

Optionally, the diameter of the first additional roller 43 is the same as that of the first roller 41, and the diameter of the second additional roller 44 is the same as that of the second roller 42. Optionally, the diameter of the first additional roller 43 is the same as that of the second additional roller 44. In other words, the first roller 41, the second roller 42, the first additional roller 43 and the second additional roller 44 are configured as the same roller. Optionally, an axis of the first additional roller 43 is parallel to the axis of the first roller 41, and an axis of the second additional roller 44 is parallel to the axis of the second roller 42. In other words, the first roller 41, the second roller 42, the first additional roller 43 and the second additional roller 44 are configured to be arranged parallel to each other.

As shown in FIGS. 16 and 17, the second roller transmission device 52 includes a fourth transmission shaft 55, a first transmission device 56 for the second roller, and a second transmission device 57 for the second roller. The fourth transmission shaft 55 is disposed on the first manipulation device base 31. The first transmission device 56 for the second roller is used to connect the first transmission shaft 1653 and the fourth transmission shaft 55 so that the fourth transmission shaft 55 is rotated under the driving of the first transmission shaft 1653. The second transmission device 57 for the second roller is used to connect the fourth transmission shaft 55 and all the second rollers 42 so that all the second rollers 42 are rotated under the driving of the fourth transmission shaft 55.

Optionally, one of the first transmission device 56 for the second roller and the second transmission device 57 for the second roller is configured to be driven by a synchronous belt, and the other thereof is configured to be driven by a gear. It may be understood that the synchronous belt transmission mode does not change the rotation direction (the rotation direction of the driven member is the same as that of the driving member), while the gear transmission mode may change the rotation direction. Thus, with such a configuration, the rotation direction of all the second rollers 42 may be opposite to that of the first transmission shaft 1653.

Optionally, the first manipulation device 30 is configured such that the component of the first roller transmission device 51 for connecting the first transmission shaft 1653 and the component of the second roller transmission device 52 for connecting the first transmission shaft 1653 are both synchronous belts. That is, the component of the first roller transmission device 51 for connecting the first transmission shaft 1653 and the first roller 41 is a synchronous belt (the first manipulation synchronous belt 53), and the first transmission device 56 for the second roller is also configured as a synchronous belt (the third synchronous belt 56). That is, the first transmission shaft 1653 is connected to the fourth transmission shaft 55 through the third synchronous belt 56. In this way, the first roller 41 and the second roller 42 are driven by the first transmission shaft 1653 with the same driving mode, which is conducive to simplifying the design. The third synchronous belt 56 is also the transfer synchronous belt in the first power transfer device 265.

Therefore, as shown in FIG. 22, the second transmission device 57 for the second roller is configured as a gear assembly. In the illustrated embodiment, the first manipulation device 30 includes two second rollers 42, i.e., a second roller 42A and a second roller 42B. The gear assembly 57 includes a gear 57C coaxially connected to the fourth transmission shaft 55, a gear 57A coaxially connected to the second roller 42A and a gear 57B coaxially connected to the second roller 42B. The gear 57C is engaged with the gear 57A and gear 57B respectively. Thus, the rotation direction of the fourth transmission shaft 55 is opposite to the rotation direction of the first transmission shaft 1653, and the rotation direction of the second roller 42 is opposite to the rotation direction of the fourth transmission shaft 55. That is, the rotation direction of the second roller 42 is opposite to the rotation direction of the first transmission shaft 1653.

In the present application, the first additional roller 43 and the second additional roller 44 also participate in the conveying of the guidewire 12, and may also be considered as a part of the first manipulation device 30. The first additional roller 43 may also be considered as one first roller 41. The second additional roller 44 may also be considered as one second roller 42 that does not have self-driving force and cannot rotate actively.

It is to be understood that in the above-mentioned device of transmission via the synchronous belt, the transmission may be completed by arranging the synchronous belt clamping rings 49 on the driving shaft and the driven shaft so that the synchronous belt is tightened by the two synchronous belt clamping rings 49. During operation, the shaft and the synchronous belt clamping rings 49 are connected, for example, by a key and a keyway, so that they are synchronously rotatable, and the synchronous belt clamping rings 49 and the synchronous belt are rotated synchronously by frictional force.

In the present application, the first manipulation device 30 may also be referred to as a first conveying assembly 30, and the first driving device 143 may be referred to as a first power assembly 143.

In actual application, the base 142 of the movable power device 240 extends substantially along a vertical plane, and the first power shaft 145 and the first transmission shaft 1653 extend substantially along a horizontal direction. This allows the first carrying device 260 to be connected to the side of the movable power device 240, and the first roller 41 and the second roller 42 of the first manipulation device 30 both extend substantially along the horizontal direction, that is, the first roller 41 and the second roller 42 are opposite to each other along a vertical direction.

In the present application, the specific structure of the first transmission device 21 is not limited. It may be understood that the first transmission device 21 includes a portion disposed in the movable power device 240 and a portion disposed in the first carrying device 260.

The following describes how the second manipulation device 290 is controlled by the slave-side execution apparatus 200.

The slave-side execution apparatus 200 further includes a second transmission device 22 connected between the second driving device and the second manipulation device 290 and configured to transfer the driving force of the second driving device to the second manipulation device 290.

The second transmission device 22 is similar to the first transmission device 21, and includes a portion disposed in the movable power device 240 and a portion disposed in the first carrying device 260. As shown in FIGS. 5, 11 and 12, the second transmission device 22 includes a second power shaft 146, a second transmission assembly 95 and a second power transfer device 266.

The second power shaft 146 is used to connect the second manipulation device 290 and is disposed on the movable power device 240. The second power shaft 146 is configured to be rotatable about its own axis relative to the movable power device 240. The second power shaft 146 is, for example, disposed on the base 142 and penetrates through the base 142, and an end of the second power shaft 146 facing the first carrying device 260 is in transmission connection with the second manipulation device 290.

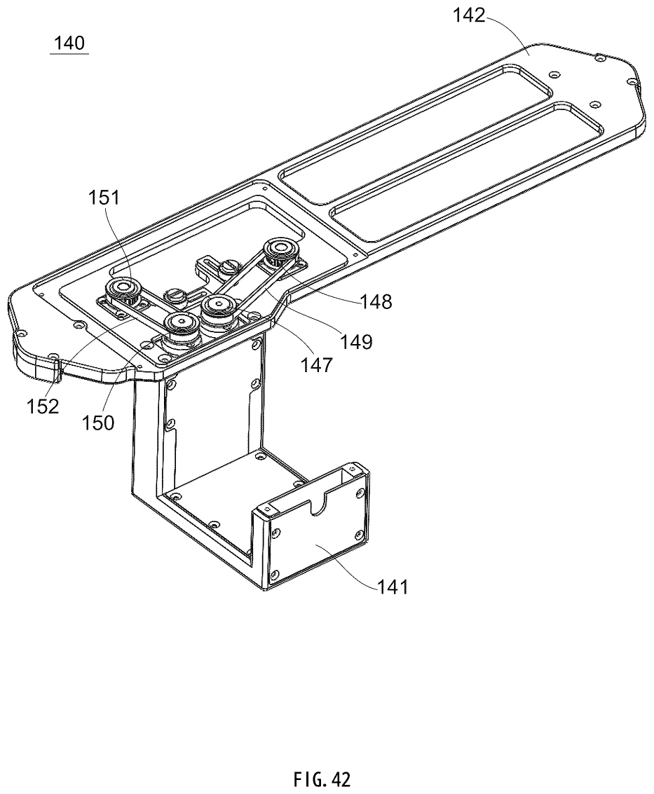

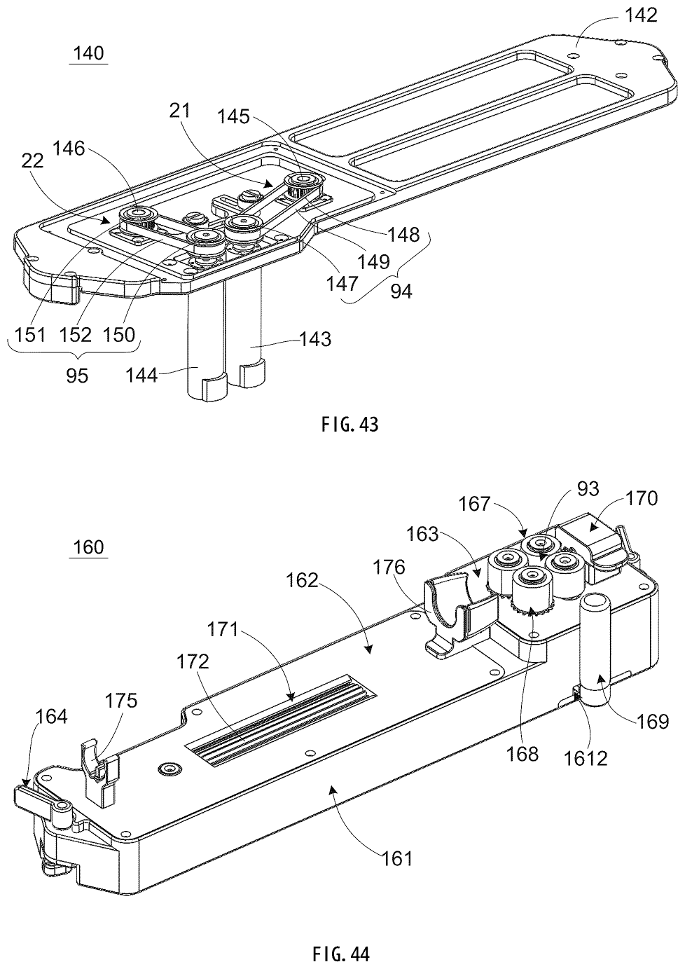

The second driving device 144 is, for example, disposed on the connecting frame 141 of the movable power device 240. The second transmission assembly 95 is disposed on the movable power device 240, e.g., on the base 142. The second transmission assembly 95 is connected between the second driving device 144 and an end of the second power shaft 146 facing away from the first carrying device 260, so that the second power shaft 146 is rotated under the driving of the second driving device 144. For example, the second transmission assembly 95 includes a second driving roller 150 (referring to FIGS. 42 and 43), a second driven roller 151 and a second synchronous belt 152. The second driving roller 150 is connected to a driving end of the second driving device 144. The second driven roller 151 is connected to an end of the second power shaft 146 facing away from the first carrying device 260. The second synchronous belt 152 is connected between the second driving roller 150 and the second driven roller 151 to transfer the power generated by the second driving device 144 to the second power shaft 146 so as to cause it to rotate.

The second power transfer device 266 is disposed in the first carrying device 260, e.g., in the second part 261B of the carrying base. The second power transfer device 266 is connected between the second manipulation device 290 and the second power shaft 146 so that the second power shaft 146 drives the second manipulation device 290 to operate. The position of the second power transfer device 266 corresponds to the position of the second power shaft 146, and the second power transfer device 266 is engaged with the second power shaft 146. Wherein the second power transfer device 266 includes a transfer gear 1664, and a gear engaging transmission is formed between the second power transfer device 266 and the second manipulation device 290.

For example, the second power transfer device 266 includes a second limiting frame 1661, a second bearing 1663, a second transmission shaft 1662 and the transfer gear 1664. A hole corresponding to the second power shaft 146 is provided at the bottom of the carrying base 261. The second limiting frame 1661 is disposed on the carrying base 261 and corresponds to the hole, and the second bearing 1663 is provided on the second limiting frame 1661. The second transmission shaft 1662 is connected to the inner ring of the second bearing 1663, and an end of the second transmission shaft 1662 facing the movable power device 240 is engaged with the second power shaft 146. An end of the second transmission shaft 1662 facing away from the movable power device 240 is fixedly connected with the transfer gear 1664. The transfer gear 1664 is configured to connect the second manipulation device 290 so that a gear engaging transmission is formed between the second power transfer device 266 and the second manipulation device 290. The transfer gear 1664 is configured, for example, as a bevel gear.

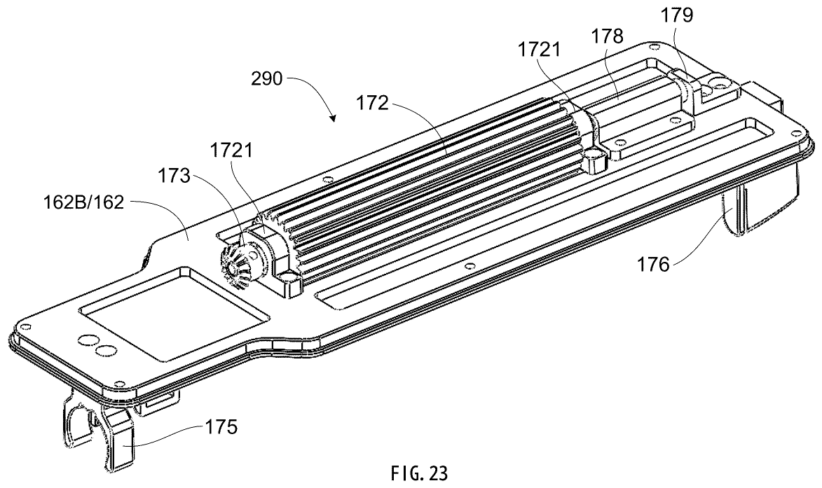

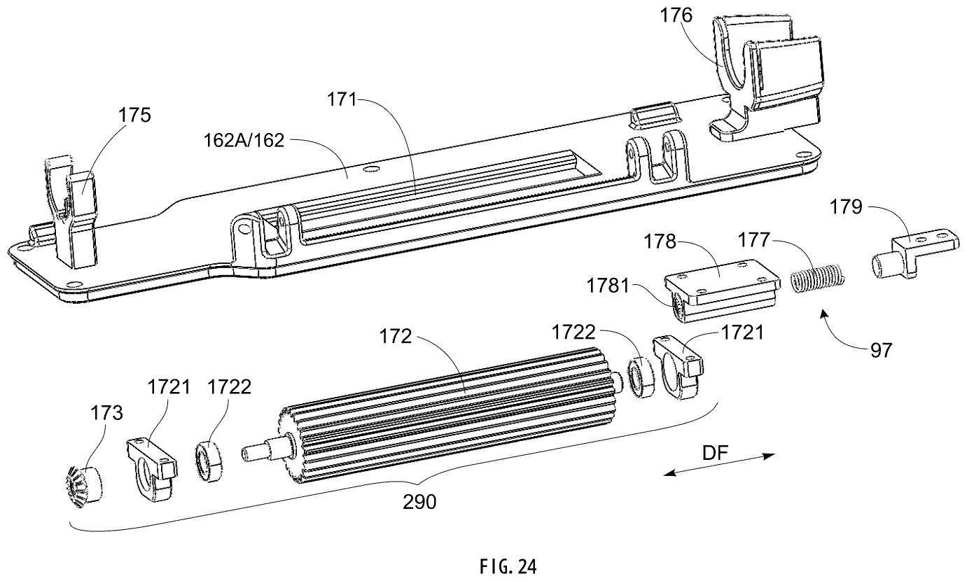

As shown in FIGS. 23 and 24, as described above, the carrying base 261 is provided with the functional manipulation seat 162 for mounting the medical apparatus 10. The functional manipulation seat 162 includes a first side 162A of the functional manipulation seat and a second side 162B of the functional manipulation seat arranged opposite to each other. Wherein the medical apparatus 10 is detachably connected to the first side 162A of the functional manipulation seat. The functional manipulation seat 162 is provided with an operation opening 171.

The second manipulation device 290 includes a functional gear 172 and a bevel gear 173. The functional gear 172 is rotatably disposed on the second side 162B of the functional manipulation seat, and at least partially exposed from the operation opening 171 to the first side 162A of the functional manipulation seat, so as to be able to form an engagement with the operating gear 13 of the medical apparatus 10. The bevel gear 173 is coaxially connected to the functional gear 172, and thus synchronously rotatable with the functional gear 172. The bevel gear 173 is engaged with the transfer gear 1664 of the second power transfer device 266. When the transfer gear 1664 is rotated, the transfer gear 1664 drives the bevel gear 173 to rotate, which in turn drives the functional gear 172 to rotate and finally drives the operating gear 13 to rotate.

Specifically, the second side 162B of the functional manipulation seat is provided with two gear seats 1721 at both ends of the operation opening 171. One third bearing 1722 is provided on each of the two gear seats 1721, and two ends of the rotating shaft of the functional gear 172 are fixedly connected to the inner rings of two third bearings 1722, respectively. One end of the rotating shaft of the functional gear 172 is also fixedly connected to the bevel gear 173 so that the functional gear 172 and the bevel gear 173 are coaxially rotatable. The second side 162B of the functional manipulation seat is used to face the inside of the second part 261B of the carrying base, and the transmission device 266 is disposed in the second part 261B of the carrying base, so that it is easy for the bevel gear 173 to engage with the transfer gear 1664.

The first carrying device 260 also includes a supporting structure 97 for supporting the medical apparatus 10. The supporting structure 97 is disposed on the functional manipulation seat 162, and includes a first bracket 175, a second bracket 176 and a second elastic member 177. The first bracket 175 is disposed on the first side 162A of the functional manipulation seat. The second bracket 176 is also disposed on the first side 162A of the functional manipulation seat and is spaced apart from the first bracket 175 along the conveying direction DF to support the medical apparatus 10 along with the first bracket 175. The second bracket 176 is configured to be movable relative to the functional manipulation seat 162 along the conveying direction DF. The second elastic member 177 is connected to the second bracket 176 for applying a force to the second bracket 176 to move the second bracket 176 away from the first bracket 175 along the conveying direction DF.

Specifically, the functional manipulation seat 162 is provided with an opening (not shown) extending along the conveying direction DF, and a portion of the second bracket 176 is disposed in the opening and is movable in the opening along the conveying direction DF. The supporting structure 97 further includes a connecting seat 178 and a bottom bracket 179. The connecting seat 178 is disposed at the second side 162B of the functional manipulation seat, and is closer to the first bracket 175 relative to the opening. The connecting seat 178 has a channel 1781 extending along the conveying direction DF. The bottom bracket 179 is connected to the portion of the second bracket 176 located in the opening, and at least partially extends into the channel 1781. Wherein the second elastic member 177 is disposed in the channel 1781 and connected between the connecting seat 178 and the bottom bracket 179. The second elastic member 177 is, for example, a spring. Thus, the first bracket 175 and the second bracket 176 may be used for mounting the medical apparatus 10, and the medical apparatus 10 may be conveniently clamped and removed by using the movable second bracket 176.

In an embodiment not shown in the present application, the first carrying device 260 is provided with a plurality of second manipulation devices 290 for respectively operating different medical apparatuses 10 to achieve different medical functions. Alternatively, the plurality of second manipulation devices 290 are respectively used to operate different medical function mechanisms 13 of the medical apparatus 10.

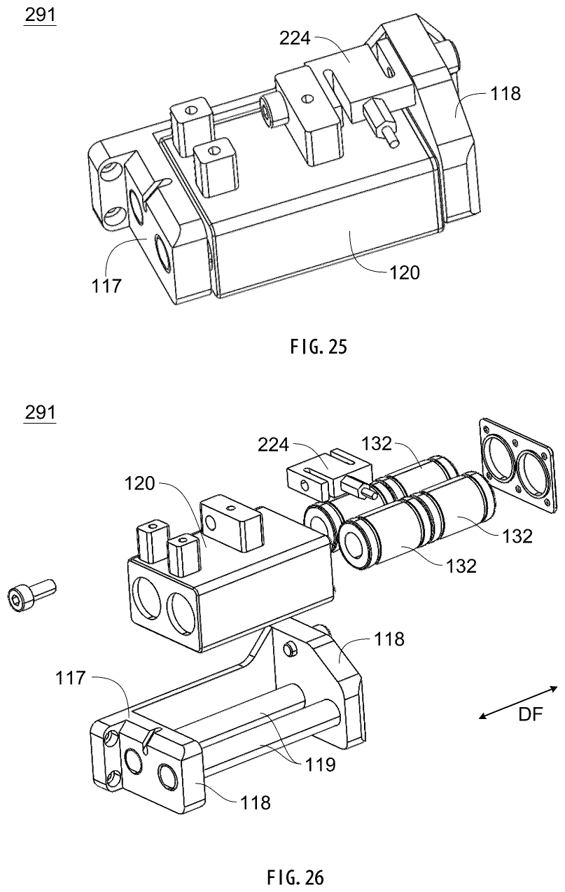

FIGS. 25 and 26 show another example of a movable assembly 291 in which the movable frame 117 has two second guide rails 119 arranged vertically at intervals and extending parallel to each other. In addition, the movable assembly 291 is also driven to move by a drive structure other than the lead screw pair. For example, the movable frame 117 is provided with a rack extending along the conveying direction DF. The main body power device 210 includes a gear that engages with the rack. The main body driving device 124 drives the gear to rotate, the gear transfers power to the rack, and the rack drives the movable frame 117 to move.

The structure of the second conveying assembly 60 is described below.

As shown in FIG. 2, the second conveying assembly 60 includes a second conveying assembly base 61, at least one third roller and at least one fourth roller. Wherein the second conveying assembly base 61 is detachably connected to the second power assembly 14. The third roller is disposed on the second conveying assembly base. An axis of the third roller is not parallel to the conveying direction DF. Optionally, the axis of the third roller is perpendicular to the conveying direction DF, e.g., is a second additional direction D21. The fourth roller is disposed on the second conveying assembly base 61, and is arranged opposite to the third roller (one by one opposite to each other), so as to clamp the guidewire 12 and/or the catheter 11 along with the third roller. It may be understood that the axis of the fourth roller is parallel to the axis of the third roller.

In the second conveying assembly 60, the third roller may be a driving roller (driven to rotate by the fourth driving device), and the fourth roller may be a driven roller.

According to the aforementioned method of using the slave-side execution apparatus 200 in surgery, even if the fourth driving device fails, the third roller and the fourth roller may be passively rotated under the action of frictional force to achieve the clamping and supporting of the guidewire 12 and/or the catheter 11. Accordingly, in the present application, the first roller 41 and the second roller 42 are designed as driving rollers, which may ensure the stable operating performance of the slave-side execution apparatus 200 to a great extent. Of course, it is also possible to control whether the fourth driving device outputs the driving force according to needs of the surgery, i.e., to flexibly control the third roller as a driving roller or a driven roller.

Optionally, the diameter of the third roller is the same as that of the fourth roller. In other words, the third roller is configured as the same roller as the fourth roller.

The second conveying assembly base 61 is connected to the second power assembly 14 by, for example, locking, magnetic attraction, etc.

In the embodiments shown in FIGS. 1 and 2, the axial direction D21 of the fourth roller and the third roller is an up-down direction. The fourth and third rollers are spaced apart along a horizontal first additional direction D11. Optionally, the distance between the fourth roller and the third roller is adjustable to accommodate the guidewires 12 and/or the catheters 11 of different diameters.

FIGS. 28 to 34 show the second power assembly 14 and the second conveying assembly 60 of another example.

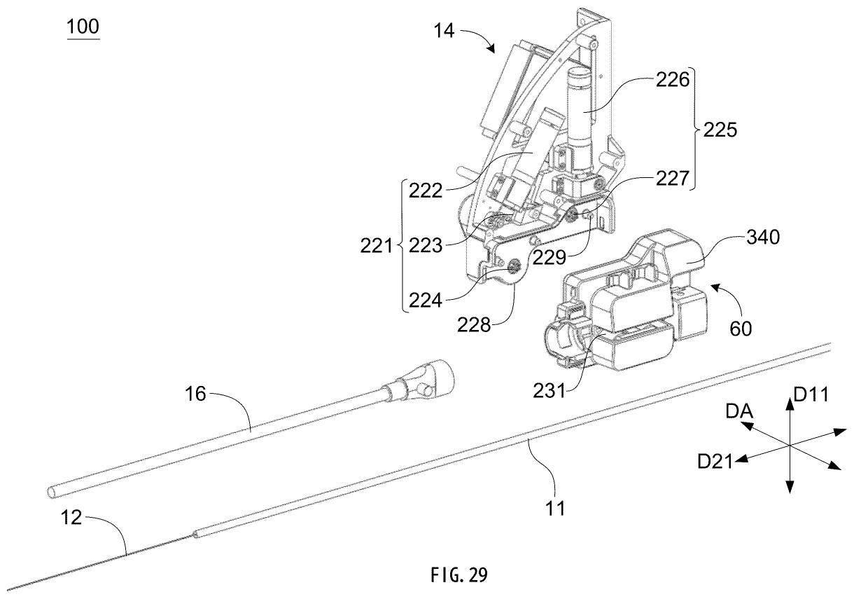

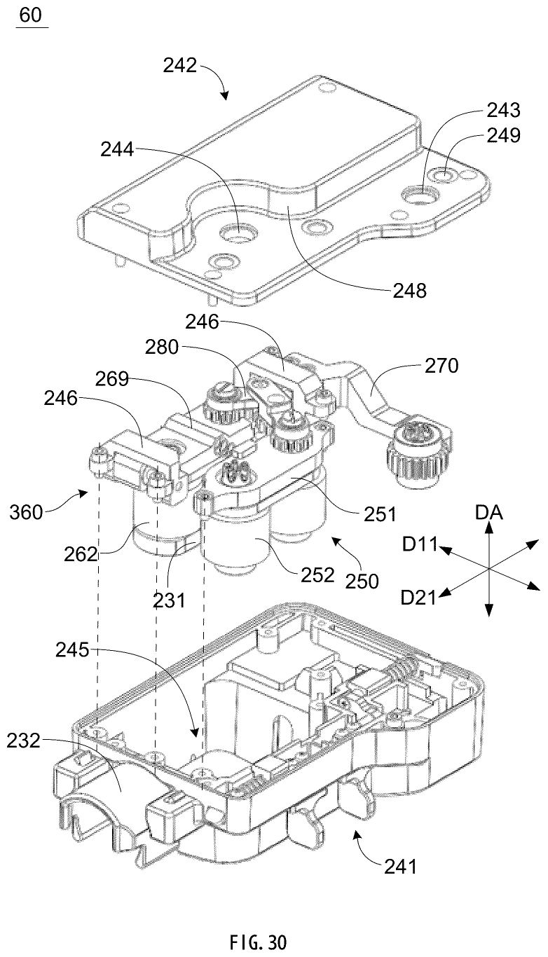

As shown in FIGS. 29 and 30, the second conveying assembly 60 includes at least one pair of third rollers 252 and fourth rollers 262 arranged oppositely, and the axes of the third rollers 252 and the axes of the fourth rollers 262 are parallel and are spaced apart along the first additional direction D11 so that a channel 231 extending along the second additional direction D21 is formed therebetween. Preferably, the axis of the third roller 252 (extending along an axial direction DA), the first additional direction D11, and the second additional direction D21 are perpendicular to each other. The guidewire 12 and/or the catheter 11 pass through the channel 231, and the third roller 252 and the fourth roller 262 arranged opposite each other are used to jointly clamp the guidewire 12 and/or the catheter 11. The third roller 252 is rotatable about its own axis under the driving of the fourth driving device 221 of the second power assembly 14. Even if only one third roller 252 is rotated about its own axis under the driving of the fourth driving device 221 of the second power assembly 14, the second conveying assembly 60 may cause all the third rollers 252 and all the fourth rollers 262 to rotate by frictional force. Thus, the guidewire 12 and/or the catheter 11 are clamped and conveyed jointly by the third roller 252 and the fourth roller 262 arranged opposite each other.

In the present application, a driving device refers to a device that includes an active component that may output a driving force, such as a component that operates with a power source and outputs torque, for example an electric control component such as a motor, cylinder, etc. The active component, also known as an active drive component, usually operates under the control of the control module (e.g., a processor module 302). The control module controls the operation of the drive components according to the signal monitored by the sensor or the operating parameters of the drive components, so that the device may operate in a controlled manner.

When the second conveying assembly 60 includes a plurality of pairs of third rollers 252 and fourth rollers 262 arranged opposite each other along the first additional direction D11, the plurality of pairs of the third rollers 252 and the fourth rollers 262 are arranged in parallel along the second additional direction D21. Alternatively, a plurality of third rollers 252 are arranged in parallel along the second additional direction D21, and a plurality of fourth rollers 262 are arranged in parallel along the second additional direction D21. The third rollers 252 and the fourth rollers 262 are arranged in one-to-one correspondence. Preferably, the third roller 252 and the fourth roller 262 are configured as the same rollers (at least the same diameter). In the present application, the direction in which the guidewire 12 and/or the catheter 11 enter the blood vessel is forward, and the direction in which the guidewire 12 and/or the catheter 11 exit the blood vessel is rear. It may also be understood that the second additional direction D21 is a bidirectional direction, where one direction is the forward and the other direction is the rear. The guidewire 12 and/or the catheter 11 is allowed to be conveyed more stably by providing a plurality of pairs of third rollers 252 and fourth rollers 262.

The second conveying assembly 60 may also be used to mount a large sheath 16 through which the catheter 11 extends. The guidewire 12 extends through the catheter 11.

Specifically, as shown in FIGS. 29 and 30, the second conveying assembly 60 includes a box 340, a third roller assembly 250, and a fourth roller assembly 360. The third roller assembly 250 includes the third roller 252. The fourth roller assembly 360 includes the fourth roller 262. The third roller assembly 250 and the fourth roller assembly 360 are both disposed in the box 340. The box 340 is detachably connected to the second power assembly 14.

The box 340 constitutes the housing of the second conveying assembly 60, including, for example, a box body 241 and a box cover 242. The box body 241 has an accommodation space 245 for accommodating respective components of the second conveying assembly 60. The box cover 242 is, for example, connected to the box body 241 by bolts, thereby enclosing the accommodation space 245 to a certain extent.

The second power assembly 14 may be detachably connected to the box cover 242. For example, the second power assembly 14 is provided with a pin 229, and the box cover 242 is provided with a mounting hole 249 to accommodate the pin 229. Thus, the second conveying assembly 60 may be mounted to the second power assembly 14 (the second conveying assembly 60 is hung on the side wall of the second power assembly 14) by holding the box 340 with hands and fitting the mounting hole 249 over the pin 229 so that the pin 229 is inserted into the mounting hole 249, and the second conveying assembly 60 may be detached from the second power assembly 14 by the reverse operation of removing the pin 229 from the mounting hole 249. The end of the pin 229 and/or the wall of the mounting hole 249 may be provided with a guide surface so that the pin 229 may be easily inserted into the mounting hole 249. The second power assembly 14 may also be provided with a second power assembly limiting surface 228, and the box cover 242 may also be provided with a second conveying assembly limiting surface 248. The second power assembly limiting surface 228 matches the shape of the second conveying assembly limiting surface 248 to guide the mounting of the second conveying assembly 60 through mutual contact and to position the second conveying assembly 60 relative to the second power assembly 14. The second power assembly limiting surface 228 and the second conveying assembly limiting surface 248 include, for example, a feature shape (e.g., an arc) by which they are positioned relative to each other.

The box body 241 is equivalent to the second conveying assembly base, also referred to as a second conveying assembly base 241. The third roller assembly 250 and the fourth roller assembly 360 are both disposed on the second conveying assembly base 241. A large sheath mounting portion 232 for mounting the large sheath 16 is also provided on the second conveying assembly base 241.

The third roller assembly 250 includes a fifth fixed seat 251 located in the accommodation space 245 and disposed at the second conveying assembly base 241. The position of the fifth fixed seat 251 relative to the second conveying assembly base 241 is maintained unchanged, for example they are connected by bolts. The third roller 252 is disposed on the fifth fixed seat 251. The fourth roller assembly 360 includes a fifth movable seat 269 located in the accommodation space 245 and disposed at the second conveying assembly base 241. The fourth roller 262 is disposed on the fifth movable seat 269. The fifth fixed seat 251 and the fifth movable seat 269 are arranged along the first additional direction D11.

The third roller assembly 250 may include a plurality of third rollers 252. The third roller assembly 250 is configured such that one of the third rollers 252 is used for rotating about its own axis under the driving of the fourth driving device 221, and each of the other third rollers 252 is rotated about its own axis under the driving of another third roller 252. Thus, the fourth driving device 221 only needs to be connected with one third roller 252, the connection between the fourth driving device 221 and the second conveying assembly 60 is simple.

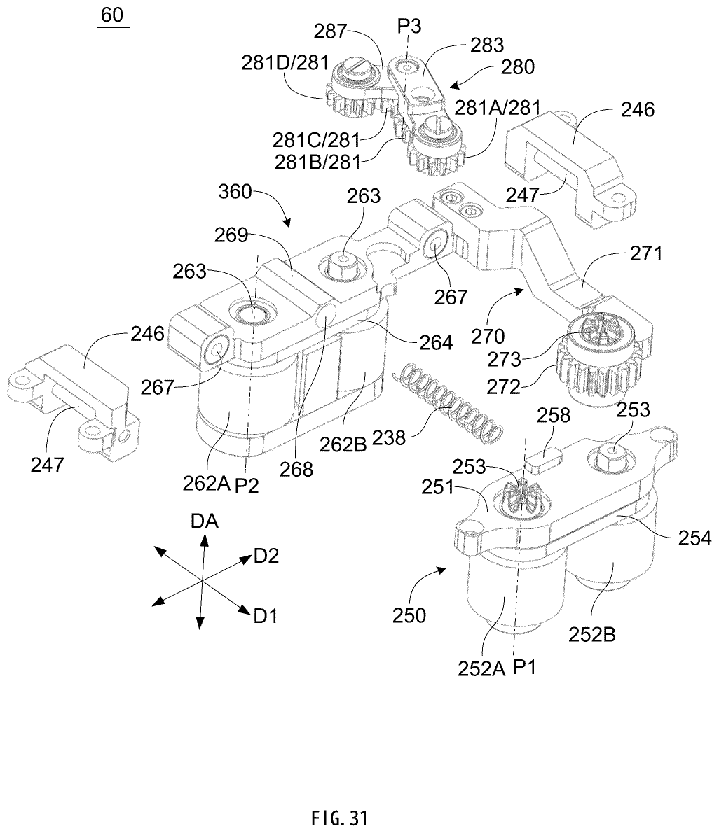

For example, as shown in FIG. 31, the third roller assembly 250 may further include a plurality of first rotating shafts 253 and at least one first transmission belt 254. The first rotating shaft 253 is correspondingly arranged with the third roller 252 and is coaxially connected with the corresponding third roller 252. That is, each third roller 252 has its own first rotating shaft 253, and the two share a first axis P1. The third roller 252 may, for example, be sleeved on the periphery of the first rotating shaft 253, and they are connected by the key and the keyway so that they are synchronously rotatable. The third roller 252 may also be integrally formed with the first rotating shaft 253. The first rotating shaft 253 may be connected in a shaft hole of the fifth fixed seat 251 via a bearing, so that the third roller 252 is connected to the fifth fixed seat 251 through the first rotating shaft 253 and then connected to the second conveying assembly base 241 through the fifth fixed seat 251. Wherein a part of the first rotating shafts 253 are used for rotating about its own axis Pl under the driving of the fourth driving device 221, each first transmission belt 254 is connected to two first rotating shafts 253, and the other rotating shafts are all rotated under the driving of the first transmission belt 254. For example, a clamping ring is disposed around the periphery of the first rotating shaft 253, and the first transmission belt 254 is tensioned by the clamping rings of two first rotating shafts 253, so that the two first rotating shafts 253 are connected through the first transmission belt 254 to rotate synchronously. Thus, a part of the third rollers 252 are rotated under the driving of the fourth driving device 221, and the remaining third rollers 252 are each driven to rotate by another third roller 252 via the first transmission belt 254.

In the illustrated embodiment, one first rotating shaft 253 is used for rotating about its own axis Pl under the driving of the fourth driving device 221. The number of the first transmission belts 254 is one less than the number of the third rollers 252, that is, one less than the number of the first rotating shafts 253. Thus, one third roller 252 is rotated under the driving of the fourth driving device 221, and the remaining third rollers 252 are each driven to rotate by another third roller 252 through the first transmission belt 254.

Specifically, as shown in FIG. 29, the fourth driving device 221 includes a fourth driving component 222, a fourth transmission device 223 and a fourth driving shaft 224. The fourth driving component 222 is, for example, configured as a motor, e.g., a fourth servomotor. The fourth transmission device 223 may be configured as a gear assembly, which is connected between an output shaft of the fourth servomotor and the fourth driving shaft 224, so that the fourth driving shaft 224 may be driven to rotate by the fourth servomotor. When the second conveying assembly 60 is mounted to the second power assembly 14, the fourth driving shaft 224 is coaxially connected with one first rotating shaft 253 so that the one first rotating shaft 253 may be rotated synchronously with the fourth driving shaft 224. The fourth driving shaft 224 and the one first rotating shaft 253 may form a snap-fit connection with each other by the spline at the end.

As shown in FIG. 30, the box cover 242 is provided with a first through hole 244 through which the fourth driving shaft 224 passes.

In the illustrated embodiment, the second conveying assembly 60 includes two third rollers 252 (i.e., the second conveying assembly 60 includes two first rotating shafts 253) and one first transmission belt 254. The two third rollers 252 are the first third roller 252A and the second third roller 252B, respectively. The fourth driving shaft 224 is connected to the first rotating shaft 253 of the first third roller 252A. The two first rotating shafts 253 are connected via the first transmission belt 254.

The fifth movable seat 269 is configured to be movable relative to the second conveying assembly base 241 along the first additional direction D11 so that the fifth movable seat 269 is movable relative to the fifth fixed seat 251 along the first additional direction D11, and the fourth roller 262 is movable relative to the third roller 252 along the first additional direction D11. The distance between the fourth roller 262 and the third roller 252 is changeable, that is, the width of the channel 231 is changeable, so that the second conveying assembly 60 may clamp the guidewires 12 and/or the catheters 11 of different diameters.

For example, the second conveying assembly 60 further includes a guide member 246 located in the accommodation space 245 and disposed at the second conveying assembly base 241, e.g., mounted to the second conveying assembly base 241 by bolts. The guide member 246 extends along the first additional direction D11. The fifth movable seat 269 is connected to the guide member 246 and is movable relative to the guide member 246 along the first additional direction D11. For example, the guide member 246 includes a guide rod 247 extending along the first additional direction D11. The fifth movable seat 269 includes a guide rod through hole 267. The guide rod 247 extends through the guide rod through hole 267, and is movable in the guide rod through hole 267 along the first additional direction D11 relative to the guide rod through hole 267. When the position of the guide member 246 is unchanged relative to the second conveying assembly base 241, the fifth movable seat 269 is connected to the second conveying assembly base 241 via the guide member 246 and is movable relative to the second conveying assembly base 241.

In the illustrated embodiment, in order to allow the fifth movable seat 269 to move stably, one guide rod through holes 267 is provided at each of the opposite ends of the fifth movable seat 269 along the second additional direction D21, and two guide members 246 are correspondingly disposed in the accommodation space 245.

To enable the fifth movable seat 269 to move, as shown in FIG. 30 to FIG. 33, the second conveying assembly 60 further includes a position adjustment assembly 270. The position adjustment assembly 270 is located in the accommodation space 245 and is connected to the fifth movable seat 269 for driving the fifth movable seat 269 to move along the first additional direction D11 under the driving of the third driving device 225 of the second power assembly 14 so as to change the distance between the fourth roller 262 and the third roller 252. The third driving device may be configured to, for example, allow the fifth movable seat stay at any position within a continuous position range along the first additional direction D11. That is, the position of the fifth movable seat may be adjusted infinitely, so that the distance between the fourth roller 262 and the third roller 252 may be adjusted infinitely, and the guidewires or the catheters of different diameters may be better adapted.

The position adjustment assembly 270 includes, for example, an adjusting gear 272 and an adjusting rack 271. The adjusting gear 272 is located in the accommodation space 245 and is disposed at the second conveying assembly base 241 for rotating relative to the second conveying assembly base 241 under the driving of the third driving device 225 of the second power assembly 14. A rotation axis of the adjusting gear 272 is perpendicular to the first additional direction D11, e.g., parallel to the axial direction DA. The adjusting rack 271 is engaged with the adjusting gear 272, and the adjusting rack extends along the first additional direction D11 and is connected to the fifth movable seat 269. When the adjusting gear 272 is rotated, the adjusting rack 271 is moved along the first additional direction D11, and thus the fifth movable seat 269 is driven to move along the first additional direction D11.

The adjusting rack 271 is, for example, connected to a hole wall of one of the guide rod through holes 267, so that the position adjustment assembly 270 is located on one side of the fifth movable seat 269 and the fifth fixed seat 251, and does not interfere with the fifth movable seat 269 and the fifth fixed seat 251.

As shown in FIG. 29, the third driving device 225 includes a third driving component 226, a third transmission device (not shown) and a third driving shaft 227. The third driving component 226 is, for example, configured as a motor, e.g., a third servomotor. The third transmission device may be configured as a gear assembly, which is connected between an output shaft of the third servomotor and the third driving shaft 227, so that the third driving shaft 227 may be driven to rotate by the third servomotor may. When the second conveying assembly 60 is mounted to the second power assembly 14, the third driving shaft 227 is coaxially connected with the third rotating shaft 273 of the adjusting gear 272, so that the adjusting gear 272 is synchronously rotatable with the third driving shaft 227. The third rotating shaft 273 and the third driving shaft 227 may form a snap-fit connection with each other by the spline at the end. The third rotating shaft 273 may be disposed in a shaft bore of the second conveying assembly base 241 via a bearing. The adjusting gear 272 may be sleeved on the periphery of the third rotating shaft 273, and they are connected by the key and the keyway, so that they are coaxial and rotated synchronously. The adjusting gear 272 may also be integrally formed with the third rotating shaft 273.

The output shaft of the servomotor may rotate continuously and infinitely so that the position of the adjusting rack 271 may be infinitely adjusted to achieve a stepless adjustment of the distance between the fourth roller 262 and the third roller 252. Although the servomotor has been generally digitally controlled, the rotation angle of the servomotor has a high control accuracy (for example, relative to the stepper motor), so that the displacement of the rack 271 may be controlled at the micron level, and may be regarded as a stepless adjustment relative to the diameter of the guidewire or the catheter at the millimeter level in the present application.

The position adjustment assembly 270 is composed of rigid elements that, and may be located in a stable position under the action of the third driving device 225 so that a stable distance is maintained between the fourth roller 262 and the third roller 252, thus indicating that a sufficient clamping force may be provided by the third driving device 225 to clamp the guidewire and/or the catheter more firmly. The second conveying assembly 60 has more stable operating performance compared to the solution of using spring to pull the roller closer in the prior art. As shown in FIG. 30, the box cover 242 is provided with a second through hole 243 through which the third driving shaft 227 passes.

Thus, the first rotating shaft 253 is a first component of the third roller assembly 250 for connecting the fourth driving device 221, and the third rotating shaft 273 is a third component of the position adjustment assembly 270 for connecting the third driving device 225. Preferably, the first component and the third component are connected to the second power assembly 14 on the same side of the second conveying assembly 60. Preferably, the channel 231 (i.e., the gap between the third roller 252 and the fourth roller 262) and the ends of the first component (253) and the third component (273) connected to the second power assembly 14 are respectively located on different sides of the second conveying assembly 60, e.g., on opposite sides of the second conveying assembly 60 (the channel 231 is located on a side of the second conveying assembly 60 facing away from the second power assembly 14, and the ends of the first and third components connected to the second power assembly 14 are located on a side of the second conveying assembly 60 facing the second power assembly 14), thereby facilitating the installation of the guidewire 12 and/or the catheter 11. Preferably, the first rotating shaft 253 and the third rotating shaft 273 are arranged in parallel so that both of them may be connected to their respective driving devices at the same time, which is convenient for users to operate.

The second conveying assembly 60 may further include a biasing member 238 for applying a force to the fifth movable seat 269 to move away from the third roller 252 (i.e., the fifth fixed seat 251) along the first additional direction D11. The biasing member 238 may be disposed between the fifth movable seat 269 and the fifth fixed seat 251. In the illustrated embodiment, the biasing member 238 is configured as an elastic member, such as a spring. For example, the fifth movable seat 269 is provided with a mounting groove 268 for placing a spring 238, and the mounting groove 268 extends along the first additional direction D11. The fifth fixed seat 251 is provided with an acting portion 258, and the spring 238 abuts against or is connected to the acting portion 258 so as to be clamped by the fifth movable seat 269 and the fifth fixed seat 251.

The biasing member 238 assists the position adjustment assembly 270 to hold the fifth movable seat 269 in a stable position relative to the fifth fixed seat 251. The biasing member 238 also makes it difficult for the fourth roller 262 to contact the third roller 252. For example, it is possible for the biasing member 238 to make the channel 231 have a minimum width, that is, there may always be a gap between the third roller 252 and the fourth roller 262 to avoid direct contact between the third roller 252 and the fourth roller 262. This may protect the third roller 252 and the fourth roller 262, as well as the guidewire 12 and/or the catheter 11 in the channel 231. The biasing member 238 is used to increase the width of the channel 231, which facilitates the installation of the guidewire 12 and/or the catheter 11. For example, when the apparatus is not powered on, the fifth movable seat 269 may be pushed away from the fifth fixed seat 251 by the biasing member 238 to open the channel 231 and facilitate the installation of the guidewire 12 and/or the catheter 11.

In the embodiments not shown in the present application, the biasing member 238 includes a first magnet and a second magnet. The first magnet is disposed on a side of the fifth fixed seat 251 facing the fifth movable seat 269, and the second magnet is disposed on a side of the fifth movable seat 269 facing the fifth fixed seat 251. The first magnet and the second magnet are magnetically repulsive.

As described previously, the third roller 252 is configured as a driving roller. Preferably, the fourth roller 262 is also configured as a driving roller so that the second conveying assembly 60 may have sufficient conveying force. Preferably, the fourth roller 262 is driven by the third roller 252 to rotate about its own rotation axis rather than being driven by the second power assembly 14, which can simplify the transmission connection between the second conveying assembly 60 and the second power assembly 14.

In order to drive the fourth roller 262 to rotate, the second conveying assembly 60 further includes a roller transmission assembly 280. A first end of the roller transmission assembly 280 is connected to the third roller 252, and a second end thereof is connected to the fourth roller 262, so that the third roller 252 drives the fourth roller 262 to rotate in the opposite direction at the same rotational speed. In other words, the driving force provided by the third roller 252 is transferred to the fourth roller by the roller transmission assembly 280.

In a case where all the third rollers 252 are driving rollers, the first end of the roller transmission assembly 280 may be connected to any one of the third rollers 252. The fourth roller assembly 360 may include a plurality of fourth rollers 262, and the fourth roller assembly 360 is configured such that one of the fourth rollers 262 is used for rotating about its own axis under the driving of the third roller 252, and each of the other fourth rollers 262 is rotated about its own axis under the driving of another fourth roller 262. Thus, the roller transmission assembly 280 only needs to be connected with one fourth roller 262, and it is possible to simplify the connection between the roller transmission assembly 280 and the fourth roller assembly 360. At the same time, the first end of the roller transmission assembly 280 is also connected to only one third roller 252, which simplifies the connection between the roller transmission assembly 280 and the third roller assembly 250.

For example, as shown in FIG. 31, similar to the third roller assembly 250, the fourth roller assembly 360 may further include a plurality of second rotating shafts 263 and at least one second transmission belt 264. The second rotating shaft 263 is correspondingly arranged with the fourth roller 262 and is coaxially connected with the corresponding fourth roller 262. That is, each fourth roller 262 has its own second rotating shaft 263, and the two share a second axis P2. The fourth roller 262 may, for example, be sleeved on the periphery of the second rotating shaft 263, and they are connected by the key and the keyway so that they are synchronously rotatable. The fourth roller 262 may also be integrally formed with the second rotating shaft 263. The second rotating shaft 263 may be connected in a shaft hole of the fifth movable seat 269 via a bearing, so that the fourth roller 262 is connected to the fifth movable seat 269 through the second rotating shaft 263 and then connected to the second conveying assembly base 241 through the fifth movable seat 269. Wherein a part of the second rotating shafts 263 are used for rotating about its own axis P2 under the driving of the third roller 252, each second transmission belt 264 is connected to two second rotating shafts 263, and the other second rotating shafts 263 are driven to rotate by the second transmission belt 264. For example, a clamping ring is disposed around the periphery of the second rotating shaft 263, and the second transmission belt 264 is tensioned by the clamping rings of two second rotating shafts 263, so that the two second rotating shafts 263 are connected through the second transmission belt 264 to rotate synchronously. Thus, a part of the fourth rollers 262 are rotated under the driving of the third roller 252 through the roller transmission assembly 280, and the remaining fourth rollers 262 are each driven to rotate by another fourth roller 262 through the second transmission belt 264.

In the illustrated embodiment, one second rotating shaft 263 is used for rotating about its own axis P2 under the driving of the third roller 252, and the other second rotating shafts 263 are each driven to rotate through the second transmission belt 264. The number of the second transmission belts 264 is one less than the number of the fourth rollers 262, that is, one less than the number of the second rotating shafts 263. Thus, one fourth roller 262 is rotated under the driving of the third roller 252 through the roller transmission assembly 280, and the remaining fourth rollers 262 are each driven to rotate by another fourth roller 262 through the second transmission belt 264.

In the illustrated embodiment, the second conveying assembly 60 includes two fourth rollers 262 (i.e., the second conveying assembly 60 includes two second rotating shafts 263) and one second transmission belt 264. The two second rotating shafts 263 are connected by the second transmission belt 264. The two fourth rollers 262 are the first fourth roller 262A and the second fourth roller 262B, respectively. The second end of the roller transmission assembly 280 is connected to the second rotating shaft 263 of the second fourth roller 262B. Since the rotating shaft of the first third roller 252A is connected to the driving shaft 224, the roller transmission assembly 280 is more conveniently connected to the rotating shaft of the second third roller 252B. Further, the roller transmission assembly 280 is connected to the rotating shaft of the second fourth roller 262B in the vicinity. In other words, the Pth one of the third rollers 252 is used for rotating about its own axis P1 under the driving of the fourth driving device 221, the first end of the roller transmission assembly 280 is connected to the Qth one of the third rollers 252, the second end of the roller transmission assembly 280 is connected to the Qth one of the fourth rollers 262 (the Qth one of the third rollers 252 and the Qth one of the fourth roller 262 are a pair), and P is not equal to Q.

As shown in FIG. 30 to FIG. 34, the roller transmission assembly 280 may include N transmission gears 281 that are engaged sequentially. Wherein the first one of the transmission gears 281 is disposed at the first end of the roller transmission assembly 280 and is rotated synchronously with the third roller 252 (such as the second third roller 252B); the last one of the transmission gears 281 is disposed at the second end of the roller transmission assembly 280 and is rotated synchronously with the fourth roller 262 (such as the second third roller 252B). Since it is necessary for the second conveying assembly 60 to rotate the fourth roller 262 in the opposite direction to the third roller 252, the number N of the transmission gears 281 is an even number.

For example, similar to the adjusting gear 272, each transmission gear 281 has its own rotating shaft. A rotating shaft of the first one of the transmission gears 281 is coaxially connected to the first rotating shaft 253 of the third roller 252, and a rotating shaft of the last one of the transmission gears 281 is coaxially connected to the second rotating shaft 263 of the fourth roller 262, so that the power is transferred form the third roller 252 to the fourth roller 262 via a gear set.

In the case where the third roller 252 and the fourth roller 262 are configured as the same roller, the roller transmission assembly 280 has a transmission ratio 1:1. For example, all transmission gears 281 are configured as the same gear.

As described previously, the fifth movable seat 269 is movable along the first additional direction D11 relative to the fifth fixed seat 251. In order to allow the roller transmission assembly 280 to always be well connected to the fourth roller 262 and the third roller 252, the roller transmission assembly 280 is configured such that the second end of the roller transmission assembly 280 is movable relative to the first end of the roller transmission assembly 280 along the first additional direction D11, so as to adapt to the movement of the fifth movable seat 269 along the first additional direction D11. Specifically, the roller transmission assembly 280 is configured such that the last one of the transmission gears 281 is movable relative to the first one of the transmission gears 281 along the first additional direction D11. Since the relative position between the last one of the transmission gears 281 and the first one of the transmission gears 281 is not fixed, it cannot be guaranteed that the two are well connected at all times. Thus, the last one of the transmission gears 281 and the first one of the transmission gears 281 need to be connected through other transmission gears 281, and thus the number N of the transmission gears 281 is at least 4.

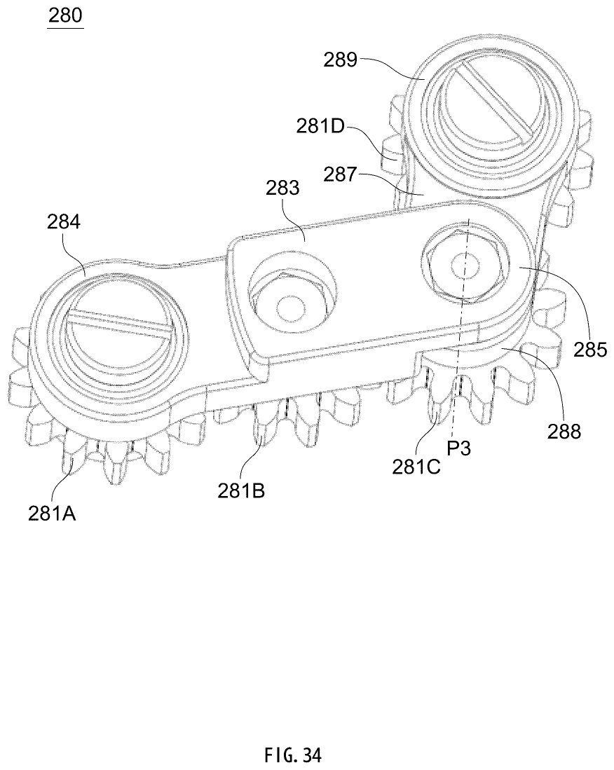

In order to achieve the extension and retraction of the roller transmission assembly 280 along the first additional direction D11, as shown in FIG. 34, the roller transmission assembly 280 also includes a first connecting arm 283 and a second connecting arm 287. The first connecting arm 283 includes a first end 284 of the first connecting arm and a second end 285 of the first connecting arm arranged in the opposite direction. The first end 284 of first connecting arm is the first end of the roller transmission assembly 280. The first one of the transmission gears 281 is disposed at the first end 284 of the first connecting arm. The second connecting arm 287 includes a first end 288 of the second connecting arm and a second end 289 of the second connecting arm arranged in the opposite direction. The second end 289 of the second connecting arm is the second end of the roller transmission assembly 280. The last one of the transmission gears 281 is disposed at the second end 289 of the second connecting arm. The N transmission gears 281 are disposed at the first connecting arm 283 and the second connecting arm 287. Wherein the first end 288 of the second connecting arm is rotatably connected to the second end 285 of the first connecting arm about a third axis P3, and the third axis P3 is parallel to the axis P1 of the third roller 252 (also parallel to the axis P2 of the fourth roller 262).

The first connecting arm 283 and the second connecting arm 287 are rotatably connected so that the roller transmission assembly 280 may be bent, thereby changing the distance between the first end and the second end of the roller transmission assembly 280.

The rotating shaft of each transmission gear 281 is mounted into a shaft hole of the first connecting arm 283 or the second connecting arm 287, for example via a bearing, so that the transmission gear 281 is rotatable relative to the respective connecting arms.

In order to ensure that N transmission gears 281 are always well connected, it is necessary to ensure that the distance between the axes of two adjacent transmission gears 281 is constant during the bending of the roller transmission assembly 280. Therefore, the roller transmission assembly 280 is configured such that the third axis P3 coincides with an axis of one transmission gear 281 other than the first one of the transmission gears 281 and the last one of the transmission gears 281. In other words, one transmission gear 281 other than the first one of the transmission gears 281 and the last one of the transmission gears 281 is simultaneously positioned at the second end 285 of the first connecting arm and the first end 288 of the second connecting arm. The second end 285 of the first connecting arm and the first end 288 of the second connecting arm may be rotatably connected through the rotating shaft of the one transmission gear 281, so that the second end 285 of the first connecting arm and the first end 288 of the second connecting arm are rotatable relative to each other about the axis of the one transmission gear 281.

In the illustrated embodiment, the roller transmission assembly 280 includes four transmission gears 281, which are the first transmission gear 281A, the second transmission gear 281B, the third transmission gear 281C and the fourth transmission gear 281D, respectively. The fourth transmission gear 281D is also the last one of the transmission gears. The four transmission gears 281 are engaged sequentially. Wherein the first transmission gear 281A and the second transmission gear 281B are disposed at the first connecting arm 283, the third transmission gear 281C is simultaneously positioned at the second end 285 of the first connecting arm and the first end 288 of the second connecting arm, and the second end 285 of the first connecting arm and the first end 288 of the second connecting arm are rotatably connected through the rotating shaft of the third transmission gear 281C. Thus, the first transmission gear 281A, the second transmission gear 281B and the third transmission gear 281C are all disposed at the first connecting arm 283, and the relative positions of the three transmission gears 281 are fixed. The third transmission gear 281C and the fourth transmission gear 281D are both disposed at the second connecting arm 287, and the relative positions of the two transmission gears 281 are fixed. In this way, the distance between the two adjacent transmission gears 281 is always constant when the first connecting arm 283 and the second connecting arm 287 are rotated relatively, and all the transmission gears 281 may be well engaged all the time.

Of course, the second end 285 of the first connecting arm and the first end 288 of the second connecting arm may also be rotatably connected by the rotating shaft of the second transmission gear 281B.

In the illustrated embodiment, the first additional direction D11 is an up-down direction so that the third roller 252 and the fourth roller 262 are placed horizontally. When the guidewire and/or the catheter is installed, the roller located below may act as a support for the guidewire and/or the catheter, facilitating the placement of the guidewire and/or the catheter in the channel 231. The rollers are horizontally placed so that the second power assembly 14 (or the driving device) may be disposed on the side of the second conveying assembly 60 (also the rollers). Compared to the solution in the prior art where the roller is placed vertically and the driving device is located below the roller, the second conveying assembly 60 is more convenient to be close to the surgical patient. Thus, it is possible to reduce the lengths of the guidewire and the catheter. In particular, when the second conveying assembly 60 is mounted with the large sheath 16, the second conveying assembly 60, for example, is used for artificial heart valve implantation surgery. Reducing the lengths of the guidewire and the catheter to facilitate the surgery operation is of great significance for ensuring the quality of the surgery.

In the illustrated embodiment, the third roller 252 is located above the fourth roller 262 so that it may be designed such that the second conveying assembly 60 is connected only with the lower half of the second power assembly 14 and not extend to the upper half of the second power assembly 14. In this way, most of the structure of the second power assembly 14 is located above the second conveying assembly 60, and it is possible to dispose a detachable cover of the housing of the second power assembly 14 above the second conveying assembly 60 and facilitate the overhaul of the second power assembly 14. Furthermore, the second power assembly 14 is only connected to the upper half of the second conveying assembly 60 and does not extend to the lower half of the second conveying assembly 60, which facilitates the second conveying assembly 60 to be closer to the surgical bed. At the same time, when the apparatus is not powered on, the fourth roller 262 may, to a certain extent, rely on its own gravity to move the fifth movable seat 262 downward, assisting the biasing member 238 to open the channel 231.

Of course, the fourth roller 262 may also be located above the third roller 252. In this way, when the guidewire or the catheter is installed, the guidewire or the catheter is supported by the third roller 252, and the third roller 252 does not move, which facilitates the guidewire and the catheter to stay stably in the channel 231.

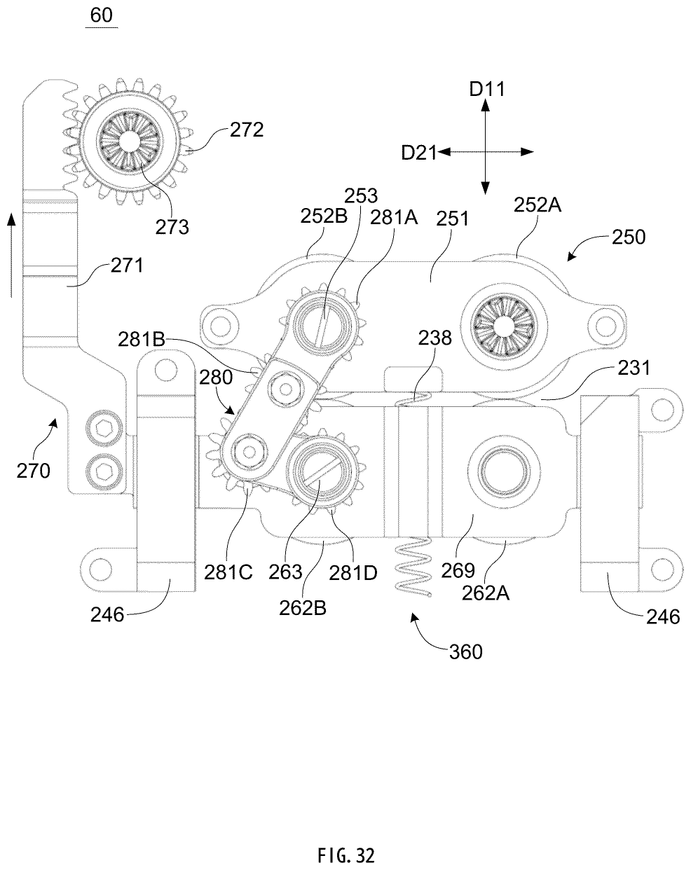

In actual use, the second power assembly 14 is first mounted to the second supporting frame 19 by the user, the second conveying assembly 60 is then mounted to the second power assembly 14, the catheter 11 and/or the guidewire 12 are then installed to the second conveying assembly 60, and then the apparatus is started. The catheter 11 and/or the guidewire 12 are first clamped by the second conveying assembly 60. Specifically, the third driving device 225 further includes a third servomotor driver for controlling the operation of the third servomotor. The third servomotor driver is configured to drive the third servomotor to rotate under the control of the host computer (control module) and monitor the operating current of the third servomotor. After the third servomotor is rotated, the fifth movable seat 269 is moved towards the fifth fixed seat 251, and the channel 231 is gradually narrowed (as shown in FIG. 32). When the channel 231 is subjected to the resistance from the catheter 11 and/or guidewire 12, it indicates that the catheter 11 and/or guidewire 12 have been clamped, and the operating current of the third servomotor has increased significantly at this time. The medical system 300 is configured to stop the operation of the third servomotor and complete the clamping step when the operating current of the third servomotor reaches a preset threshold.

The preset threshold of the operating current of the third servomotor may be obtained from a large number of tests by clamping various types of guidewires and/or catheters. Also, different preset thresholds may be set depending on the diameter and/or material of various guidewire (or catheter). For example, for a catheter of the same caliber, when the material of the catheter is soft, a clamping force output by the third servomotor is relatively small (the preset threshold is relatively low) to avoid the catheter being over-clamped and deformed; when the material of the catheter is hard, the clamping force output by the third servomotor is relatively large (the preset threshold is relatively high) to avoid insufficient clamping force.

After the guidewire 12 and/or the catheter 11 are clamped, the convey operation may be performed. The slave-side execution apparatus 200 is configured to cause the fourth driving device 221 to operate after the third servomotor is stopped from operating. Similarly, for example, the fourth driving device 221 further includes a fourth servomotor driver for controlling the operation of the fourth servomotor. After the third servomotor is stopped from operating, the host computer controls the fourth servomotor driver to rotate the fourth servomotor to allow the fourth driving device 221 to operate and the fourth driving shaft 224 to rotate.

During the normal convey of the guidewire 12 and/or the catheter 11, the guidewire 12 and/or the catheter 11 are interacted with the rollers through static friction, and the cumulative rotation distance of the outer surface of the roller shall be equivalent to the conveyed length of the guidewire 12 and/or the catheter 11. The apparatus may be provided with a sensor to monitor the conveyed length of the guidewire 12 and/or the catheter 11, or to estimate the conveyed length of the guidewire 12 and/or the catheter 11 by imaging. When the rotation distance of the outer surface of the roller corresponding to the cumulative rotation angle of the fourth servomotor does not match the conveyed length of the guidewire 12 and/or the catheter 11, it indicates that the guidewire 12 and/or the catheter 11 are not clamped. At this time, the rotation angle or output power of the third servomotor may be increased to clamp the guidewire 12 and/or the catheter 11.

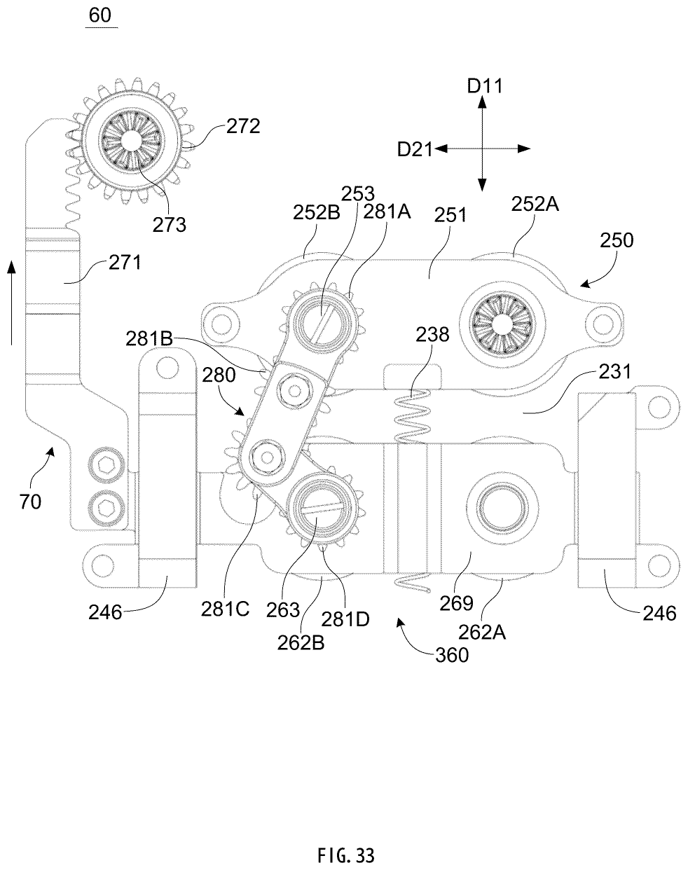

The third driving device 225 is used for controlling the distance between the fifth movable seat 269 and the fifth fixed seat 251. It may be understood that when the guidewire 12 and/or the catheter 11 need to be detached, the third driving shaft 227 is rotated to move the fifth movable seat 269 away from the fifth fixed seat 251 (as shown in FIG. 33). When the fifth movable seat 269 is moved away from the fifth fixed seat 251, the spring 238 is elongated. When the length of the spring 238 exceeds the free length, the spring 238 applies a force to the fifth movable seat 269 to bring it close to the fifth fixed seat 251. At this time, the resistance of the fifth movable seat 269 to move away from the fifth fixed seat 251 increases, causing the operating current of the third servomotor to increase. Therefore, in the reverse rotation, it is also possible to determine whether the channel 231 has been opened to a suitable degree based on the operating current of the third servomotor, and for example, the third servomotor may be stopped from operating when the operating current of the third servomotor reaches a reverse preset threshold. Of course, the channel 231 may also be fully opened by the biasing member 238 when the apparatus is powered off.

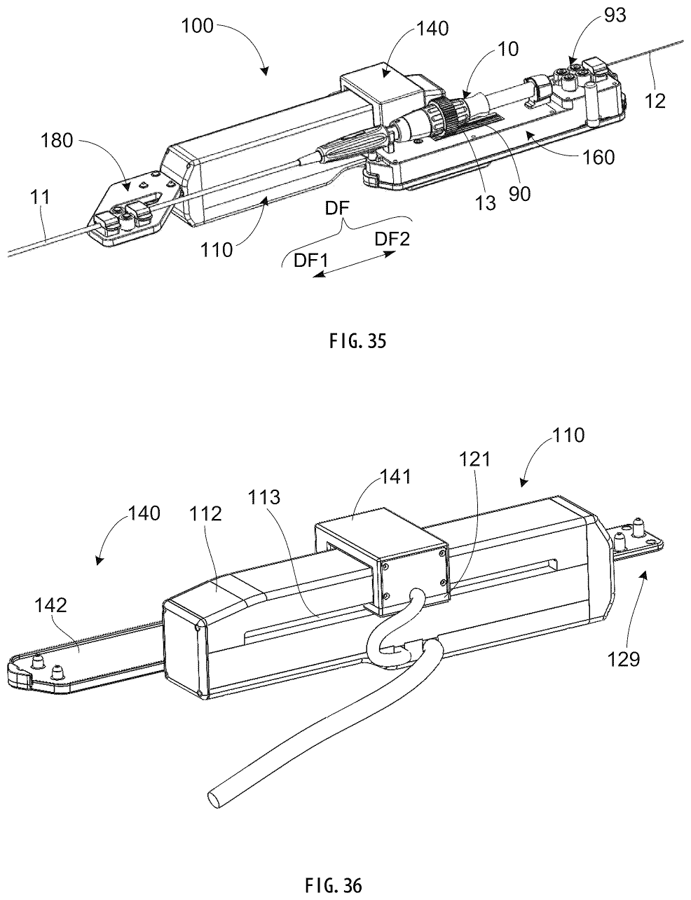

As shown in FIG. 35, in a second embodiment of the present application, the configuration of the slave-side execution apparatus 100 differs from that of the slave-side execution apparatus 200. The following highlights only the differences between the two embodiments.

The slave-side execution apparatus 100 includes a main body power device 110, a movable power device 140, and a first carrying device 160.

The main body power device 110 has a main body driving device and a movable assembly. The movable assembly is configured to be movable in the first conveying direction DF1 or the second conveying direction DF2 under the action of the main body driving device. The first conveying direction DF1 is the advancing direction of the guidewire 12 or the catheter 11, and the second conveying direction DF2 is the retracting direction of the guidewire 12 or the catheter 11. In other words, the movable assembly is drivingly connected to the main body driving device to be movable forward and backward along the conveying direction DF under the action of the main body driving device 124.

The movable power device 140 is connected to the movable assembly and is configured to be movable with the movable assembly along the first conveying direction DF1 or the second conveying direction DF2. Moreover, the movable power device 140 has a first driving device. The first carrying device 160 is disposed on the movable power device 140 and is used to carry the medical apparatus 10. Thus, the first carrying device 160 is capable of carrying the medical apparatus 10 to move with the movable power device 140 along the first conveying direction DF1 or the second conveying direction DF2 to perform the advancing action or the retracting action of the catheter 11.

The first carrying device 160 is provided with a first manipulation device 93. Different form the first embodiment, the first manipulation device 93 is integrated with the first carrying device 160. Therefore, in the second embodiment, the first manipulation device 93 may be regarded as a part of the first carrying device 160. That is, the first carrying device 160 has the first manipulation device 93. The first manipulation device 93 is in transmission connection with the first driving device so that the advancing action or retracting action of the guidewire 12 may be manipulated by the first manipulation device 93 under the action of the first driving device.

The first carrying device 160 further includes a second manipulation device 90 for acting on the medical function mechanism 13 of the medical apparatus 10. The movable power device 140 further includes a second driving device for driving the second manipulation device 90 to operate.

As shown in FIG. 36 to FIG. 38, the main body power device 110 further includes a machine frame housing 112. The housing 112 has a long hole 113 extending along the first conveying direction DF1 or the second conveying direction DF2. The protrusion 121 of the movable assembly extends from the long hole 113. The movable power device 140 further includes a connecting frame 141 and a base 142. The connecting frame 141 may be substantially configured in a U shape to be sleeved on the upper side of the housing 112. Wherein one end of the connecting frame 141 is connected to the base 142, and the other end is connected to the protrusion 121 of the movable assembly, so that the connecting frame 141 is movable across the housing 112.

The first carrying device 160 is detachably connected to the base 142. In the second embodiment, the base 142 is used to extend substantially along a horizontal plane, and the first carrying device 160 is mounted to the upper side of the base 142, which may also be understood that the first carrying device 160 is used horizontally.

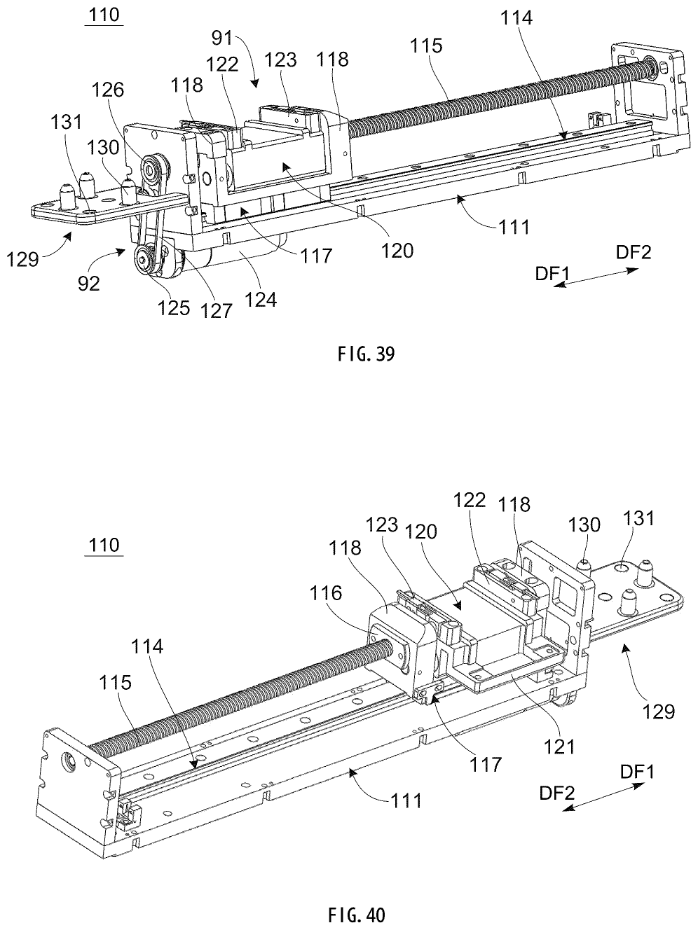

As shown in FIGS. 39 and 40, similar to the first embodiment, the main body power device 110 further includes a machine frame 111, a first guide rail 114, a lead screw pair (the lead screw pair includes a lead screw 115 and a lead screw nut 116) and a main body transmission assembly 92. The housing 112 covers the machine frame 111. The main body driving device 124, the movable assembly 91, the first guide rail 114, the lead screw pair and the main body transmission assembly 92 are all disposed on the machine frame 111.

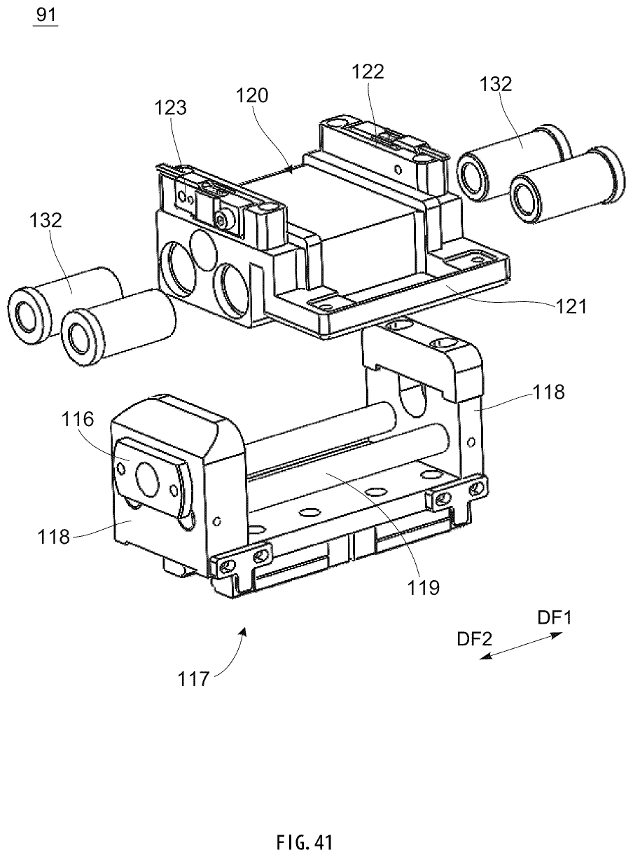

The movable frame 117 has two end walls 118 and a second guide rail 119 disposed between the two end walls 118. The movable seat 120 is preferably disposed on the second guide rail 119 via linear bearing(s) 132. The movable seat 120 has the protrusion 121. In the second embodiment, the configuration of the movable assembly 91 is different from that of the movable assembly 291. As shown in FIG. 41, a first force sensing member 122 and a second force sensing member 123 are respectively disposed at both ends of the movable seat 120. The first force sensing member 122 and the second force sensing member 123 are respectively disposed facing the sides of the two end walls 118 of the movable frame 11 and respectively abut against the corresponding end wall 118. Wherein the first force sensing member 122 is closer to the end (front end) of the main body power device 110 along the first conveying direction DF1 than the second force sensing member 123. Therefore, when the advancing action of the catheter 11 is performed, the first force sensing member 122 may sense the resistance experienced by the movable seat 120. Wherein the first force sensing member 122 and the second force sensing member 123 are preferably pressure sensors or weighing sensors.

As shown in FIGS. 42 and 43, similar to the first embodiment, the movable power device 140 further includes a first power shaft 145, a first transmission assembly 94, a second power shaft 146, and a second transmission assembly 95.

The first driving device 143 and the second driving device 144 are disposed on the base 142. Preferably, the first driving device 143 and the second driving device 144 are at least partially located in the connecting frame 141, and the driving ends of the two may extend to the bottom of the base 142.

The first power shaft 145 and the second power shaft 146 are arranged to penetrate through the base 142. The top end of the first power shaft 145 is in transmission connection with the first manipulation device 93, and the bottom end is connected to the first transmission assembly 94. Moreover, the first transmission assembly is also connected to the driving end of the first driving device 143 so as to transfer power from the first driving device 143 to the first power shaft 145 and then to the first manipulation device 93. The top end of the second power shaft 146 is in transmission connection with to the second manipulation device 90, and the bottom end is connected to the second transmission assembly 95. Moreover, the second transmission assembly 95 is also connected to the driving end of the second driving device 144 so as to transfer power from the second driving device 144 to the second power shaft 146 and then to the second manipulation device 90.

Wherein, the first transmission assembly 94 includes the first driving roller 147, the first driven roller 148 and the first synchronous belt 149. The second transmission assembly 95 includes the second driving roller 150, the second driven roller 151 and the second synchronous belt 152. The first transmission assembly 94 and the second transmission assembly 95 are configured in the same manner as in the first embodiment, which will not be described in detail herein.

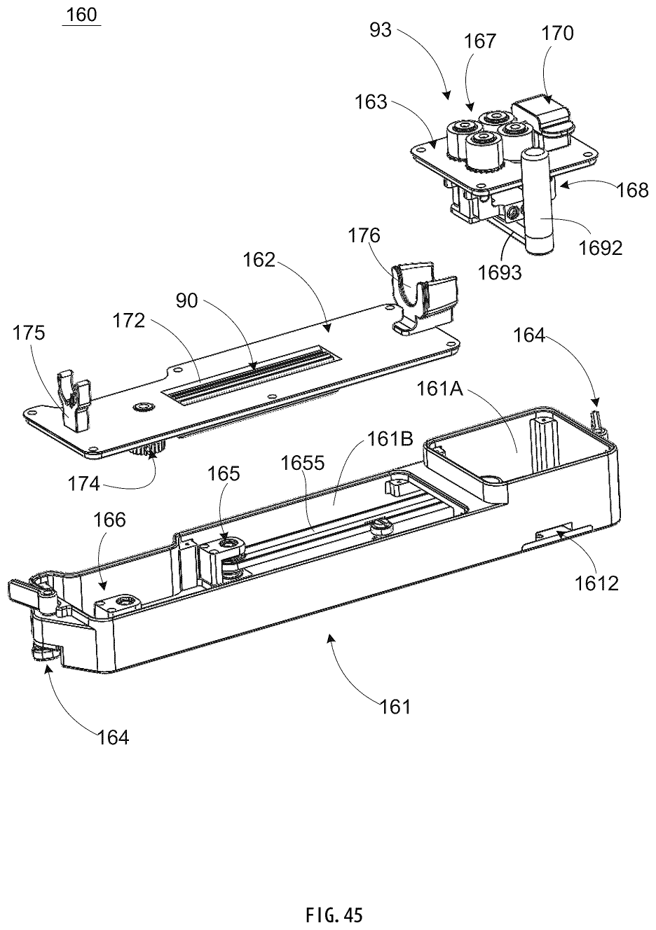

The structure of the first carrying device 160 will be described below in conjunction with FIGS. 44 to 47.

The first carrying device 160 includes a carrying base 161, a guidewire manipulation seat 163 and a functional manipulation seat 162. Wherein the carrying base 161 is used to be mounted to the base 142 of the movable power device 140. The carrying base 161 is substantially configured in the shape of a box, including a first part 161A of the carrying base for mounting the first manipulation device 93 and a second part 161B of the carrying base for mounting the second manipulation device 90. The first part 161A of the carrying base and the second part 161B of the carrying base are both box-shaped. The guidewire manipulation seat 163 is disposed on the carrying base 161. The guidewire manipulation seat 163 is substantially plate-shaped for covering the first part 161A of the carrying base, and the first manipulation device 93 is disposed on the guidewire manipulation seat 163. The functional manipulation seat 162 is disposed on the carrying base 161. The functional manipulation seat 162 is substantially plate-shaped for covering the second part 161B of the carrying base, and the second manipulation device 90 is disposed on the functional manipulation seat 162.

In the second embodiment, the structure of the carrying base 161 is different from that of the carrying base 261, and the first manipulation device 93 and the medical apparatus 10 are in an exposed state during use.

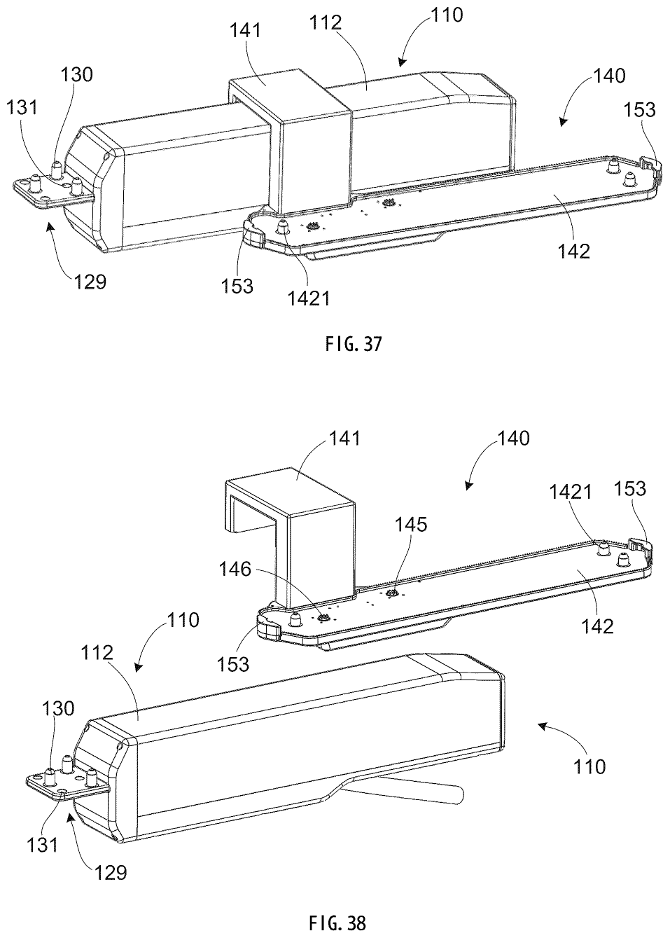

Similar to the first embodiment, the snapping assembly 164 disposed at the end of the carrying base 161 is connectable to the buckle seat 153 (as shown in FIG. 37) disposed at the end of the base 142 of the movable power device 140, so that the carrying base 161 is detachably connected to the base 142.

In order to further facilitate the rapid mounting and positioning of the first carrying device 160, a connecting mating part 1611 is provided on the carrying base 161, and a connecting part 1421 is provided on the base 142 of the movable power device 140 (as shown in FIGS. 37 and 38), wherein the connecting part 1421 is capable of forming a mating connection with the connecting mating part 1611. Exemplarily, the connecting part 1421 may be a connecting column, and the connecting mating part 1611 may have a corresponding connecting hole. When the first carrying device 160 is mounted, the mounting may be quickly completed by simply positioning the connecting column and the connecting hole.

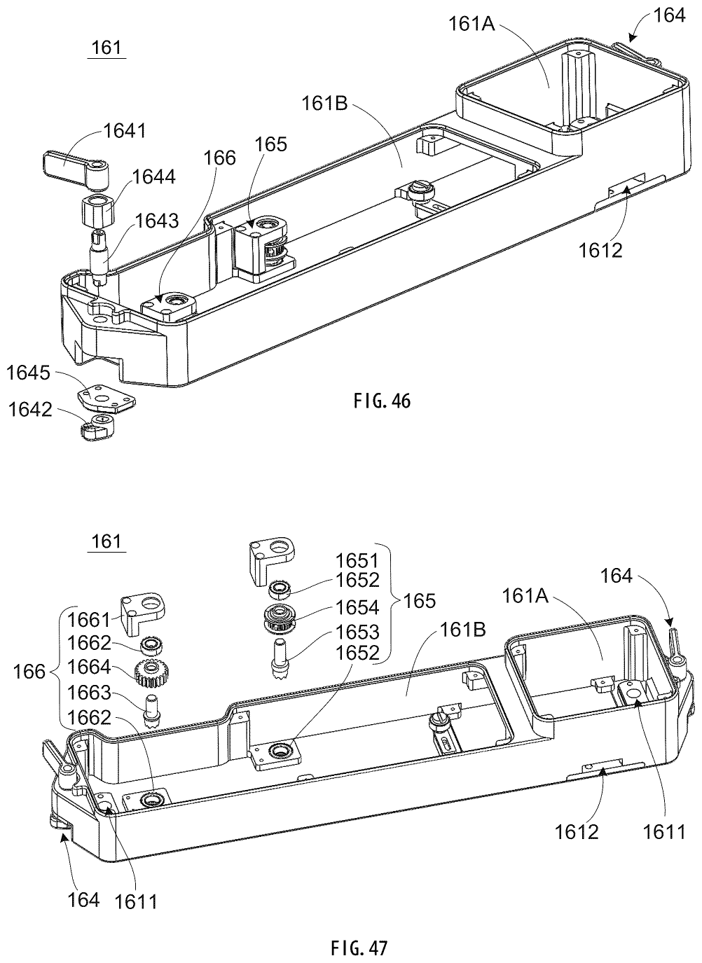

With reference to FIGS. 46 and 47, a first power transfer device 165 and a second power transfer device 166 are disposed in the carrying base 161. Wherein the first power transfer device 165 is located at a position corresponding to the position of the first power shaft 145 and is engaged with the first power shaft 145 so as to transfer the rotational motion of the first power shaft 145 to the first manipulation device 93. The second power transfer device 166 is located at a position corresponding to the position of the second power shaft 146 and is engaged with the second power shaft 146 so as to transfer the rotational motion of the second power shaft 146 to the second manipulation device 90.

Specifically, the first power transfer device 165 includes the first limiting frame 1651, the first bearing 1652, the first transmission shaft 1653, the transfer synchronous roller 1654, and a transfer synchronous belt 1655. A hole corresponding to the first power shaft 145 is provided at the bottom of the carrying base 161, the first limiting frame 1651 is provided on the carrying base 161 and corresponds to the hole, and two first bearings 1652 corresponding to each other are provided at the hole and on the first limiting frame 1651. The first transmission shaft 1653 is connected between the inner rings of the two first bearings 1652, and the bottom end of the first transmission shaft 1653 is engaged with the first power shaft 145, the upper part of the first transmission shaft 1653 is fixedly connected to the transfer synchronous roller 1654. The transfer synchronous belt 1655 is connected between the transfer synchronous roller 1654 and the first manipulation device, so that a synchronous belt transmission is formed between the first power transfer device 165 and the first manipulation device 93.

The second power transfer device 166 includes a second limiting frame 1661, a second bearing 1663, a second transmission shaft 1662, and a transfer gear 1664. A hole corresponding to the second power shaft 146 is also provided at the bottom of the carrying base 161. The second limiting frame 1661 is provided on the carrying base 161 and corresponds to the hole, and two second bearings 1663 corresponding to each other are provided at the hole and on the second limiting frame 1661. The second transmission shaft 1662 is connected between the inner rings of the two second bearings 1663, and the bottom end of the second transmission shaft 1662 is engaged with the second power shaft 146, the upper part of the second transmission shaft 1662 is fixedly connected to the transfer gear 1664. A gear engaging transmission is formed between the second power transfer device 166 and the second manipulation device 90.

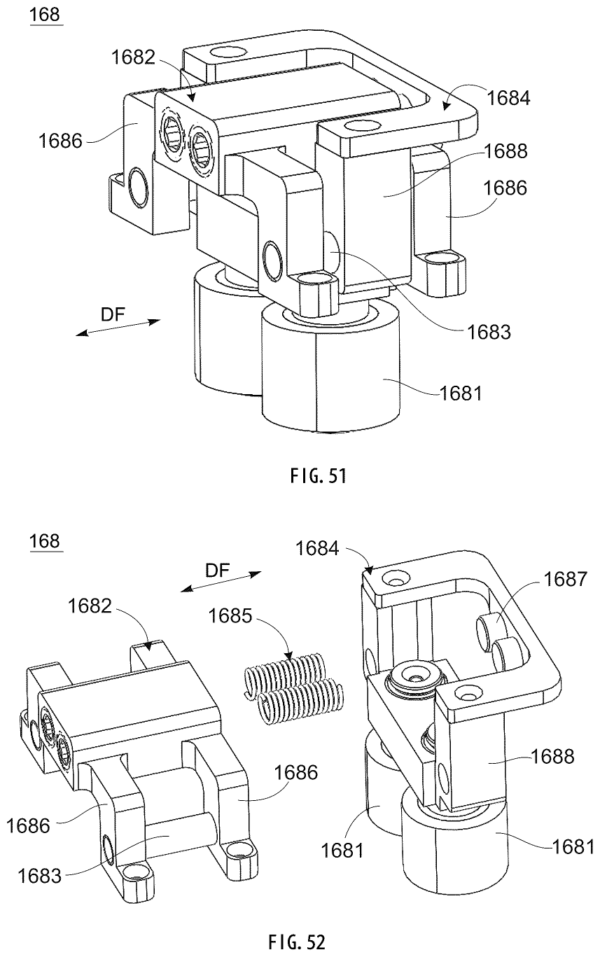

The specific structure of the first manipulation device 93 will be described below in conjunction with FIGS. 48 to 52.

The first manipulation device 93 includes a driving roller member 167 and a driven roller member 168. The driving roller member 167 and the driven roller member 168 are arranged opposite to each other. The driving roller member 167 includes a driving roller 1671 disposed on the upper side of the guidewire manipulation seat 163 and a driving gear 1672 disposed on the lower side of the guidewire manipulation seat 163. The driving roller 1671 and the driving gear 1672 are coaxially fixedly connected, and the driving gear 1672 is in transmission connection with the first power transfer device 165 so as to rotate about its own axis relative to the first carrying device 160 under the action of the first power transfer device 165.

Wherein, a transmission gear 1673 is also provided on the bottom side of the guidewire manipulation seat 163, and the driving gear 1672 corresponding to the driving roller 1671 is engaged with the transmission gear 1673. A transmission synchronous roller 1674 is coaxially provided with the transmission gear 1673. A synchronous belt transmission is formed between the transmission synchronous roller 1674 and the transfer synchronous roller 1654, thereby realizing a transmission connection between the driving gear 1672 and the first power transfer device 165. Preferably, the driving rollers 1671 are also set to two, and the two driving gears 1672 of the two driving rollers 1671 are both engaged with the transmission gear 1673.

The driven roller member 168 is disposed on the guidewire manipulation seat 163 and includes a driven roller 1681 located on the upper side of the guidewire manipulation seat 163. The driven roller 1681 is substantially parallel to the driving roller 1671. The driven roller 1681 is arranged opposite to the driving roller 1671 along the first direction D1, and the first direction D1 is perpendicular to a plane determined by the conveying direction DF and an axis of the driven roller 1681. The driven roller 1681 is configured to be movable in a direction close to the driving roller 1671 or away from the driving roller 1671. That is, the driven roller 1681 is movable relative to the driving roller 1671 along the first direction D1, so as to be able to clamp or release the guidewire 12 with the driving roller 1671. The number of the driven rollers 1681 corresponds to that of the driving rollers 1671. For example in a preferred embodiment, the driven rollers 1681 are also set to two.

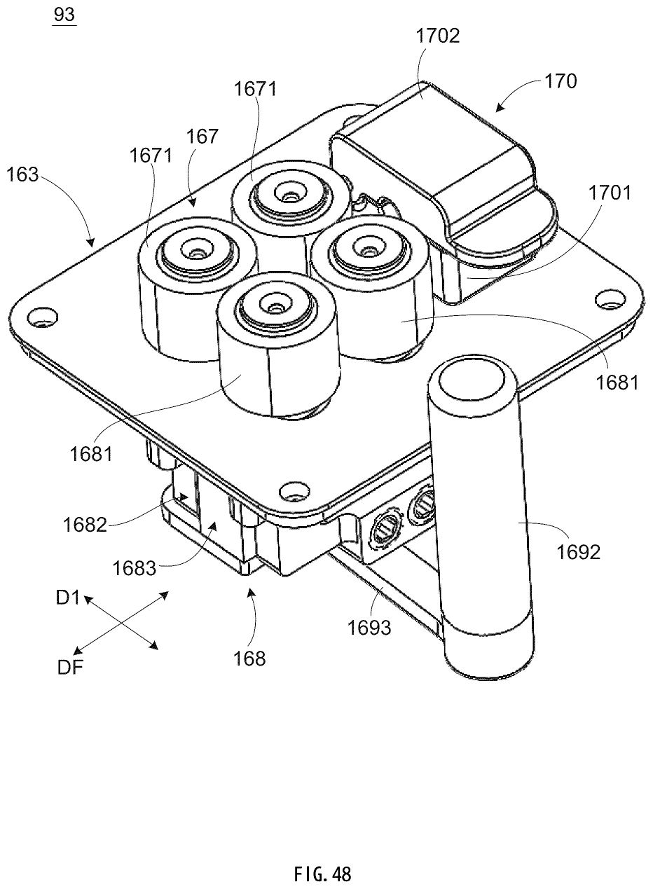

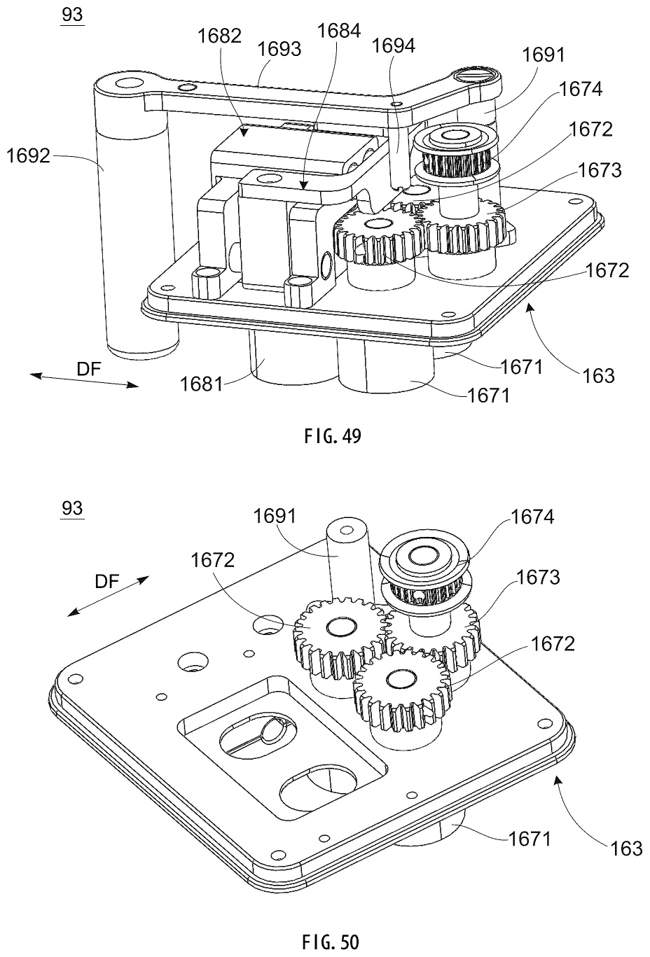

With reference to FIGS. 49 to 52, the driven roller member 168 further includes a fixed frame 1682, an extrusion frame 1684, and a first elastic member 1685.

Wherein, the fixed frame 1682 is connected to the guidewire manipulation seat 163 and has a pair of arm portions 1686 extending out, and the pair of arm portions 1686 are separated from each other. Preferably, a third guide rail 1683 is provided between the pair of the two arm portions 1686, and the third guide rail 1683 extends along the first direction D1. Further preferably, two pairs of arm portions 1686 extending in opposite directions are provided on both sides of the fixed frame 1682, and one third guide rail 1683 is provided between the two arm portions 1686 of each pair.

The extrusion frame 1684 has a sliding portion 1688 on which the driven roller 1681 is provided, and the sliding portion 1688 is disposed on the fixed frame 1682. Wherein the third guide rail 1683 is arranged to penetrate through the sliding portion 1688 so that the extrusion frame 1684 is movable along the third guide rail 1683. Wherein the sliding portion 1688 is located between the pair of arm portions 1686 of the fixed frame 1682. Preferably, the two sliding portions 1688 are respectively located between the two pairs of arm portions 1686. When the extrusion frame 1684 is mounted on the fixed frame 1682, the fixed frame 1682 is semi-enclosed by the extrusion frame 1684.

The first elastic member 1685 is connected between the fixed frame 1682 and the extrusion frame 1684, and extends along the first direction D1 to provide an elastic force to the extrusion frame 1684. The elastic force causes the extrusion frame 1684 to tend to move in the direction close to the driving roller 1671. That is, the first elastic member 1685 is used to provide a force that brings the extrusion frame 1684 close to the driving roller 1671 along the first direction D1. In the illustrated embodiment, the first elastic member 1685 is configured as a spring and may be disposed in a cavity within the fixed frame 1682. An extrusion portion 1687 is provided on the extrusion frame 1684 at a position corresponding to the cavity. When the extrusion frame 1684 is mounted to the fixed frame 1682, the extrusion portion 1687 may partially extend into the cavity, and the first elastic member 1685 abuts against both the extrusion portion 1687 and the fixed frame 1682 in the cavity.

With reference to FIGS. 48 and 49, a manipulation rod assembly 169 is also provided on the guidewire manipulation seat 163. The manipulation rod assembly 169 includes a pivoting portion 1691, a manipulation portion 1692, an extension portion 1693, and a pushing portion 1694.

Wherein, the pivoting portion 1691 is pivotally connected to the guidewire manipulation seat 163. That is, the pivoting portion 1691 is connected to the guidewire manipulation seat 163 and is rotatable about its own axis relative to the guidewire manipulation seat 163. An adjustment opening 1612 (see FIG. 44) is provided on the carrying base 161. The extension portion 1693 is located at the lower side of the guidewire manipulation seat 163, one end of which is connected to the pivoting portion 1691, and the other end passes through the adjustment opening 1621 and extends to the outside of the carrying base 161. In the illustrated embodiment, the extension portion 1693 may have a certain bending angle, for example, between 50° and 130°, or 90°, etc. The manipulation portion 1692 is used for being operated by a user, which is located outside the carrying base 161 and connected to the other end of the extension portion 1693. The extension portion 1692 is connected between the pivoting portion 1691 and the manipulation portion 1692 so that the manipulation portion 1692 is capable of rotating synchronously with the pivoting portion 1691 about an axis of the pivoting portion 1691.

The pushing portion 1694 is provided to the extension portion 1693 for contacting the extrusion frame 1684 to enable the pushing portion 1694 to push the extrusion frame 1684 away from the driving roller 1671 along the first direction D1 when the pivoting portion 1691 is rotated. For example, the pushing portion 1694 is located in the carrying base 161 and extends from the extension portion 1693 towards the guidewire manipulation seat 163. In the illustrated embodiment, the pushing portion 1694 is connected to the bend of the extension portion 1693. Along a moving direction of the extrusion frame 1684 under the action of the first elastic member 1685, the pushing portion 1694 is located at the downstream side relative to the extrusion frame 1684, so that when the pivoting portion 1691 is pivoted, the pushing portion 1694 is able to push the extrusion frame 1684 to cause the driven roller 1681 to move away from the driving roller 1671. Exemplarily, when the manipulation portion 1692 is manipulated by an operator, the extrusion frame 1684 may be pushed by the pushing portion 1694 to move in a direction close to the fixed frame 1682. This enables the driven roller 1681 to move away from the driving roller 1671, thereby releasing the clamping of the driven roller 1681 and the driving roller 1671 on the guidewire 12.

In a second embodiment, the upper side of the guidewire manipulation seat 163 is further provided with a guidewire supporting device 170, which is configured to allow the guidewire 12 to pass through to support the guidewire 12. As shown in FIG. 48, the guidewire supporting device 170 includes a guidewire supporting portion 1701 and a guidewire cover portion 1702. The guidewire supporting portion 1701 is connected to the guidewire manipulation seat 163. A semi-guidewire groove is provided on the guidewire supporting portion 1701. The guidewire cover portion 1702 may be connected to the guidewire supporting portion 1701 in an openable and closable manner, and a semi-guidewire groove is also provided on the guidewire cover portion 1702. When the guidewire cover portion 1702 covers the guidewire supporting portion 1701, the two semi-guidewire grooves form a complete guidewire groove to support the guidewire 12 and allow the guidewire 12 to pass through. Preferably, a snapping structure may be provided on the guidewire supporting portion 1701 and the guidewire cover portion 1702 to form a snap-fit connection when the guidewire cover portion 1702 covers the guidewire supporting portion 1701.

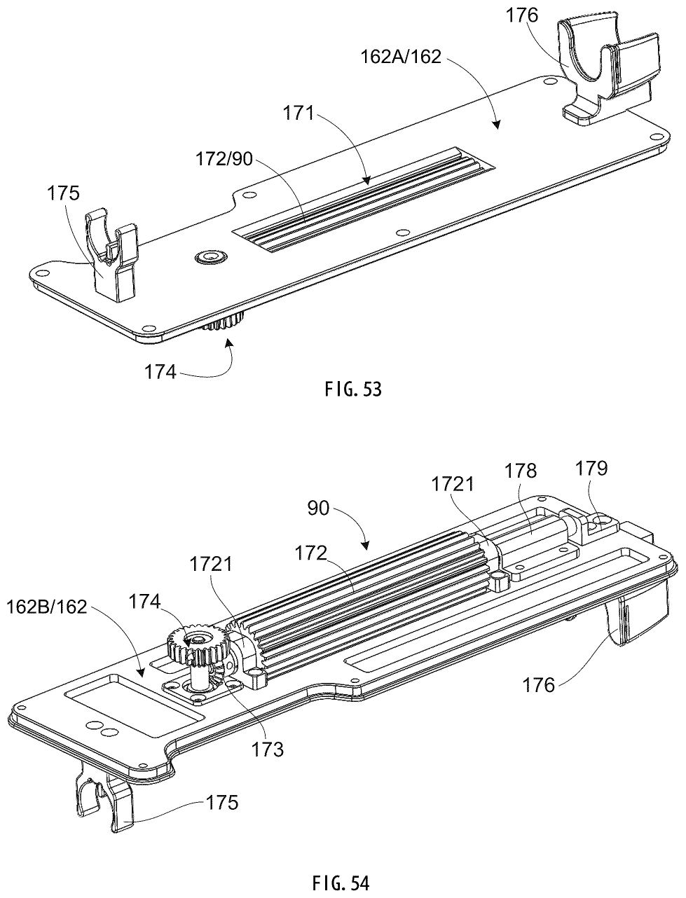

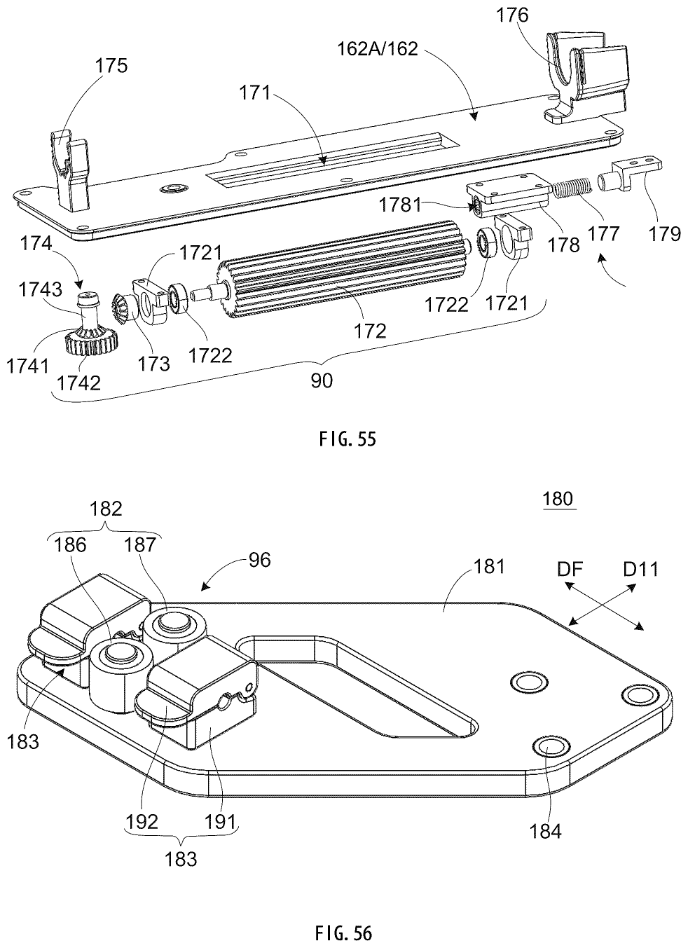

The specific structures of the functional manipulation seat 162 and the second manipulation device 90 will be introduced below in conjunction with FIGS. 53 to 55.

The first carrying device 160 has a supporting structure 97 capable of supporting the medical apparatus 10. Specifically, similar to the first embodiment, the supporting structure 97 includes the first bracket 175, the second bracket 176, the second elastic member 177, the connecting seat 178 and the bottom bracket 179, which will not be described in detail herein.

The second manipulation device 90 includes a functional gear 172, a bevel gear 173 and a bevel spur gear 174. Wherein the functional gear 172 is rotatably disposed on the functional manipulation seat 162 and is at least partially located on the upper side of the functional manipulation seat 162 (the first side 162A of the functional manipulation seat). Exemplarily, the functional gear 172 is disposed on the lower side of the functional manipulation seat 162 (the second side 162B of the functional manipulation seat). The functional manipulation seat 162 is provided with an operation opening 171 through which the functional gear 172 is partially exposed to the upper side of the functional manipulation seat 162. Thus, when the medical apparatus 10 is mounted to the first carrying device 160, the medical function mechanism 13 of the medical apparatus 10 is engageable with the functional gear 172.

In an optional embodiment, the bottom side of the functional manipulation seat 162 is provided with two gear seats 1721 at both ends of the operation opening 171. The two gear seats 1721 are respectively provided with one third bearing 1722, and the two ends of the rotating shaft of the functional gear 172 are fixedly connected to the inner rings of the two third bearings 1722, respectively. Moreover, one end of the rotating shaft of the functional gear 172 is also fixedly connected to the bevel gear 173 so that the functional gear 172 and the bevel gear 173 are coaxially rotatable. The bevel spur gear 174 is rotatably connected to the functional manipulation seat 162, and its axial direction is preferably perpendicular to the functional manipulation seat 162. The bevel spur gear 174 has a gear rotation shaft 1743, a bevel gear portion 1741 and a spur gear portion 1742. Wherein the gear rotation shaft 1743 is rotatably disposed to the functional manipulation seat 162 along the vertical direction. The spur gear portion 1742 is horizontally fixed to the bottom end of the gear rotation shaft 1743 and is engaged with the transfer gear 1664. The bevel gear portion 1741 is disposed on the upper side of the spur gear portion 1742 around the gear rotation shaft 1743 and is engaged with the bevel gear 173. Thus, the power from the second driving device 144 transferred through the second transmission assembly 95 may be transferred to the functional gear 172.

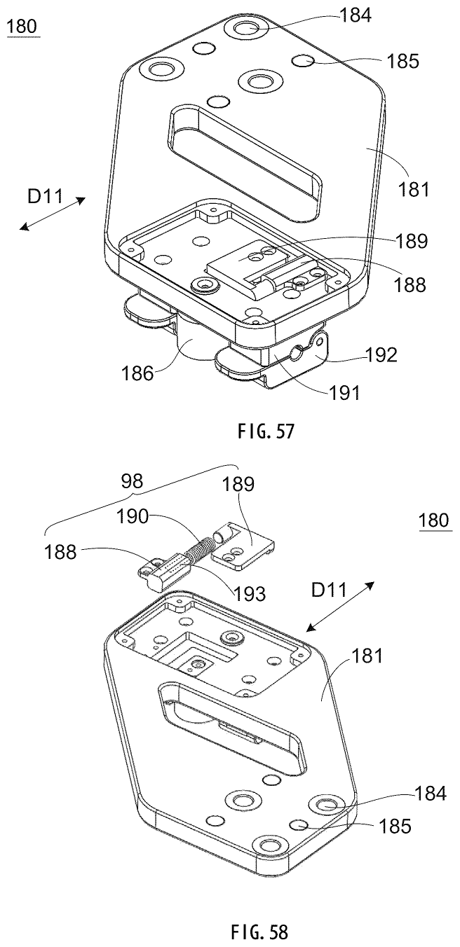

Please refer to FIGS. 35, 36 and 56 to 58 below, the slave-side execution apparatus 100 further includes a second carrying device 180 disposed on the main body power device 110 and located in front of the first carrying device 160 along the conveying direction DF. For example, the end of the machine frame 111 of the main body power device 110 is provided with a mounting portion 129 (see FIG. 39), which may extend out of the housing 112. The second carrying device 180 includes a supporting seat 181 detachably mounted on the mounting portion 129. In a preferred embodiment, a positioning portion 184 is provided on the supporting seat 181, and a positioning mating portion 130 is provided on the mounting portion 129 of the main body power device 110. The positioning portion 184 is connectable to the positioning mating portion 130, e.g., a column-hole mating. In this embodiment, a magnetic member 185 is also provided on the supporting seat 181, a magnetic attraction member 131 is provided on the mounting portion 129, and they may be connected in docking. For example, after the positioning is completed using the positioning portion 184 and the positioning mating portion 130, a stable connection is formed under the corresponding attraction effect between the magnetic member 185 and the magnetic attraction member 131.

The second carrying device 180 further has a guide assembly 96 for guiding the guidewire 12 and/or the catheter 11. The guide assembly 96 is disposed in front of the first manipulation device 93 and the second manipulation device 90 along the conveying direction DF. Specifically, the guide assembly 96 includes a catheter guiding device 182 and a catheter supporting device 183, both of which are provided on the supporting seat 181. The catheter guiding device 182 is used to clamp or release the guidewire 12 and/or the catheter 11, and the catheter supporting device 183 is configured to allow the guidewire 12 and/or the catheter 11 to pass through so as to support the guidewire 12 and/or the catheter 11.

Wherein, the catheter guiding device 182 includes a first guide roller 186, a second guide roller 187, and a second biasing assembly 98.

The first guide roller 186 and the second guide roller 187 are disposed opposite to each other, and the axes of the two are substantially parallel to each other and the two are rotatable about their own axes relative to the supporting seat 181.

The second biasing assembly 98 includes a fixed block 188, a pushing block 189 and a third elastic member 190. The fixed block 188 is disposed on the bottom side of the supporting seat 181, and a guide groove 193 communicating with the outside is provided in the fixed block 188. The guide groove 193 extends along the first additional direction D11, wherein the first additional direction D11 is perpendicular to the plane determined by the conveying direction DF and the axis of the second guide roller 187. The pushing block 189 at least partially extends into the guide groove 193. The third elastic member 190 is located in the guide groove 193 and connected between the fixed block 188 and the pushing block 189. The third elastic member extends along the first additional direction D11 for providing a force to the pushing block 189 that causes the pushing block 189 to tend to move in a direction close to the fixed block 188, i.e., causes the pushing block 189 to move towards the fixed block 188 along the first additional direction D11.

The first guide roller 186 is fixedly disposed on the upper side of the supporting seat 181. The second guide roller 187 is located on the upper side of the supporting seat 181 and is connected to the pushing block 189 so that the second guide roller 187 is movable along a direction close to or away from the first guide roller 186. The elastic force of the third elastic member 190 causes the second guide roller 187 to tend to move closer to the first guide roller 186 so as to clamp the guidewire 12 and/or the catheter 11. That is, under the action of the second biasing assembly 98, the second guide roller 187 is movable relative to the first guide roller 186 along the first additional direction D11 so as to be able to release or clamp the guidewire 12 and/or the catheter 11 with the first guide roller 186. Therefore, when the pushing block 189 is manipulated by the operator, the second guide roller 187 may be moved close to or away from the first guide roller 186, thereby clamping or releasing the guidewire 12 and/or the catheter 11.

The catheter supporting device 183 includes a catheter supporting portion 191 and a catheter cover portion 192. The catheter supporting portion 191 is connected to the supporting seat 181. A semi-catheter groove is provided on the catheter supporting portion 191. The catheter cover portion 192 may be connected to the catheter supporting portion 191 in an openable and closable manner, and a semi-catheter groove is also provided on the catheter cover portion 192. When the catheter cover portion 192 covers the catheter supporting portion 191, the two semi-catheter grooves form a complete catheter groove to support the guidewire 12 and/or the catheter 11 and allow the guidewire 12 and/or the catheter 11 to pass through. Preferably, a snapping structure may be provided on the catheter supporting portion 191 and the catheter cover portion 192 to form snap-fit connection when the catheter cover portion 192 covers the catheter supporting portion 191.

In addition, in this embodiment, the first carrying device 160 and/or the second carrying device 180 may be configured as passive consumable components. For example, the first carrying device 160 and the second carrying device 180 may both be configured as passive consumables made of plastic. Preferably, an isolation component such as a sterile bag or a sterile curtain may be provided between the first carrying device 160 and the movable power device 140, and between the second carrying device 180 and the main body power device 110. In this way, each patient may correspond to a set of disposable passive consumables, and thus the workload of sterilization and disinfection is reduced. Moreover, the main body power device 110 and the movable power device 140 may be reused, thereby reducing the use cost of the hospital.

During the use of the slave-side execution apparatus 100 of the present application, it is preferably obliquely disposed, wherein the end (front end) of the slave-side execution apparatus 100 along the first conveying direction DF1 is lower than the end (rear end) of the slave-side execution apparatus 100 along the second conveying direction DF2. In other words, the slave-side execution apparatus 100 is disposed obliquely in the direction toward the patient, and the end of the slave-side execution apparatus 100 along the first conveying direction DF1 faces the patient. Wherein the oblique angle of the slave-side execution apparatus 100 is θ. That is, the conveying direction DF and the horizontal plane have an included angle of θ.

Therefore, the total gravity of the movable seat 120, the movable power device 140 and the first carrying device 160 has a component force F along the oblique direction of the slave-side execution apparatus 100, and F=mg sin θ, wherein m is the total mass of the movable seat 120, the movable power device 140 and the first carrying device 160, and g is the gravitational acceleration.

The processor module is signal-connected to the first force sensor 122 and the second force sensor 123, and is configured to calculate a resistance F_{R} experienced by the catheter 11 based on the component force F and the pressure sensed by the first force sensor 122 and/or the second force sensor 123.

Exemplarily, the pressure sensed by the first force sensor 122 is F₁, and the pressure sensed by the second force sensor 123 is F₂. When the movable power device 140 is stationary, the first force sensor 122 has a reading due to the obliquity of the slave-side execution apparatus 100, and at this time F₁=F.

When the catheter 11 is resisted during a uniform advancement, if the first force sensor 122 has a reading, F_{R}=F−F1. If the second force sensor 123 has a reading, F_{R}=F+F2.

When the catheter 11 is resisted during an acceleration advancement or a deceleration advancement at the acceleration a, the movable seat 120 has inertia, and the inertial force Fᵢ=ma. In this case, if it is during the acceleration advancement and the first force sensor 122 has a reading, F_{R}=F−Fᵢ−F₁. If it is during the acceleration advancement and the second force sensor 123 has a reading, F_{R}=F−Fᵢ+F₂. If it is during the deceleration advancement and the first force sensor 122 has a reading, F_{R}=F+Fᵢ−F₁. If it is during the deceleration advancement and the second force sensor 123 has a reading, F_{R}=F+Fᵢ+F₂.

As a result, the resistance experienced by the catheter 11 when advancing and retracting and its changes may be accurately obtained, which is conducive to achieving more accurate control of the advancing and retracting speeds and improving safety.

Reference is made to the description of the first embodiment for the parts which are not introduced in the second embodiment.

The processes and steps described in all the above preferred embodiments are only examples. Unless adverse effects occur, various processing operations may be performed in a sequence different from the sequence of the above processes. The sequence of steps in the above processes may also be added, merged or deleted according to actual needs.

In understanding the scope of the present application, the term “including” and its derivatives as used herein are intended to be open terms, which specify the presence of stated features, elements, components, groups, wholes and/or steps, but do not exclude the presence of other unstated features, elements, components, groups, wholes and/or steps. This concept also applies to words with similar meanings such as the terms “comprising”, “having” and their derivatives.

The term “attached” or “attaching” as used herein includes: a configuration in which an element is directly fixed to another element by fixing the element directly to another element; a configuration in which the element is indirectly fixed to another element by fixing the element to the intermediate element(s) which in turn are fixed to another element; and a configuration in which one element is integrated with another element, that is, one element is essentially a part of another element. This definition also applies to words having similar meanings such as “connected”, “joined”, “coupled”, “mounted”, “bonded”, and “fixed” and their derivatives. Finally, terms of degree such as “substantially”, “about” and “approximately” as used herein mean an amount of deviation of the modified term such that the end result is not significantly changed.

Unless otherwise defined, the technical and scientific terms used herein have the same meanings as commonly understood by those skilled in the technical field of the present application. The terms used herein are only for describing specific implementation purposes, and are not intended to limit the present application. Feature(s) described in one embodiment herein may be applied to another embodiment alone or in combination with other features, unless the feature(s) are not applicable in the other embodiment or otherwise stated.

The present application has been described through the above-mentioned embodiments, but it should be understood that the above-mentioned embodiments are only for the purpose of illustrations and descriptions, and are not intended to limit the present application to the scope of the described embodiments. Moreover, those skilled in the art may understand that the present application is not limited to the above-mentioned embodiments. Various changes and modifications may be made based on the enlightenment of the present application, and these changes and modifications fall within the protection scope claimed by the present application.

What is claimed is:

1. A slave-side execution apparatus for conveying a guidewire and/or a catheter, comprising:
a main body power device used to be installed beside a surgical bed and being immovable relative to the surgical bed, the main body power device comprising a main body driving device for providing a driving force;
a movable power device connected to the main power device and used to move forward and backward relative to the main power device along a conveying direction of the guidewire and/or the catheter under the driving of the main body driving device, the movable power device comprising a first driving device for providing a driving force;
a first carrying device detachably connected to the movable power device and being movable synchronously with the movable power device along the conveying direction relative to the main body power device, the first carrying device being used to carry a medical apparatus having the catheter; and
a first manipulation device disposed on the first carrying device and used to control the guidewire to move forward and backward relative to the first carrying device along the conveying direction under the driving of the first driving device.

2. The slave-side execution apparatus of claim 1, wherein,
the movable power device comprises a second driving device for providing a driving force; and
the first carrying device comprises a second manipulation device used to contact the medical apparatus, and the second manipulation device being connected to the second driving device to manipulate the medical apparatus to perform a medical function under the driving of the second driving device.

3. The slave-side execution apparatus of claim 2, wherein the slave-side execution apparatus further comprises:
a movable assembly disposed on the main body power device and configured to be movable forward and backward relative to the main body power device along the conveying direction under the driving of the main body driving device, wherein the movable power device is connected to the movable assembly;
a first transmission device connected between the first driving device and the first manipulation device for transferring the driving force of the first driving device to the first manipulation device; and
a second transmission device connected between the second driving device and the second manipulation device for transferring the driving force of the second driving device to the second manipulation device.

4. The slave-side execution apparatus of claim 3, wherein,
the first manipulation device comprises at least one pair of rollers which are arranged oppositely, the axes of the rollers are parallel to each other and the rollers are configured to be rotatable about their own axes, and the pair of the rollers are used to jointly clamp the guidewire; and
the first transmission device is connected between the first driving device and at least one of the pair of the rollers to rotate the at least one of the pair of the rollers.

5. The slave-side execution apparatus of claim 3, wherein the first transmission device comprises:
a first power shaft disposed on the movable power device and configured to be rotatable about its own axis relative to the movable power device;
a first transmission assembly disposed on the movable power device and connected between the first driving device and the first power shaft such that the first power shaft is rotatable under the driving of the first driving device; and
a first power transfer device disposed on the first carrying device and connected between the first manipulation device and the first power shaft such that the first manipulation device is driven to operate by the first power shaft drives.

6. The slave-side execution apparatus of claim 5, wherein the first manipulation device comprises:
a first manipulation device base connected to the first carrying device;
at least one first roller disposed on the first manipulation device base, an axis of the first roller being not parallel to the conveying direction, the first roller being configured to be rotatable about its axis relative to the first manipulation device base; and
at least one second roller disposed on the first manipulation device base and arranged opposite to the first roller so as to clamp the guidewire along with the first roller, the second roller being configured to be rotatable about its axis relative to the first manipulation device base;
wherein the first roller and the second roller are both used to be connected to the first transmission device so as to rotate under the driving of the first transmission device, and the slave-side execution apparatus is configured such that an rotation direction of the first roller is opposite to an rotation direction of the second roller.

7. The slave-side execution apparatus of claim 6, wherein the first manipulation device further comprises:
a second fixed seat disposed on the first manipulation device base, wherein the second roller is disposed on the second fixed seat; and
a first movable seat disposed on the first manipulation device base and being movable between a first position and a second position relative to the second fixed seat along a first direction, wherein the first roller is disposed on the first movable seat;
wherein the first direction is perpendicular to a plane determined by the conveying direction and the axis of the first roller, and a distance between the first movable seat and the second fixed seat when the first movable seat is in the first position is less than a distance between the first movable seat and the second fixed seat when the first movable seat is in the second position.

8. The slave-side execution apparatus of claim 7, wherein the first manipulation device further comprises:
a first biasing assembly connected between the second fixed seat and the first movable seat for providing a force to move the first movable seat away from the second fixed seat along the first direction; and
a first pressing device disposed on the first manipulation device base and connected to the first movable seat for operation by a user to bring the first movable seat close to the second fixed seat along the first direction.

9. The slave-side execution apparatus of claim 8, wherein the first pressing device comprises:
a cam connected to the first manipulation device base and being rotatable relative to the first manipulation device base about a rotation axis extending along a second direction, wherein the second direction is perpendicular to the first direction and parallel to a plane determined by the conveying direction and the axis of the first roller, and a circumferential surface of the cam is connected to a side of the first movable seat facing away from the second fixed seat; and
a wrench connected to the cam for operation by the user to rotate the cam relative to the first manipulation device base.

10. The slave-side execution apparatus of claim 7, wherein,
the first power transfer device comprises a first transmission shaft used to be connected to the first driving device so as to rotate under the driving of the first driving device; and
the first manipulation device further comprises:
a first roller transmission device used to connect the first transmission shaft and the first roller so that the rotation direction of the first roller is the same as a rotation direction of the first transmission shaft, and
a second roller transmission device used to connect the first transmission shaft and the second roller so that the rotation direction of the second roller is opposite to the rotation direction of the first transmission shaft.

11. The slave-side execution apparatus of claim 10, wherein the first roller transmission device comprises at least one first manipulation synchronous belt, the first transmission shaft and the first roller are both connected with a structure for connecting the first manipulation synchronous belt, and the slave-side execution apparatus is configured such that the first transmission shaft drives all the first rollers to rotate via the first manipulation synchronous belt.

12. The slave-side execution apparatus of claim 11, wherein the first manipulation device comprises N first rollers and N first manipulation synchronous belts, where N is an integer greater than or equal to 1, wherein,
each of the first rollers is connected to the first transmission shaft via one first manipulation synchronous belt; or
each of one part of the N first rollers is connected to the first transmission shaft via one first manipulation synchronous belt, and each of the other part of the N first rollers is connected to another first roller via one first manipulation synchronous belt.

13. The slave-side execution apparatus of claim 11, wherein the first manipulation device comprises N first rollers and N first manipulation synchronous belts, where N is an integer greater than or equal to 1, wherein,
the first one of the N first rollers is connected to the first transmission shaft via one first manipulation synchronous belt, and each of the other first rollers is connected to the previous first roller via one first manipulation synchronous belt.

14. The slave-side execution apparatus of claim 13, wherein the first manipulation device further comprises:
a first additional roller disposed on the first movable seat, the first additional roller being configured to be rotatable about its axis relative to the first movable seat;
a second additional roller disposed on the second fixed seat and arranged opposite to the first additional roller so as to clamp the guidewire along with the first additional roller, the second additional roller being configured to be rotatable about its axis relative to the second fixed seat;
a first additional roller transmission device used to connect the first additional roller and the Nth one of the first rollers so that the first additional roller is rotated in the same direction as a rotation direction of the first transmission shaft under the driving of the Nth one of the first rollers; and
an encoder connected to the second additional roller and used to detect a rotation angle of the second additional roller.

15. The slave-side execution apparatus of claim 14, wherein,
the second additional roller has a second additional roller axle;
the movable power device also comprises a third rotating shaft disposed on the movable power device, the third rotating shaft being used to be connected to the second additional roller axle and rotate synchronously with the second additional roller axle, wherein the encoder is disposed on the third rotating shaft.

16. The slave-side execution apparatus of claim 10, wherein the second roller transmission device comprises:
a fourth transmission shaft disposed on the first manipulation device base;
a first transmission device for the second roller used to connect the first transmission shaft and the fourth transmission shaft so that the fourth transmission shaft is rotated under the driving of the first transmission shaft; and
a second transmission device for the second roller used to connect the fourth transmission shaft and the second roller so that the second roller is rotated under the driving of the fourth transmission shaft.

17. The slave-side execution apparatus of claim 16, wherein one of the first transmission device for the second roller and the second transmission device for the second roller is configured to be driven by a synchronous belt, and the other of the first transmission device for the second roller and the second transmission device for the second roller is configured to be driven by a gear.

18. The slave-side execution apparatus of claim 10, wherein a component of the first roller transmission device for connecting the first transmission shaft and a component of the second roller transmission device for connecting the first transmission shaft are both synchronous belts.

19. The slave-side execution apparatus of claim 5, wherein the first manipulation device comprises:
a driving roller member disposed on the first carrying device, the driving roller member comprising a driving roller and a driving gear coaxially connected, wherein the driving gear is in transmission connection with the first power transfer device so as to rotate about its own axis relative to the first carrying device under the action of the first power transfer device; and
a driven roller member disposed on the first carrying device, the driven roller member comprising a driven roller arranged opposite to the driving roller, the driven roller being configured to be rotatable about its own axis relative to the first carrying device and movable relative to the driving roller along a first direction, so as to be able to clamp or release the guidewire with the driving roller, wherein the first direction is perpendicular to a plane determined by the conveying direction and an axis of the driven roller.

20. The slave-side execution apparatus of claim 19, wherein the driven roller member further comprises:
a fixed frame disposed on the first carrying device, the fixed frame having a third guide rail extending along the first direction;
an extrusion frame disposed on the third guide rail and being movable along the third guide rail, the driven roller being disposed on the extrusion frame; and
a first elastic member connected between the fixed frame and the extrusion frame, the first elastic member extending along the first direction for providing a force that brings the extrusion frame close to the driving roller along the first direction.

21. The slave-side execution apparatus of claim 20, wherein the first manipulation device further comprises a manipulation rod assembly comprising:
a manipulation portion used for being operated by a user and exposed from the first carrying device;
a pivoting portion connected to the first carrying device and configured to be rotatable about its own axis relative to the first carrying device;
an extension portion connected between the pivoting portion and the manipulation portion so that the manipulation portion is capable of rotating synchronously with the pivoting portion about an axis of the pivoting portion; and
a pushing portion provided to the extension portion for contacting the extrusion frame to enable the pushing portion to push the extrusion frame away from the driving roller along the first direction when the pivoting portion is rotated.

22. The slave-side execution apparatus of claim 5, wherein a position of the first power transfer device corresponds to a position of the first power shaft, and the first power transfer device is engaged with the first power shaft, the first power transfer device comprises a transfer synchronous roller, and a synchronous belt transmission is formed between the first power transfer device and the first manipulation device.

23. The slave-side execution apparatus of claim 5, wherein the second transmission device comprises:
a second power shaft disposed on the movable power device and configured to be rotatable about its own axis relative to the movable power device;
a second transmission assembly disposed on the movable power device and connected between the second driving device and the second power shaft so that the second power shaft is rotated under the driving of the second driving device; and
a second power transfer device disposed on the first carrying device and connected between the second manipulation device and the second power shaft so that the second power shaft drives the second manipulation device to operate.

24. The slave-side execution apparatus of claim 23, wherein,
the first power shaft is arranged to penetrate through the movable power device, and an end of the first power shaft facing the first carrying device is in transmission connection with the first manipulation device;
the first transmission assembly is connected between the first driving device and an end of the first power shaft facing away from the first carrying device;
the second power shaft is arranged to penetrate through the movable power device, and an end of the second power shaft facing the first carrying device is in transmission connection with the second manipulation device; and
the second transmission assembly is connected between the second driving device and an end of the second power shaft facing away from the first carrying device.

25. The slave-side execution apparatus of claim 23, wherein,
the first carrying device comprises a functional manipulation seat for mounting the medical apparatus, the functional manipulation seat comprising a first side of the functional manipulation seat and a second side of the functional manipulation seat which are arranged opposite to each other, wherein the medical apparatus is detachably connected to the first side of the functional manipulation seat, and the functional manipulation seat is provided with an operation opening; and
the second manipulation device comprises:
a functional gear rotatably disposed at the second side of the functional manipulation seat and at least partially exposed from the operation opening to the first side of the functional manipulation seat, so as to be able to form an engagement with the medical apparatus, and
a bevel gear coaxially connected with the functional gear and used to be connected to the second power transfer device.

26. The slave-side execution apparatus of claim 25, wherein the first carrying device further comprises:
a first bracket disposed on the first side of the functional manipulation seat;
a second bracket disposed on the first side of the functional manipulation seat and spaced apart from the first bracket along the conveying direction so as to support the medical apparatus along with the first bracket, the second bracket being configured to be movable relative to the functional manipulation seat along the conveying direction; and
a second elastic member connected to the second bracket for applying a force to the second bracket to move the second bracket away from the first bracket along the conveying direction.

27. The slave-side execution apparatus of claim 23, wherein a position of the second power transfer device corresponds to a position of the second power shaft, and the second power transfer device is engaged with the second power shaft, the second power transfer device comprises a transfer gear, and a gear engaging transmission is formed between the second power transfer device and the second manipulation device.

28. The slave-side execution apparatus of claim 3, wherein the main body power device further comprises:
a machine frame on which the main body driving device and the movable assembly are both provided; and
a first guide rail disposed on the machine frame and extending along the conveying direction;
wherein the movable assembly is connected to the first guide rail and is configured to move along the first guide rail under the driving of the main body driving device.

29. The slave-side execution apparatus of claim 3, wherein the movable assembly comprises:
a movable frame configured to be moved along the first guide rail under the driving of the main body driving device, the movable frame having a second guide rail extending along the conveying direction;
a movable seat disposed on the second guide rail and being movable along the second guide rail, the movable power device being connected to the movable seat; and
a force sensor disposed between the movable seat and the movable frame to sense a force experienced by the movable seat when the catheter is conveyed.

30. The slave-side execution apparatus of claim 1, wherein,
the slave-side execution apparatus further comprises a guide assembly disposed in front of the first manipulation device along the conveying direction; and
the guide assembly comprises at least one pair of rollers arranged opposite to each other, the axes of the rollers are parallel to each other and the rollers are configured to be rotatable about their own axes, and one pair of rollers is used to jointly clamp the guidewire and/or the catheter.

31. The slave-side execution apparatus of claim 1, wherein the slave-side execution apparatus also comprises a second carrying device disposed on the main body power device and located in front of the first carrying device along the conveying direction, and the second carrying device has a guide assembly for guiding the guidewire and/or the catheter.

32. The slave-side execution apparatus of claim 31, wherein,
the second carrying device further comprises a supporting seat connected to the movable power device;
the guide assembly comprises:
a catheter guiding device disposed on the supporting seat so as to be able to clamp or release the guidewire and/or the catheter, and
a catheter supporting device disposed on the supporting seat and configured to allow the guidewire and/or the catheter to pass through to support the guidewire and/or the catheter.

33. The slave-side execution apparatus of claim 32, wherein the catheter guiding device comprises:
a first guide roller disposed on the supporting seat and configured to be rotatable about its own axis relative to the supporting seat; and
a second guide roller disposed on the supporting seat and arranged opposite to the first guide roller, the second guide roller being configured to be rotatable about its own axis relative to the supporting seat and configured to be movable relative to the first guide roller along a first additional direction so as to be able to release or clamp the guidewire and/or the catheter with the first guide roller, wherein the first additional direction is perpendicular to a plane determined by the conveying direction and an axis of the second guide roller.

34. The slave-side execution apparatus of claim 33, wherein the second carrying device further comprises:
a fixed block disposed on the supporting seat and having a guide groove extending along the first additional direction;
a pushing block at least partially extending into the guide groove, the second guide roller being provided on the pushing block; and
a third elastic member disposed in the guide groove and connected between the fixed block and the pushing block, the third elastic member extending along the first additional direction to apply a force to the pushing block that causes the pushing block to move close to the first guide roller along the first additional direction.

35. The slave-side execution apparatus of claim 1, further comprising:
a second power assembly used to be installed beside a surgical bed and being immovable relative to the surgical bed, the second power assembly comprising a fourth driving device for providing a driving force; and
a second conveying assembly detachably connected to the second power assembly for conveying the guidewire and/or the catheter under the driving of the fourth driving device;
wherein the second power assembly is used to be installed in front of the main body power device along the conveying direction of the guidewire and/or the catheter.

36. The slave-side execution apparatus of claim 35, wherein the second conveying assembly comprises:
a second conveying assembly base detachably connected to the second power assembly;
at least one third roller disposed on the second conveying assembly base, an axis of the third roller being not parallel to the conveying direction; and
at least one fourth roller disposed on the second conveying assembly base and arranged opposite to the third roller, so as to clamp the guidewire and/or the catheter along with the third roller;
wherein the third roller is used for rotating about its own axis under the driving of the fourth driving device.

37. The slave-side execution apparatus of claim 36, wherein the second conveying assembly comprises:
a third roller assembly disposed on the second conveying assembly base and comprising at least one the third roller;
a fourth roller assembly comprises:
a fifth movable seat disposed on the second conveying assembly base and being movable relative to the second conveying assembly base along a first additional direction, wherein the first additional direction is perpendicular to the axis of the third roller, and
at least one the fourth roller disposed on the fifth movable seat, the fourth roller being arranged opposite to the corresponding third roller along the first additional direction; and
a position adjustment assembly connected to the fifth movable seat for driving the fifth movable seat to move along the first additional direction under the driving of a third driving device, so as to change a distance the fourth roller and the third roller.

38. The slave-side execution apparatus of claim 37, wherein the position adjustment assembly comprises:
an adjusting gear disposed on the second conveying assembly base and used for rotating relative to the second conveying assembly base under the driving of the third driving device; and
an adjusting rack engaged with the adjusting gear, the adjusting rack extending along the first additional direction and connected to the fifth movable seat.

39. The slave-side execution apparatus of claim 37, wherein the third roller assembly comprises a plurality of the third rollers,
the third roller assembly is configured such that a part of the third rollers are used for rotating about their own axes under the driving of the fourth driving device and the other third rollers are rotated about their own axes under the driving of another third roller.

40. The slave-side execution apparatus of claim 39, wherein the second conveying assembly further comprises a roller transmission assembly, a first end of the roller transmission assembly is connected to the third roller and a second end of the roller transmission assembly is connected to the fourth roller so that the third roller drives the fourth roller to rotate in an opposite direction at the same rotational speed, and
wherein the roller transmission assembly is configured such that the second end of the roller transmission assembly is movable relative to the first end of the roller transmission assembly along the first additional direction, so as to adapt to the movement of the fifth movable seat along the first additional direction.

41. The slave-side execution apparatus of claim 40, wherein the roller transmission assembly comprises N transmission gears that are engaged sequentially, N being an even number greater than or equal to 4,
wherein the first one of the transmission gears is disposed at the first end of the roller transmission assembly and is rotated synchronously with the third roller, the last one of the transmission gears is disposed at the second end of the roller transmission assembly and is rotated synchronously with the fourth roller, and the last one of the transmission gears is movable relative to the first one of the transmission gears along the first additional direction.

42. The slave-side execution apparatus of claim 41, wherein the third roller is configured as the same roller as the fourth roller, and the roller transmission assembly has a transmission ratio of 1:1.

43. The slave-side execution apparatus of claim 41, wherein the roller transmission assembly further comprises:
a first connecting arm comprising a first end of the first connecting arm and a second end of the first connecting arm arranged oppositely, the first one of the transmission gears being disposed on the first end of the first connecting arm; and
a second connecting arm comprising a first end of the second connecting arm and a second end of the second connecting arm arranged oppositely, the last one of the transmission gears being disposed on the second end of the second connecting arm,
wherein the first end of the second connecting arm is rotatably connected to the second end of the first connecting arm about a third axis, the third axis is parallel to an axis of the third roller, and N transmission gears are disposed on the first connecting arm and the second connecting arm.

44. The slave-side execution apparatus of claim 43, wherein the third axis coincides with an axis of one transmission gear other than the first one of the transmission gears and the last one of the transmission gears.

45. The slave-side execution apparatus of claim 40, wherein the fourth roller assembly comprises a plurality of the fourth rollers, and
the fourth roller assembly is configured such that a part of the fourth rollers are used for rotating about their own axes under the driving of the third roller and the other fourth rollers are rotated about their own axes under the driving of another fourth roller.

46. The slave-side execution apparatus of claim 40, wherein the third roller assembly comprises a plurality of the third rollers, and the fourth roller assembly comprises a plurality of the fourth rollers,
wherein the Pth one of the third rollers is used for rotating about its own axis under the driving of the fourth driving device, the first end of the roller transmission assembly is connected to the Qth one of the third rollers, the second end of the roller transmission assembly is connected to the Qth one of the fourth rollers, and P is not equal to Q.

47. The slave-side execution apparatus of claim 37, wherein the second conveying assembly further comprises a biasing member for applying a force to the fifth movable seat to move away from the third roller along the first additional direction.

48. A medical system, comprising:
a first supporting frame used to be disposed beside a surgical bed;
the slave-side execution apparatus of claim 1, wherein the main body power device is connected to the first supporting frame; and
a medical apparatus detachably connected to the first carrying device.

49. A medical system, comprising:
a first supporting frame used to be disposed beside a surgical bed;
a second supporting frame used to be disposed beside the surgical bed and located in front of the first supporting frame along the conveying direction of a guidewire and/or a catheter;
the slave-side execution apparatus of claim 35, wherein the main body power device is connected to the first supporting frame, and the second power assembly is connected to the second supporting frame; and
a medical apparatus detachably connected to the first carrying device.

50. The medical system of claim 48, further comprising:
a master-side operating apparatus; and
a processor module signal-connected to both the master-side operating apparatus and the slave-side execution apparatus.

51. The medical system of claim 49, further comprising:
a master-side operating apparatus; and
a processor module signal-connected to both the master-side operating apparatus and the slave-side execution apparatus.
